(19) United States
(12) Patent Application Publication
(10) Pub. No.: US 2023/0165678 A1
(43) Pub. Date: Jun. 1, 2023

(54) DEVICES AND METHODS FOR ADJUSTING THE TENSIONS OF LEAFLETS MOUNTED WITHIN PROSTHETIC VALVES

(71) Applicant: Edwards Lifesciences Corporation, Irvine, CA (US)

(72) Inventors: Noam Nir, Pardes-Hanna (IL); Michael Bukin, Pardes Hanna (IL)

(21) Appl. No.: 18/096,728

(22) Filed: Jan. 13, 2023

Related U.S. Application Data
(63) Continuation of application No. PCT/US2021/041720, filed on Jul. 15, 2021.
(60) Provisional application No. 63/052,107, filed on Jul. 15, 2020.

(51) Int. Cl. A61F 2/24

(52) U.S. Cl. CPC A61F 2/2418; A61F 2220/0025; A61F 2250/0012

(57) ABSTRACT

The present invention relates to implantable prosthetic devices, such as prosthetic heart valves with mechanisms for adjusting the tension of leaflets mounted therein, and to methods, adjustment assemblies and delivery assemblies for, and including, such devices.

### CROSS REFERENCE TO RELATED APPLICATIONS

This application is a continuation of International Application No. PCT/US2021/041720, filed Jul. 15, 2021, which claims benefit of U.S. Provisional Application No. 63/052,107, filed on Jul. 15, 2020, the contents of each of which are herein incorporated by reference in their entirety.

### FIELD OF THE INVENTION

The present invention relates to implantable prosthetic devices, such as prosthetic heart valves with mechanisms for adjusting the tension of leaflets mounted therein, and to methods, adjustment assemblies and delivery assemblies for, and including, such devices.

### BACKGROUND OF THE INVENTION

Native heart valves, such as the aortic, pulmonary and mitral valves, function to assure adequate directional flow from, and to, the heart, and between the heart’s chambers, to supply blood to the whole cardiovascular system. Various valvular diseases can render the valves ineffective and require replacement with artificial valves. Surgical procedures can be performed to repair or replace a heart valve. Since surgeries are prone to an abundance of clinical complications, alternative less invasive techniques of delivering a prosthetic heart valve over a catheter and implanting it over the native malfunctioning valve have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient’s femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

An implantation procedure of a prosthetic valve includes expansion thereof at the implantation site, to an expansion diameter that matches the patient’s anatomy. Different patient may have various anatomical structures, that will require the prosthetic valve to expand to the appropriate diameter matching the specific patient’s anatomy and/or pathology at the site of implantation. It may be desirable to provide prosthetic valves that can be expanded to a wide range of final expansion diameters, in order to lower production costs and simplify inventory management. However, one of the limiting factors for the diameters to which a prosthetic valve can be expanded relates to the leaflets mounted within the frame of the prosthetic valve, which need to properly coapt so as to prevent retrograde blood from flowing between leaflets during diastole. Overexpansion or under-expansion of the frame may result in overstretching or loosening of the leaflets, which in turn may result in impaired coaptation. Thus, a need exists for mechanisms that will allow prosthetic valve to be utilized in a wide range of expansion dimeters, while preserving adequate coaptation between the leaflets during diastole.

### SUMMARY OF THE INVENTION

The present disclosure is directed toward prosthetic heart valves that include an adjustable commissure supports to which leaflet assemblies are attached, wherein the adjustable commissure supports include adjustable arms that can be either rotated or twisted, thereby enabling the tension of the leaflets attached thereto to be adjusted, so as to match the desired expansion diameter of the frame, and allow proper coaptation between the leaflets when the frame is expanded to the desired diameter.

According to one aspect of the invention, there is provided a prosthetic valve comprising a frame movable between a radially compressed configuration and a radially expanded configuration, a leaflet assembly mounted within the frame, and a plurality of adjustable commissure supports.

The leaflet assembly comprises a plurality of leaflets configured to regulate flow through the prosthetic valve. Each leaflet comprises a rounded cusp end portion defining a cusp edge, a free edge opposite to the cusp edge, and a pair of oppositely-directed tabs separating the cusp edge and the free edge.

Each adjustable commissure support comprises a support base attached to the frame, and at least one adjustable arm extending proximally from the support base. Each adjustable arm comprises an engagement portion.

The tabs are attached to the adjustable arms. The adjustable arms are configured to rotate or twist when rotational force is applied to the engagement portions. Rotation or twisting of the adjustable arms causes the tabs attached thereto, to wind around the adjustable arms.

According to some embodiments, the support base is integrally formed with the frame.

According to some embodiments, the tabs are wrapped over the adjustable arms.

According to some embodiments, each adjustable arm has a non-circular cross section.

According to some embodiments, each adjustable arm comprises a plurality of apertures.

According to some embodiments, the apertures are axially spaced apart from each other.

According to some embodiments, the tabs are sutured to the adjustable arms via sutures through the tabs and the apertures.

According to some embodiments, the sutures extend through portions of the tab disposed over opposite sides of the adjustment arms.

According to some embodiments, the engagement portion comprises a non-cylindrical socket.

According to some embodiments, the engagement portion comprises a proximally oriented extension formed as a drive-screw coupler.

According to some embodiments, the engagement portion comprises an angled surface.

According to some embodiments, the adjustable arms are rigidly attached to the support base.

According to some embodiments, the adjustable arms are integrally formed with the support base.

According to some embodiments, the adjustable arms are configured to twist between the support base and the engagement portions, upon application of rotational force to the engagement portions.

According to some embodiments, the adjustable arms comprise a plastically deformable material.

According to some embodiments, the adjustable arms are rotationally coupled to the support base.

According to some embodiments, the adjustable arms are threadedly coupled to the support base.

According to some embodiments, the support base comprises at least one base threaded portion, wherein each adjustable arm comprises an adjustable arm threaded portion, configured to threadedly engage with the base threaded portion.

According to some embodiments, the support base further comprises at least one offsetting extension, extending radially therefrom, wherein the at least one adjustable arm extends from the respective offsetting extension.

According to some embodiments, the at least one offsetting extension extends radially away from the support base.

According to some embodiments, the at least one offsetting extension extends radially inward with respect to the support base.

According to some embodiments, the offsetting extension is integrally formed with the support base.

According to some embodiments, each adjustable commissure support further comprises a commissure support fastening extension.

According to some embodiments, the adjustable commissure supports are attached to the frame via the commissure support fastening extensions, such that the adjustable arms are positioned radially inward with respect to the frame.

According to some embodiments, each adjustable commissure support comprises a single adjustable arm.

According to some embodiments, two tabs of adjacent leaflets of the leaflet assembly are attached to the adjustable commissure support, such that both tabs are wrapped over the single adjustable arm.

According to some embodiments, the leaflets are asymmetric leaflets, each asymmetric leaflet having a short tab and an opposite long tab, wherein the tabs of adjacent leaflets are wrapped over the single adjustable arm such that the short tab of one leaflet is wrapped around the adjustable arm, in direct contact therewith, and the long tab of the adjacent leaflet is wrapped over the short tab.

According to some embodiments, the length of the long tab is at least 10% as great as than the length of the short tab.

According to some embodiments, the length of the long tab is at least 20% as great as than the length of the short tab.

According to some embodiments, the length of the long tab is at least 50% longer than the length of the short tab.

According to some embodiments, the length of the long tab is at least 100% longer than the length of the short tab.

According to some embodiments, each adjustable commissure support comprises two adjustable arms that are laterally spaced from each other and define a gap therebetween.

According to some embodiments, two tabs of adjacent leaflets of the leaflet assembly are attached to the adjustable commissure support, such that each tab is wrapped over one of the adjustable arms.

According to some embodiments, both tabs are wrapped over the respective adjustable arms in opposite directions with respect to each other.

According to some embodiments, the gap is at least two times larger than the thickness of the tabs.

According to some embodiments, the gap is at least four times larger than the thickness of the tabs.

According to some embodiments, the gap is at least six times larger than the thickness of the tabs.

According to some embodiments, the gap is at least eight times larger than the thickness of the tabs.

According to another aspect of the invention, there is provided a prosthetic valve comprising a frame movable between a radially compressed configuration and a radially expanded configuration, a leaflet assembly mounted within the frame, and a plurality of adjustable commissure supports.

The leaflet assembly comprises a plurality of leaflets configured to regulate flow through the prosthetic valve. Each leaflet comprises a rounded cusp end portion defining a cusp edge, a free edge opposite to the cusp edge, and a pair of oppositely-directed tabs separating the cusp edge and the free edge.

Each adjustable commissure support comprises a support base attached to the frame, two adjustable arms coupled to the support base, and rotatable relative thereto, and two scallop adjustment arms. Each adjustable arm comprises an engagement portion. Each scallop adjustment arm extends continuously from one of the adjustable arms.

The tabs of adjacent leaflets are attached to the adjustable commissure supports, such that each tab is attached to one of the adjustable arms. Portions of the cusp edge of each leaflet are attached to scallop adjustment arms of adjustable commissure supports on both sides of the leaflet.

Both adjustable arms and scallop adjustment arms of a single adjustable commissure supports are configured to rotate in opposite directions, when opposite rotational forces are applied to their engagement portions. Rotation of the adjustable arms and the scallop adjustment arms causes the tabs to wind around the adjustable arms they are attached to, and causes the portions of the cusp edges to wind around the scallop adjustment arms they are attached to.

According to some embodiments, the support base is integrally formed with the frame.

According to some embodiments, each adjustable arm and the scallop adjustment arm extending therefrom are integrally formed.

According to some embodiments, the scallop adjustment arm comprises a torque transmitting shaft.

According to some embodiments, the support base comprises two eyelets through which the adjustable arms and/or the scallop adjustment arms extend.

According to some embodiments, the frame comprises eyelets through which the scallop adjustment arms extend.

According to some embodiments, the eyelets are extending radially inward.

According to some embodiments, the eyelets comprises internal threads.

According to some embodiments, the tabs are wrapped over the adjustable arms.

According to some embodiments, each adjustable arm comprises a plurality of apertures.

According to some embodiments, the apertures are axially spaced apart from each other.

According to some embodiments, the tabs are sutured to the adjustable arms via sutures through the tabs and the apertures.

According to some embodiments, the sutures extend through portions of the tab disposed over opposite sides of the adjustment arms.

According to some embodiments, portions of the cusp edges are wrapped over the scallop adjustment arms.

According to some embodiments, each scallop adjustment arm comprises a plurality of apertures.

According to some embodiments, the apertures are axially spaced apart from each other.

According to some embodiments, the tabs are sutured to the scallop adjustment arms via sutures through the portions of the cusp edges and the apertures.

According to some embodiments, the sutures extend through portions of the cusp edge disposed over opposite sides of the scallop adjustment arms.

According to some embodiments, each engagement portion comprises a non-cylindrical socket.

According to some embodiments, each engagement portion comprises a proximally oriented extension formed as a drive-screw coupler.

According to some embodiments, each engagement portion comprises an angled surface.

According to some embodiments, both adjustable arms of a single adjustable commissural support are laterally spaced from each other and define a gap therebetween.

According to some embodiments, two tabs of adjacent leaflets of the leaflet assembly are attached to the adjustable commissure support, such that each tab is wrapped over one of the adjustable arms.

According to some embodiments, both tabs are wrapped over the respective adjustable arms in opposite directions with respect to each other.

According to some embodiments, the gap is at least two times larger than the thickness of the tabs.

According to some embodiments, the gap is at least four times larger than the thickness of the tabs.

According to some embodiments, the gap is at least six times larger than the thickness of the tabs.

According to some embodiments, the gap is at least eight times larger than the thickness of the tabs.

According to another aspect of the invention, there is provided a prosthetic valve comprising a frame movable between a radially compressed configuration and a radially expanded configuration, a leaflet assembly mounted within the frame, at least one adjustable commissure supports, and at least one expansion and locking mechanism.

The leaflet assembly comprises a plurality of leaflets configured to regulate flow through the prosthetic valve. Each leaflet comprises a rounded cusp end portion defining a cusp edge, a free edge opposite to the cusp edge, and a pair of oppositely-directed tabs separating the cusp edge and the free edge.

The adjustable commissure support comprises a clamp coupled, directly or indirectly, to the frame at a first location, and a couple of adjustable arms. The clamp comprises a clamp mid-portion and a couple of opposing side arms continuously extending from the clamp mid-portion. Each adjustable arm extending proximally from one of the side arms. Each adjustable arm comprises an engagement portion.

The expansion and locking mechanism comprises an outer member and an inner member. The outer member comprises an outer member coupling recess, wherein the clamp of the adjustable commissure support is clamped over the outer member coupling recess. The inner member is coupled to the frame at a second location spaced apart from the first location. The inner member extends at least partially into the outer member.

Movement of the inner member in a first direction, relative to the outer member, causes the frame to foreshorten axially and expand radially. The side arms are resiliently expandable away from each other, and are inwardly biased toward each other in the absence of an expanding force applied thereto.

The tabs are attached to the adjustable arms. The adjustable arms are configured to rotate or twist when rotational force is applied to the engagement portions. Rotation or twisting of the adjustable arms causes the tabs attached thereto, to wind around the adjustable arms.

According to some embodiments, the clamp mid-portion comprises an opening, wherein the outer member comprises an outer member fastening extension extending radially outward through the opening, and coupled to the frame at the first location.

According to some embodiments, the clamp mid-portion comprises a commissure support fastening extension, extending radially outward from the clamp mid-portion, and coupled to the frame at the first location.

According to some embodiments, the outer member coupling recess has a depth which is equal to, or greater than, the thickness of the clamp.

According to some embodiments, the outer member coupling recess has a height which is not greater than 110% of the height of the clamp.

According to some embodiments, the tabs are wrapped over the adjustable arms.

According to some embodiments, each adjustable arm has a non-circular cross section.

According to some embodiments, each adjustable arm comprises a plurality of apertures.

According to some embodiments, the apertures are axially spaced apart from each other.

According to some embodiments, the tabs are sutured to the adjustable arms via sutures through the tabs and the apertures.

According to some embodiments, the sutures extend through portions of the tab disposed over opposite sides of the adjustment arms.

According to some embodiments, the engagement portion comprises a non-cylindrical socket.

According to some embodiments, the engagement portion comprises a proximally oriented extension formed as a drive-screw coupler.

According to some embodiments, the engagement portion comprises an angled surface.

According to some embodiments, the adjustable arms are rigidly attached to the side arms.

According to some embodiments, the adjustable arms are integrally formed with the side arms.

According to some embodiments, the adjustable arms are configured to twist between the side arms and the engagement portions, upon application of rotational force to the engagement portions.

According to some embodiments, the adjustable arms comprise a plastically deformable material.

According to some embodiments, the adjustable arms are rotationally coupled to the side arms.

According to some embodiments, the adjustable arms are threadedly coupled to the side arms.

According to some embodiments, each side arm comprises a base threaded portion, wherein each adjustable arm comprises an adjustable arm threaded portion, configured to threadedly engage with the base threaded portion.

According to some embodiments, both adjustable arms of a single adjustable commissural support are laterally spaced from each other and define a gap therebetween.

According to some embodiments, two tabs of adjacent leaflets of the leaflet assembly are attached to the adjustable commissure support, such that each tab is wrapped over one of the adjustable arms.

According to some embodiments, both tabs are wrapped over the respective adjustable arms in opposite directions with respect to each other.

According to some embodiments, the gap is at least two times larger than the thickness of the tabs.

According to some embodiments, the gap is at least four times larger than the thickness of the tabs.

According to some embodiments, the gap is at least six times larger than the thickness of the tabs.

According to some embodiments, the gap is at least six times larger than the thickness of the tabs.

According to some embodiments, the gap, at a free state of the side arms, is smaller than a width of the outer member at the region of the outer member coupling recess.

According to some embodiments, the adjustable arms are offset radially inward with respect to the outer member, defining an offsetting gap therebetween.

According to some embodiments, the offsetting gap is larger than the thickness of the tab.

According to some embodiments, the offsetting gap is at least two times larger than the thickness of the tab.

According to some embodiments, the offsetting gap is at least three times larger than the thickness of the tab.

According to some embodiments, the offsetting gap is at least four times larger than the thickness of the tab.

According to another aspect of the invention, there is provided a delivery assembly comprising a prosthetic valve and a delivery apparatus. The prosthetic valve comprises a frame movable between a radially compressed configuration and a radially expanded configuration, a leaflet assembly mounted within the frame, and a plurality of adjustable commissure supports.

The leaflet assembly comprises a plurality of leaflets configured to regulate flow through the prosthetic valve. Each leaflet comprises a rounded cusp end portion defining a cusp edge, a free edge opposite to the cusp edge, and a pair of oppositely-directed tabs separating the cusp edge and the free edge.

Each adjustable commissure support comprises a support base attached to the frame, and at least one adjustable arm extending proximally from the support base. Each adjustable arm comprises an engagement portion.

The delivery apparatus comprises a handle, a delivery shaft extending distally from the handle, and at least one adjustment assembly extending from the handle through the delivery shaft. The adjustment assembly comprises at least one adjustment arm equipped with a driving head. The driving head is releasably coupled to the engagement portion.

The tabs are attached to the adjustable arms. Rotational force applied to the adjustment arms is configured to rotate the engagement portions therewith, thereby causing the adjustable arms to rotate or twist. Rotation or twisting of the adjustable arms causes the tabs attached thereto, to wind around the adjustable arms.

According to some embodiments, the adjustment arm is a torque transmitting arm, configured to transmit torque from the handle to the engagement portion.

According to some embodiments, each adjustment assembly further comprises an adjustment sleeve extending over the adjustment arm.

According to some embodiments, the adjustment sleeve and the adjustment arms are movable longitudinally relative to each other.

According to some embodiments, the tabs are wrapped over the adjustable arms.

According to some embodiments, each adjustable arm has a non-circular cross section.

According to some embodiments, each adjustable arm comprises a plurality of apertures.

According to some embodiments, the apertures are axially spaced apart from each other.

According to some embodiments, the tabs are sutured to the adjustable arms via sutures through the tabs and the apertures.

According to some embodiments, the sutures extend through portions of the tab disposed over opposite sides of the adjustment arms.

According to some embodiments, the engagement portion comprises a non-cylindrical socket.

According to some embodiments, the driving head comprises a distally oriented non-circular extension, dimensioned to be inserted into the socket.

According to some embodiments, the engagement portion comprises a proximally oriented extension formed as a drive-screw coupler.

According to some embodiments, the engagement portion comprises an angled surface.

According to some embodiments, the driving head comprises a distally oriented extension formed as a drive-screw coupler, complementary to the drive-screw coupler shape of the engagement portion.

According to some embodiments, the adjustable arms are rigidly attached to the support base.

According to some embodiments, the adjustable arms are integrally formed with the support base.

According to some embodiments, the adjustable arms are configured to twist between the support base and the engagement portions, upon application of rotational force to the engagement portions.

According to some embodiments, the adjustable arms comprise a plastically deformable material.

According to some embodiments, the adjustable arms are rotationally coupled to the support base.

According to some embodiments, the adjustable arms are threadedly coupled to the support base.

According to some embodiments, the support base comprises at least one base threaded portion, wherein each adjustable arm comprises an adjustable arm threaded portion, configured to threadedly engage with the base threaded portion.

According to some embodiments, the support base further comprises at least one offsetting extension, extending radially therefrom, wherein the at least one adjustable arm extends from the respective offsetting extension.

According to some embodiments, the at least one offsetting extension extends radially away from the support base.

According to some embodiments, the at least one offsetting extension extends radially inward with respect to the support base.

According to some embodiments, the offsetting extension is integrally formed with the support base.

According to some embodiments, each adjustable commissure support comprises a single adjustable arm.

According to some embodiments, two tabs of adjacent leaflets of the leaflet assembly are attached to the adjustable commissure support, such that both tabs are wrapped over the single adjustable arm.

According to some embodiments, the leaflets are asymmetric leaflets, each asymmetric leaflet having a short tab and an opposite long tab, wherein the tabs of adjacent leaflets are wrapped over the single adjustable arm such that the short tab of one leaflet is wrapped around the adjustable arm, in direct contact therewith, and the long tab of the adjacent leaflet is wrapped over the short tab.

According to some embodiments, each adjustment assembly comprises a single adjustment arm.

According to some embodiments, each adjustable commissure support comprises two adjustable arms that are laterally spaced from each other and define a gap therebetween.

According to some embodiments, two tabs of adjacent leaflets of the leaflet assembly are attached to the adjustable commissure support, such that each tab is wrapped over one of the adjustable arms.

According to some embodiments, both tabs are wrapped over the respective adjustable arms in opposite directions with respect to each other.

According to some embodiments, each adjustment assembly comprises two adjustment arms.

According to another aspect of the invention, there is provided a delivery assembly comprising a prosthetic valve and a delivery apparatus. The prosthetic valve comprises a frame movable between a radially compressed configuration and a radially expanded configuration, a leaflet assembly mounted within the frame, at least one adjustable commissure support, and at least one expansion and locking assembly.

The leaflet assembly comprises a plurality of leaflets configured to regulate flow through the prosthetic valve. Each leaflet comprises a rounded cusp end portion defining a cusp edge, a free edge opposite to the cusp edge, and a pair of oppositely-directed tabs separating the cusp edge and the free edge.

The adjustable commissure support comprises a clamp coupled, directly or indirectly, to the frame at a first location, and a couple of adjustable arms. The clamp comprises a clamp mid-portion and a couple of opposing side arms continuously extending from the clamp mid-portion. Each adjustable arm extending proximally from one of the side arms. Each adjustable arm comprises an engagement portion.

The expansion and locking mechanism comprises an outer member and an inner member. The outer member comprises an outer member coupling recess, wherein the clamp of the adjustable commissure support is clamped over the outer member coupling recess. The inner member is coupled to the frame at a second location spaced apart from the first location. The inner member extends at least partially into the outer member.

The delivery apparatus comprises a handle, a delivery shaft extending distally from the handle, and at least one adjustment assembly extending from the handle through the delivery shaft. The adjustment assembly comprises at least one adjustment arm equipped with a driving head. The driving head is releasably coupled to the engagement portion.

Movement of the inner member in a first direction, relative to the outer member, causes the frame to foreshorten axially and expand radially. The side arms are resiliently expandable away from each other, and are inwardly biased toward each other in the absence of an expanding force applied thereto.

The tabs are attached to the adjustable arms. Rotational force applied to the adjustment arms is configured to rotate the engagement portions therewith, thereby causing the adjustable arms to rotate or twist. Rotation or twisting of the adjustable arms causes the tabs attached thereto, to wind around the adjustable arms.

According to some embodiments, the clamp mid-portion comprises an opening, and the outer member comprises an outer member fastening extension extending radially outward through the opening, and coupled to the frame at the first location.

According to some embodiments, the clamp mid-portion comprises a commissure support fastening extension, extending radially outward from the clamp mid-portion, and coupled to the frame at the first location.

According to some embodiments, the outer member coupling recess has a depth which is equal to, or greater than, the thickness of the clamp.

According to some embodiments, the outer member coupling recess has a height which is not greater than 110% of the height of the clamp.

According to some embodiments, the tabs are wrapped over the adjustable arms.

According to some embodiments, each adjustable arm has a non-circular cross section.

According to some embodiments, each adjustable arm comprises a plurality of apertures.

According to some embodiments, the apertures are axially spaced apart from each other.

According to some embodiments, the tabs are sutured to the adjustable arms via sutures through the tabs and the apertures.

According to some embodiments, the sutures extend through portions of the tab disposed over opposite sides of the adjustment arms.

According to some embodiments, the engagement portion comprises a non-cylindrical socket.

According to some embodiments, the driving head comprises a distally oriented non-circular extension, dimensioned to be inserted into the socket.

According to some embodiments, the engagement portion comprises a proximally oriented extension formed as a drive-screw coupler.

According to some embodiments, the engagement portion comprises an angled surface.

According to some embodiments, the driving head comprises a distally oriented extension formed as a drive-screw coupler, complementary to the drive-screw coupler shape of the engagement portion.

According to some embodiments, the adjustable arms are rigidly attached to the side arms.

According to some embodiments, the adjustable arms are integrally formed with the side arms.

According to some embodiments, the adjustable arms are configured to twist between the side arms and the engagement portions, upon application of rotational force to the engagement portions.

According to some embodiments, the adjustable arms comprise a plastically deformable material.

According to some embodiments, the adjustable arms are rotationally coupled to the side arms.

According to some embodiments, the adjustable arms are threadedly coupled to the side arms.

According to some embodiments, each side arm comprises a base threaded portion, wherein each adjustable arm comprises an adjustable arm threaded portion, configured to threadedly engage with the base threaded portion.

According to some embodiments, both adjustable arms of a single adjustable commissural support are laterally spaced from each other and define a gap therebetween.

According to some embodiments, two tabs of adjacent leaflets of the leaflet assembly are attached to the adjustable commissure support, such that each tab is wrapped over one of the adjustable arms.

According to some embodiments, both tabs are wrapped over the respective adjustable arms in opposite directions with respect to each other.

According to some embodiments, the gap is at least two times larger than the thickness of the tabs.

According to some embodiments, the is at least four times larger than the thickness of the tabs.

According to some embodiments, the gap is at least six times larger than the thickness of the tabs.

According to some embodiments, the gap is at least eight times larger than the thickness of the tabs.

According to some embodiments, the gap, at a free state of the side arms, is smaller than a width of the outer member at the region of the outer member coupling recess.

According to some embodiments, the adjustable arms are offset radially inward with respect to the outer member, defining an offsetting gap therebetween.

According to some embodiments, the offsetting gap is larger than the thickness of the tab.

According to some embodiments, the offsetting gap is at least two times larger than the thickness of the tab.

According to some embodiments, the offsetting gap is at least three times larger than the thickness of the tab.

According to some embodiments, the offsetting gap is at least four times larger than the thickness of the tab.

According to some embodiments, the adjustment arm is a torque transmitting arm, configured to transmit torque from the handle to the engagement portion.

According to some embodiments, each adjustment assembly further comprises an adjustment sleeve extending over the adjustment arm.

According to some embodiments, the adjustment sleeve and the adjustment arms are movable longitudinally relative to each other.

According to another aspect of the invention, there is provided a commissure adjustment assembly, comprising a valve holder and an adjustment handle, releasably attachable to the valve holder. The valve holder comprises annular holder body, configured to accommodate a prosthetic valve therein.

The adjustment handle comprises an annular driver gear, a plurality of pinion gears rotatable by the driver gear, and a plurality of driving rods, wherein each driving rod extends from a corresponding pinion gear. Each driving rod comprises a driving head, configured to engage with an engagement portion of an engagement portion of adjustable arms of the valve.

According to some embodiments, the valve holder comprises at least one recess, wherein the adjustment handle comprises at least one clamp configured to engage with the at least one recess.

According to some embodiments, the adjustment handle further comprises a handle knob, configured to facilitate rotation of the driver gear.

According to some embodiments, the handle knob and the driver gear are integrally formed.

According to some embodiments, the driver gear is an internal gear having internal teeth, and the pinion gears are external gears having external teeth, such that the teeth of at least some of the pinion gears are meshed with the teeth of the driver gear.

According to some embodiments, the pinion gears comprise primary pinion gears meshed with the driver gear.

According to some embodiments, the prosthetic valve positioned within the valve holder, wherein the prosthetic valve comprises a plurality of adjustable commissure supports. Each adjustable commissure support comprises a single adjustable arm having the engagement portion, wherein the driving head of each driving rod extending from a primary pinion gear is engaged with a corresponding engagement portion.

According to some embodiments, the commissure adjustment assembly further comprises the prosthetic valve positioned within the valve holder, wherein the prosthetic valve comprises a plurality of adjustable commissure supports, and wherein each adjustable commissure support comprises two adjustable arm having engagement portions. The driving head of each driving rod extending from a primary pinion gear is engaged with an engagement portion of one adjustable arm of an adjustable engagement support, wherein each two of the plurality of primary pinion gears having their driving rods engaged with two adjustable arms of the same adjustable commissure supports, are not meshed with each other, and are having their driving rods spaced apart from each other by a gap that is equal to the gap between the respective engagement arms they are engaged with.

According to some embodiments, the pinion gears further comprise secondary pinion gears, which are meshed with the primary pinion gears, but not with the secondary pinion gears.

According to some embodiments, the commissure adjustment assembly further comprises the prosthetic valve positioned within the valve holder, wherein the prosthetic valve comprises a plurality of adjustable commissure supports, and wherein each adjustable commissure support comprises two adjustable arm having engagement portions. The driving head of each driving rod extending from a primary pinion gear is engaged with an engagement portion of one adjustable arm of an adjustable engagement support, wherein the driving head of each driving rod extending from the secondary pinion gear meshed with the primary pinion gear is engaged with an engagement portion of the other adjustable arm of the same adjustable engagement support.

According to another aspect of the invention, there is provided a method of assembling a commissure assembly, comprising the steps of: (1) wrapping a tab of one leaflet over a single adjustable arm extending from a support base of an adjustable commissure support of a prosthetic valve; (2) wrapping another tab of an adjacent leaflet over the previous tab; and (3) suturing both tabs to the adjustable arm via a suture extending through both tabs and apertures of the adjustable arm.

According to some embodiments, the step of suturing includes passing the suture through portions of the tabs disposed over opposite sides of the adjustment arm.

According to some embodiments, the step of wrapping a tab of one leaflet over the adjustable arm comprises wrapping a short tab of the one leaflet over the adjustable arm, and wherein the step of wrapping another tab includes wrapping a long tab of the adjacent leaflet over the short tab.

According to another aspect of the invention, there is provided a method of assembling a commissure assembly, comprising the steps of: (1) extending two tabs of two adjacent leaflets through a gap of an adjustable commissure support; (2) wrapping each tab over a respective adjustable arm, wherein both tabs are wrapped in opposite directions with respect to each other; and (3) suturing each tab to the respective adjustable arm via a suture extending through the tab and apertures of the adjustable arm.

According to some embodiments, the step of suturing includes passing the suture through portions of the tab disposed over opposite sides of the adjustment arm.

According to another aspect of the invention, there is provided a method for adjusting the tension of leaflets of a prosthetic valve, comprising the steps of: (1) placing a prosthetic valve having a plurality of adjustable commissure supports, within a valve holder of a commissure adjustment assembly; (2) releasably coupling an adjustment handle to the valve holder, such that driving heads of driving rods extending from pinion gears that are positioned within a driver gear, engage with engagement portions of adjustable arms of the adjustable commissure supports; and (3) rotating the driver gear to facilitate rotation of the pinion gears rotatable thereby, which in turn rotate the engagement portions therewith.

According to some embodiments, the step of placing the prosthetic valve within the valve holder, includes a step of expanding the prosthetic valve against the annular holder body of the valve holder.

According to some embodiments, each adjustable commissure support includes a single adjustable arm, wherein the pinion gears include primary pinion gears meshed with the driver gear. The step of coupling includes engaging the driving heads of the driving rods extending from the primary pinion gears with the engagement portions of the single adjustable arms of the adjustable commissure supports. The step of rotating includes rotating the driver gear to facilitate rotation of the primary pinion gears meshed therewith.

According to some embodiments, each adjustable commissure support includes two adjustable arms, wherein the pinion gears include primary pinion gears meshed with the driver gear, and secondary pinion gears meshed with the primary pinion gears but not with the driver gear. The step of coupling includes engaging the driving head of the driving rod extending from each primary pinion gear with one adjustable arm of an adjustable commissure support, and engaging the driving head of the driving rod extending from the secondary pinion gear meshed with the with the primary pinion gear, with the other adjustable arm of the same adjustable commissure support. The step of rotating includes rotating the driver gear to facilitate rotation of the primary pinion gears and the secondary pinion gears in opposite rotational directions.

According to another aspect of the invention, there is provided a method for adjusting the tension of leaflets of a prosthetic valve, comprising the steps of: (1) expanding a prosthetic valve that includes a plurality of adjustable commissure supports, to a final expanded diameter; (2) rotating adjustment arms that extend from a handle of a delivery apparatus, and are coupled via driving heads thereof to adjustment portions of adjustable arms of the adjustable commissure supports, to facilitate rotation of the engagement portions therewith; and (3) disengaging the driving heads from the adjustment portions.

According to some embodiments, the step of disengaging the driving heads includes pulling the driving heads proximally away from the engagement portions.

According to some embodiments, the step of disengaging the driving heads includes pulling adjustment sleeves that are disposed over the adjustment arms in a proximal direction.

According to some embodiments, the step of disengaging the driving heads includes simultaneously pulling the adjustment arms with adjustment sleeves disposed thereover.

According to some embodiments, the step of rotating the adjustment arms includes rotating each couple of adjustment arms engaged with two adjustable arms that extend from a single support base, in opposite rotational directions.

According to some embodiments, the method further comprises a step of retrieving the delivery apparatus.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the Figures:

FIG. 1 shows a view in perspective of a delivery assembly, according to some embodiments.

FIG. 2 shows a view in perspective of a prosthetic valve, according to some embodiments.

FIG. 3 shows a flattened view of a single leaflet, according to some embodiments.

FIG. 4 shows a leaflet assembly defined by three leaflets, prior to assembly, according to some embodiments.

FIG. 5A shows a top view of a prosthetic valve with an overstretched leaflet assembly mounted therein, according to some embodiments.

FIG. 5B shows a top view of a prosthetic valve with an under-stretched leaflet assembly mounted therein, according to some embodiments.

FIG. 6 shows an exemplary prosthetic valve comprising adjustable commissure supports, according to some embodiments.

FIGS. 7A-7B show enlarged views of a commissure assembly coupled to an adjustable commissure support, prior to and after applying rotational movement to adjustable arms thereof, according to some embodiments.

FIGS. 8A-8B show top views of a prosthetic valve comprising adjustable commissure supports, prior to and after applying rotational movement to adjustable arms thereof, according to some embodiments.

FIGS. 9A-9B show enlarged views of an adjustable commissure support, prior to and after twisting adjustable arms thereof, according to some embodiments.

FIGS. 10A-10B show enlarged views of an adjustable commissure support, prior to and after rotating adjustable arms thereof, according to some embodiments.

FIG. 11 shows a cross-sectional view of a commissure adjustment assembly with a prosthetic valve accommodated therein, according to some embodiments.

FIG. 12 shows a partial view in perspective of an adjustment handle, according to some embodiments,

FIG. 13 shows a cross-sectional view of the adjustment handle of FIG. 12.

FIG. 14 shows an exemplary prosthetic valve comprising adjustable commissure supports equipped with a single adjustable arm, according to some embodiments.

FIGS. 15A-15B show top views of a prosthetic valve comprising adjustable commissure supports, prior to and after applying rotational movement to single adjustable arms thereof, according to some embodiments.

FIG. 16 shows a flattened view of an asymmetric leaflet, according to some embodiments.

FIGS. 17A-17B show top views of a prosthetic valve comprising asymmetric leaflets, prior to and after applying rotational movement to adjustable arms attached thereto, according to some embodiments.

FIG. 18 shows a cross-sectional view of a commissure adjustment assembly with a prosthetic valve accommodated therein, according to some embodiments.

FIG. 19 shows a partial view in perspective of an adjustment handle, according to some embodiments,

FIG. 20 shows a cross-sectional view of the adjustment handle of FIG. 19.

FIG. 21 shows a view in perspective of a delivery assembly equipped with adjustment assemblies, according to some embodiments.

FIGS. 22A-22C show steps of manipulating an adjustable commissure support via an adjustment assembly, according to some embodiments.

FIGS. 23A-23C show steps of manipulating an adjustable commissure support via an adjustment assembly, according to additional embodiments.

FIG. 24 shows an exemplary prosthetic valve comprising adjustable commissure supports having offsetting portions extending radially outward, according to some embodiments.

FIG. 25 shows an exemplary prosthetic valve comprising adjustable commissure supports having offsetting portions extending radially inward, according to some embodiments.

FIG. 26 shows an exemplary prosthetic valve comprising adjustable commissure supports having scallop adjustment arms, according to some embodiments.

FIG. 27 shows the prosthetic valve of FIG. 26, with a leaflet assembly coupled to the adjustable commissure supports, according to some embodiments.

FIG. 28 shows a view in perspective of a delivery assembly carrying a mechanically expandable valve, according to some embodiments.

FIG. 29A shows a view in perspective of a mechanically expandable valve, according to some embodiments.

FIG. 29B shows a view in perspective of an inner member of an expansion and locking assembly, according to some embodiments.

FIG. 29C shows a view in perspective of an expansion and locking assembly, according to some embodiments.

FIGS. 30A-30C show an expansion and locking assembly of the type shown in FIG. 29C in different operational states thereof.

FIG. 31 shows a view in perspective of an adjustable commissure support having a clamp, according to some embodiments.

FIG. 32 shows an outer member of an expansion and locking assembly, provided with a coupling recess for accommodating a clamp of an adjustable commissure support, according to some embodiments.

FIG. 33 shows an expansion and locking assembly coupled to a frame of a prosthetic valve, while an adjustable commissure support is clamped thereto, according to some embodiments.

FIG. 34 shows a view in perspective of an adjustable commissure support equipped with adjustable arms that are rotatably attachable to its clamp, according to some embodiments.

FIG. 35 shows a view in perspective of an adjustable commissure support having a fastening extension, according to some embodiments.

FIGS. 36A-36B shows the adjustable commissure support coupled to a frame, such that its adjustable arms are positioned radially inward and radially outward relative to the frame, respectively, according to some embodiments.

FIG. 37 shows a view in perspective of an adjustable commissure support having a fastening extension, wherein its adjustable arms are rotatably attachable to its support base, according to some embodiments.

FIG. 38 shows a view in perspective of a delivery assembly equipped with adjustment assemblies and actuation assemblies, according to some embodiments.

FIGS. 39A-39E show an expansion and locking assembly and an adjustable commissure support clamped thereto, in different operational states thereof.

### DETAILED DESCRIPTION OF SOME EMBODIMENTS

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples. In view of the many possible embodiments to which the principles of the disclosed technology may be applied, it should be recognized that the illustrated embodiments are only preferred examples and should not be taken as limiting the scope of the disclosed technology.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the terms “have” or “includes” means “comprises.” As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

Throughout the figures of the drawings, different superscripts for the same reference numerals are used to denote different embodiments of the same elements. Embodiments of the disclosed devices and systems may include any combination of different embodiments of the same elements. Specifically, any reference to an element without a superscript may refer to any alternative embodiment of the same element denoted with a superscript. In order to avoid undue clutter from having too many reference numbers and lead lines on a particular drawing, some components will be introduced via one or more drawings and not explicitly identified in every subsequent drawing that contains that component.

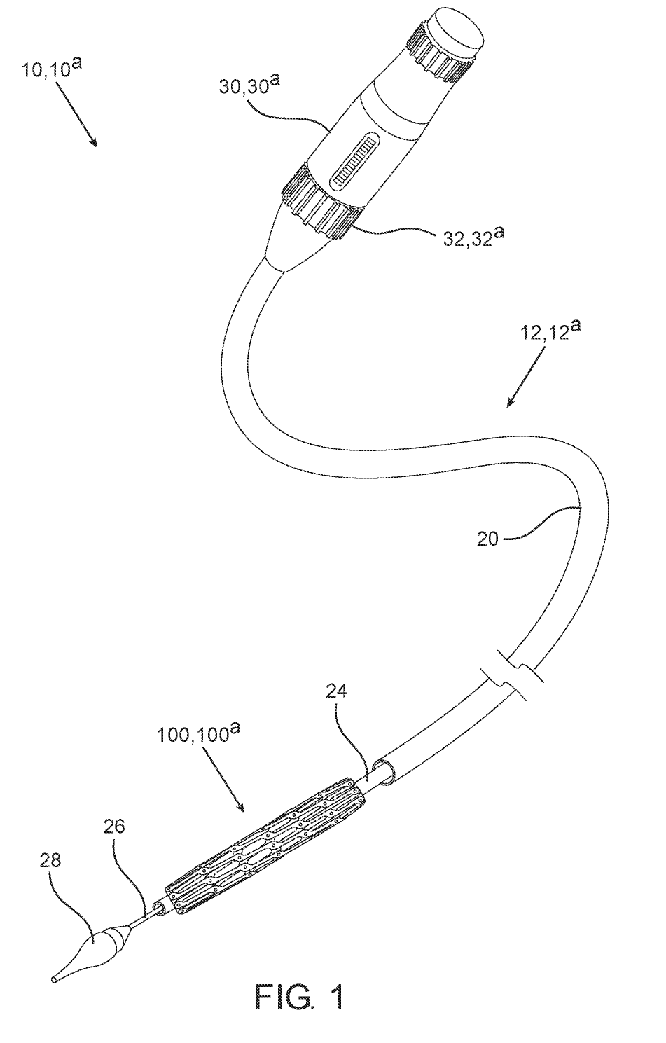

FIG. 1 shows a view in perspective of a delivery assembly 10, according to some embodiments. The delivery assembly 10 can include a prosthetic valve 100 and a delivery apparatus 12. The prosthetic valve 100 can be on or releasably coupled to the delivery apparatus 12. The delivery apparatus can include a handle 30 at a proximal end thereof, a nosecone shaft 26 extending distally from the handle 30, and a nosecone 28 attached to the distal end of the nosecone shaft 26.

The term “proximal”, as used herein, generally refers to the side or end of any device or a component of a device, which is closer to the handle 30 or an operator of the handle 30 when in use.

The term “distal”, as used herein, generally refers to the side or end of any device or a component of a device, which is farther from the handle 30 or an operator of the handle 30 when in use.

The term “prosthetic valve”, as used herein, refers to any type of a prosthetic valve deliverable to a patient’s target site over a catheter, which is radially expandable and compressible between a radially compressed, or crimped, state, and a radially expanded state. Thus, a prosthetic valve 100 can be crimped or retained by a delivery apparatus 12 in a compressed state during delivery, and then expanded to the expanded state once the prosthetic valve 100 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

The term “plurality”, as used herein, means more than one.

A prosthetic valve 100 of the current disclosure may include any prosthetic valve configured to be mounted within the native aortic valve, the native mitral valve, the native pulmonary valve, and the native tricuspid valve. While a delivery assembly 10 described in the current disclosure, includes a delivery apparatus 12 and a prosthetic valve 100, it should be understood that the delivery apparatus 12 according to any embodiment of the current disclosure can be used for implantation of other prosthetic devices aside from prosthetic valves, such as stents or grafts.

A catheter deliverable prosthetic valve 100 can be delivered to the site of implantation via the delivery assembly 10 carrying the valve 100 in a radially compressed or crimped state, toward the target site, to be mounted against the native anatomy, by expanding the prosthetic valve 100 via various expansion mechanisms. Balloon expandable valves generally involve a procedure of inflating a balloon within a prosthetic valve, thereby expanding the prosthetic valve 100 within the desired implantation site. Once the valve is sufficiently expanded, the balloon is deflated and retrieved along with the delivery apparatus 12. Self-expandable valves include a frame that is shape-set to automatically expand as soon an outer retaining capsule, which may be also defined as the distal portion of an outer shaft (20) or the distal portion of a delivery shaft, is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies, releasably coupled to respective actuation assemblies of the delivery apparatus 12, controlled via the handle 30 for actuating the expansion and locking assemblies to expand the prosthetic valve to a desired diameter. The expansion and locking assemblies may optionally lock the valve’s diameter to prevent undesired recompression thereof, and disconnection of the actuation assemblies from the expansion and locking assemblies, to enable retrieval of the delivery apparatus 12 once the prosthetic valve is properly positioned at the desired site of implantation.

The delivery assembly 10 can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

The exemplary delivery assembly 10 illustrated in FIG. 1 may be a delivery assembly 10^{a} comprising a delivery apparatus 12^{a} for delivery and implantation of a balloon expandable valve 100^{a}. According to some embodiments, the delivery apparatus 12^{a} includes a balloon catheter 24 having an inflatable balloon (hidden from view) mounted on its distal end. The balloon expandable prosthetic valve 100^{a} can be carried in a crimped state over the inflatable balloon, as shown in FIG. 1. Optionally, an outer shaft 20 can concentrically extend over the balloon catheter 24.

The outer shaft 20 and the balloon catheter 24 can be configured to be axially movable relative to each other, such that a proximally oriented movement of the outer shaft 20 relative to the balloon catheter 24, or a distally oriented movement of the balloon catheter 24 relative to the outer shaft 20, can expose the prosthetic valve 100^{a} from the outer shaft 20.

The proximal ends of the balloon catheter 24, and when present - the outer shaft 20, can be coupled to the handle 30^{a}. During delivery of the prosthetic valve 100^{a}, the handle 30^{a} can be maneuvered by an operator (e.g., a clinician or a surgeon) to axially advance or retract components of the delivery apparatus 12^{a}, such as the nosecone shaft 26, the balloon catheter 24, and/or the outer shaft 20, through the patient’s vasculature, as well as to inflate the balloon mounted on the balloon catheter 24, so as to expand the prosthetic valve 100^{a}, and to deflate the balloon and retract the delivery apparatus 12^{a} once the prosthetic valve 100^{a} is mounted in the implantation site.

According to some embodiments, the handle 30 can include one or more operating interfaces, such as steerable or rotatable adjustment knobs 32, levers, sliders, buttons and other actuating mechanisms, which are operatively connected to different components of the delivery apparatus 12 and configured to produce axial movement of components of the delivery apparatus 12 in the proximal and distal directions, as well as to expand or contract the prosthetic valve 100 via various mechanisms. For example, a handle 30^{a} can include rotatable or otherwise maneuverable knobs 32^{a} for axially moving the nosecone shaft 26, the balloon catheter 24, the outer shaft 20, and/or to inflate and deflate the inflatable balloon.

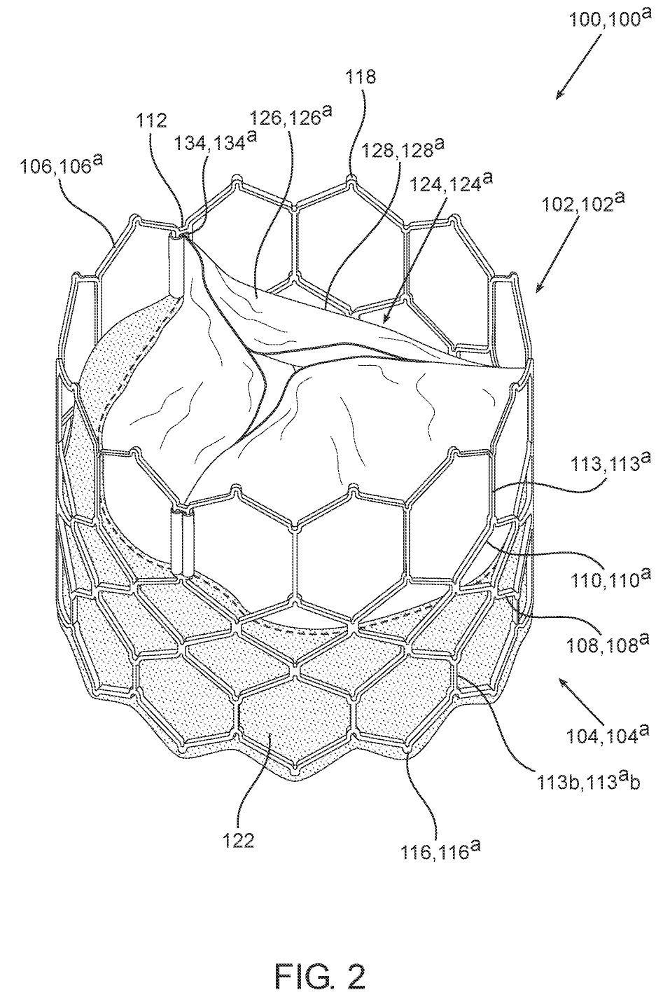

FIG. 2 shows an exemplary prosthetic valve 100 in an expanded state, according to some embodiments. The prosthetic valve 100 can comprise an inflow end 104 and an outflow end 102. In some instances, the outflow end 102 is the distal end of the prosthetic valve 100, and the inflow end 104 is the proximal end of the prosthetic valve 100. Alternatively, depending for example on the delivery approach of the valve, the outflow end can be the proximal end of the prosthetic valve, and the inflow end can be the distal end of the prosthetic valve.

The term “outflow”, as used herein, refers to a region of the prosthetic valve through which the blood flows through and out of the valve 100, for example between the valve’s central longitudinal axis and the outflow end 102.

The term “inflow”, as used herein, refers to a region of the prosthetic valve through which the blood flows into the valve 100, for example between inflow end 104 and the valve’s central longitudinal axis.

The valve 100 comprises an annular frame 106 movable between a radially compressed configuration and a radially expanded configuration, and a leaflet assembly 124 mounted within the frame 106. The frame 106 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 106 can be crimped to a radially compressed state on a balloon catheter 24, and then expanded inside a patient by an inflatable balloon or equivalent expansion mechanism. Alternatively or additionally, the frame 106 can be made of shape-memory materials such as, but not limited to, nickel titanium alloy (e.g., Nitinol). When constructed of a shape-memory material, the frame 106 can be crimped to a radially compressed state and restrained in the compressed state by insertion into a shaft or equivalent mechanism of a delivery apparatus 12.

In the example illustrated in FIG. 2, the frame 106 is an annular, stent-like structure comprising a plurality of intersecting struts 110. In this application, the term “strut” encompasses vertical struts, angled struts, attachment posts, commissure windows, and any similar structures described by U.S. Pat. Nos. 7,993,394 and 9,393,110, which are incorporated herein by reference. A strut 110 may be any elongated member or portion of the frame 106. The frame 106 can have one or more multiple rows of cells 108 defined by intersecting struts 110. The frame 106 can have a cylindrical or substantially cylindrical shape having a constant diameter from the inflow end 104 to the outflow end 102 of the frame as shown, or the frame can vary in diameter along the height of the frame, as disclosed in U.S. Pat. No. 9,155,619, which is incorporated herein by reference.

The end portions of the struts 110 are forming apices 118 at the outflow end 102 and apices 116 at the inflow end 104. The struts 110 can intersect at additional junctions 114 formed between the outflow apices 118 and the inflow apices 116. The junctions 114 can be equally or unequally spaced apart from each other, and/or from the apices 118, 116, at the outflow end 102 and the inflow end 104.

According to some embodiments, the struts 110 include a plurality of angled struts and axial struts (113). FIG. 2 shows an exemplary embodiment of a type of a prosthetic valve 100^{a}, that can be representative of, but is not limited to, a balloon expandable prosthetic valve. The frame 106^{a} of the prosthetic valve 100^{a} illustrated in FIG. 2 comprises a plurality of angled struts 110^{a}, and axially extending struts 113^{a}. In such embodiments, the struts may be pivotable or bendable relative to each other, so as to permit frame expansion or compression. For example, the frame 106^{a} can be formed from a single piece of material, such as a metal tube, via various processes such as, but not limited to, laser cutting, electroforming, and/or physical vapor deposition, while retaining the ability to collapse/expand radially in the absence of hinges and like.

The leaflet assembly 124 comprises a plurality of leaflets 126 (e.g., three leaflets), positioned at least partially within the frame 106, and configured to regulate flow of blood through the prosthetic valve 100 from the inflow end 104 to the outflow end 102. While three leaflets 126 arranged to collapse in a tricuspid arrangement, are shown in the exemplary embodiment illustrated in FIG. 1, it will be clear that a prosthetic valve 100 can include any other number of leaflets 126. The leaflets 126 are made of a flexible material, derived from biological materials (e.g., bovine pericardium or pericardium from other sources), biocompatible synthetic materials, or other suitable materials as known in the art and described, for example, in U.S. Pat. Nos. 6,730,118, 6,767,362 and 6,908,481, which are incorporated by reference herein.

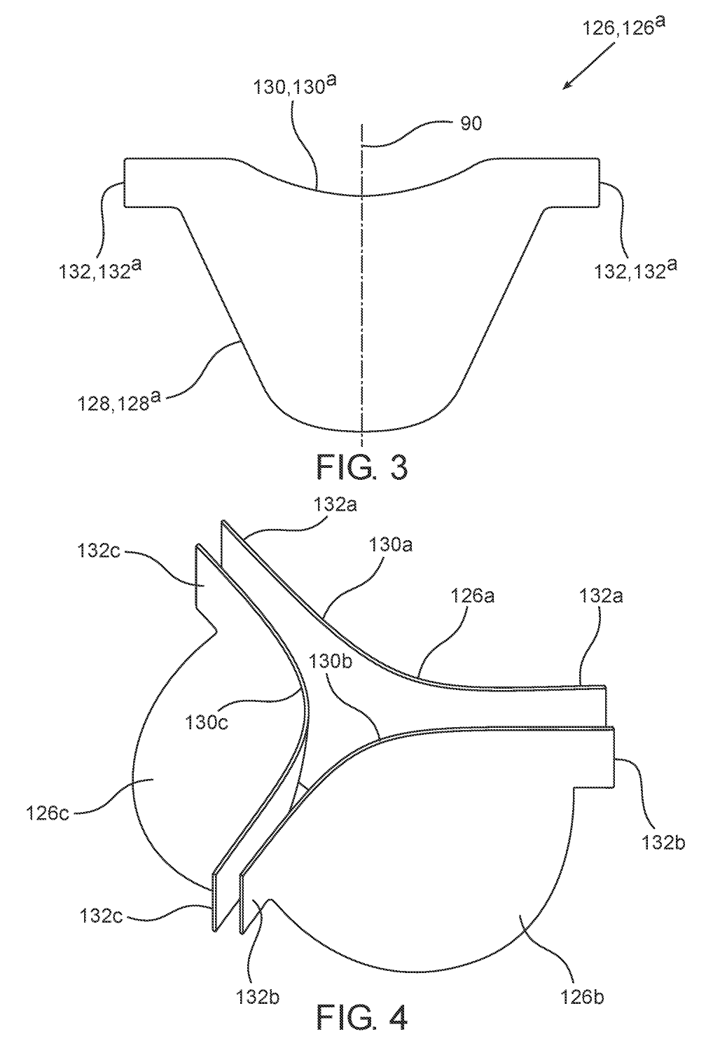

FIG. 3 shows a single representative leaflet 126, and FIG. 4 shows three separated leaflets 126a, 126b and 126c, prior to attachment to the frame 106 and to each other, collectively defining the leaflet assembly 124, according to some embodiments. Each leaflet 126 has a rounded cusp edge 128 opposite a free edge 130, and a pair of generally oppositely-directed tabs 132 separating the cusp edge 128 and the free edge 130. The cusp edge 128 forms a single scallop.

When the leaflets 126 are coupled to the frame and to each other, the lower edge of the resulting leaflet assembly 124 desirably has an undulating, curved scalloped shape. By forming the leaflets with this scalloped geometry, stresses on the leaflets 126 are reduced which, in turn, improves durability of the valve. Moreover, by virtue of the scalloped shape, folds and ripples at the belly of each leaflet, which can cause early calcification in those areas, can be eliminated or at least minimized. The scalloped geometry also reduces the amount of tissue material used to form the leaflet structure, thereby allowing a smaller, more even crimped profile at the inflow end of the valve.

A conventional leaflet 126, such as leaflet 126^{a} shown in FIG. 3, is symmetrical with respect to a leaflet central axis 90 extending vertically along it’s center (in a flattened state of the leaflet), such that if the leaflet 126 is theoretically folded over itself along the leaflet central axis 90, both tabs 132 are congruent with respect to each other.

The leaflets 126 define a non-planar coaptation plane (not annotated) when their free edges 130 co-apt with each other to seal blood flow through the prosthetic valve 100. Leaflets 126 can be secured to one another at their tabs 132 to form commissure assemblies 134 of the leaflet assembly 124, which can be secured, directly or indirectly, to structural elements connected to the frame 106 or embedded therein, such as commissure posts or commissure windows. When secured to two other leaflets 126 to form leaflet assembly 124, the cusp edges 128 of the leaflets 126 collectively form the scalloped shaped lower edge portion of the leaflet assembly 124.

According to some embodiments, such as the exemplary embodiment illustrated in FIG. 2 for a prosthetic valve 100^{a}, the upper row of cells comprises a plurality of axially extending window frame portions 112 (which define commissure windows) and a plurality of axially extending struts 113^{a}a. Additional axially extending struts 113^{a}b along the lower row of cells is shown by way of illustration and not limitation. Each axial strut 113^{a}a and each axially extending window frame portion 112 can extend from a junction 114^{a} defined by the convergence of the lower ends of two angled struts 110^{a} to another junction 114^{a} defined by the convergence of the upper ends of two angled struts 110^{a}. Each axially extending window frame portion 112 mounts a respective commissure assembly 134^{a} of the leaflet assembly 124^{a}.

Additional frame configurations may include commissure posts attached to the frame, configured to accept commissure assemblies 134 extending either through window portions defined therein, or supporting commissure assembly attachment thereto in various other manners. Alternatively, some of the cells 108 may be configured to receive commissure assemblies 134 attached thereto. For example, the uppermost row of cells 108 can be configured to receive tabs 132 of the leaflets 126. Further details regarding prosthetic valves, including the manner in which commissures may be mounted to their frames, are described in U.S. Pat. Nos. 6,730,118, 7,393,360, 7,510,575, 7,993,394, 8,252,202, and 9,393,110; U.S. Publication Nos. 2018/0325665, 2019/0105153, U.S. Application Nos. 62/869,948 and 62/813,643; and PCT Application No. PCT/US2019/61392, all of which documents are incorporated herein by reference. Any of the techniques and mechanisms disclosed in the prior documents can be used to connect commissure assemblies 134, directly or indirectly, to the frame 106.

According to some embodiments, the prosthetic valve 100 further comprises an inner skirt 122 that can be secured to the inner surface of the frame 106. The inner skirt 122 is configured to assist in securing the leaflet assembly 124 to the frame 106 and to assist in forming a good seal between the prosthetic valve (100) and the native annulus by blocking the flow of blood through the open cells 108 of the frame (106) below the cusp edges 128 of the leaflets (126). The leaflets 126 may be sutured to the inner skirt 122 via a scallop-line suture that tracks their lower cusp edges 128, while the inner skirt 122 can be sutured to the frame 106 at locations away from the scallop-line suture so that the inner skirt 122 can be more pliable in that area. This can avoid stress concentrations at the scallop-line suture.

According to some embodiments, the prosthetic valve 100 can further comprise an outer skirt (not shown) mounted on the outer surface of the frame 106, configure to function, for example, as a sealing member retained between the frame 106 and the surrounding tissue of the native annulus against which the prosthetic valve 100 is mounted, thereby reducing risk of paravalvular leakage past the prosthetic valve 100. Either one of the inner skirt (122) or the outer skirt can be made of various suitable biocompatible materials, such as, but not limited to, various synthetic materials (e.g., PET) or natural tissue (e.g. bovine pericardium).

The leaflets 126 define a non-planar coaptation plane (not annotated) when their free edges 130 coapt with each other to seal blood flow through the prosthetic valve 100. In some implementations, the free edge 130 of a leaflet 126 can extend along a straight line between the tabs 132. In other implementations, as shown in FIG. 3, the free edge 130^{a} between the tabs 132^{a} is concave with either one or more curvatures (i.e., simple or complex curves).

During diastole, the adjacent free edges 130 should coapt with each other during diastole, to prevent retrograde blood from flowing between the free edges 130. During systole, the adjacent free edges 130 will separate from each other and allow antegrade blood to flow between free edges 130 and help wash away blood from the areas underneath the commissure assemblies 134.

While described and shown throughout the figures with respect to the free edges 130 of adjacent leaflets 126, it is to be understood that any reference within the current specification, to coaptation between leaflets 126, is not limited to the free edges 130 contacting each other, but may also refer to other regions of the leaflets 126, which can be adjacent and distal to the free edges 130, for example, that contact each other in a closed position (i.e., during diastole) in a manner that will prevent retrograde blood flow through the prosthetic valve (100).

Some types of prosthetic valves are adapted for use in a range of expansion diameters, instead of being configured to be usable for a specific single expansion diameter. For example, one type of prosthetic valves can be sized for implantation in a range of diameters from 23 to 26 mm, while another type of the of prosthetic valves can be sized for implantation in a range of diameters from 26 to 29 mm. While the frame (106) can be easily expandable, and potentially lockable, in a relatively wide range of expansion diameters, the coaptation between the leaflets (126) is less flexible, such that over-expansion or under-expansion of the frame may result in impaired coaptation between the leaflets 126.

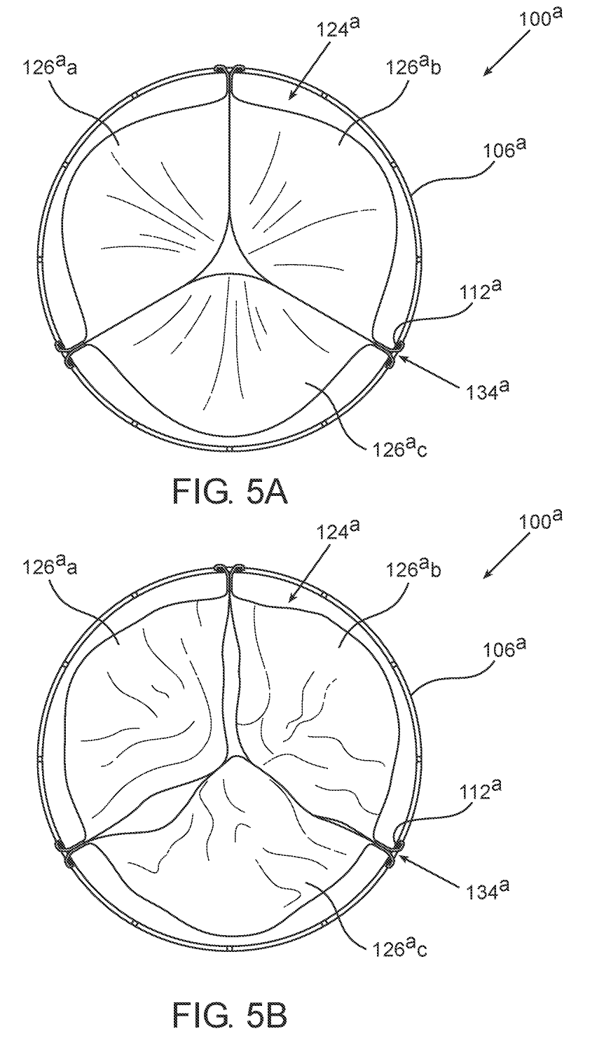

FIGS. 5A and 5B schematically show, from a top view of a prosthetic valve 100^{a}, two exemplary states of impaired coaptation. In FIG. 5A, the leaflets 126^{a} are overstretched, such that during diastole, a gap is formed between at least some of the leaflets 126^{a}, for example at the central portion of the prosthetic valve (100^{a}). This situation may result from a frame (106^{a}) which is expanded beyond a maximal diameter at which the leaflets 126^{a} were designed to properly coapt.

In FIG. 5B, an opposite situation is shown, in which the leaflets 126^{a} were designed to properly coapt at an expansion diameter which is larger than the actual diameter attained by the frame 106^{a}, resulting in loose slacks of under-stretched leaflets that form undesirable gaps between adjacent leaflets (126^{a})

Disclosed herein are prosthetic valves that include adjustable commissure supports, configured to adjust the length of the free edges between the commissural assemblies attached to the adjustable commissure supports, so as to facilitate adequate coaptation between the leaflets in a chosen diameter or a chosen range of diameters.

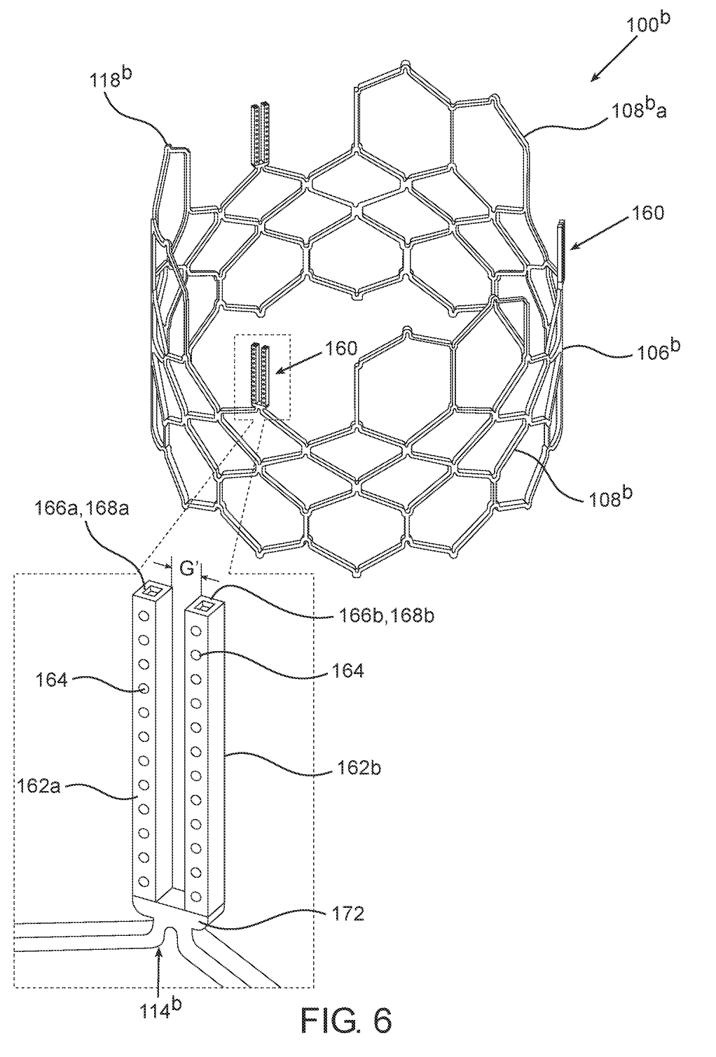

FIG. 6 shows an exemplary prosthetic valve 100^{b} comprising an adjustable commissure support 160, in accordance with some applications of the present invention. An adjustable commissure support 160 can be formed as an integral component of the frame 106, or a component attachable to the frame 106. An adjustable commissure support 160 includes at least one adjustable arm 162, to which at least one leaflet tab 132 can be attached, wherein the adjustable arm 162 is configured to rotate or twist about its central axis, thereby winding the leaflet tab 132 attached thereto onto the adjustable arm 162. The valve 100^{b} is shown without the soft components, such as leaflets or skirts, for clarity.

The adjustable commissure support 160 may further include support base 172, from which the at least one adjustable arm 162. Preferably, the at least one adjustable arm 162 is vertically oriented, parallel to a longitudinal axis of the valve. The at least one adjustable arm 162 preferably extends proximally from the support base 172.

According to some embodiments, the adjustable commissure support 160 comprises two adjustable arms 162a and 162b, circumferentially or laterally spaced apart from each other so as to define a gap G′ there-between, as shown in FIG. 6. Both adjustable arms 162 extend proximally from a support base 172, which is dimensioned to define the gap G′ between the adjustable arms 162. This configuration can define a commissural window, configured to attach to a commissural assembly (134).

According to some embodiments, the frame 106^{b} comprises a plurality of adjustable commissure supports 160, configured to accommodate a plurality of commissure assemblies (134). While three adjustable commissure supports 160 are illustrated, it will be understood that any other number of adjustable commissure supports 160 is contemplated.

According to some embodiments, the adjustable commissure supports 160 are at the level of the upper (i.e., proximal) row of cells 108^{b}a. According to some embodiments, the upper row of cells 108^{b}a includes axially extending struts (113), and the adjustable commissure supports 160 are positioned between cells 108^{b} with axially extending struts (113), wherein the adjustable arms 162 are parallel to the axially extending struts (113). According to some embodiments, the cells defined on both sides of each adjustable commissure supports 160 are open cells (108), that are open ended at their proximal end, and are thereby devoid of outflow apices.

According to some embodiments, the support base 172 is integrally formed with the frame 106. According to some embodiments, the support base 172 is formed as an integral portion of a junction 114 (as illustrated in FIG. 6), while in other embodiments the support base 172 can be formed as a separate component fixedly attached to a junction.

Each adjustable arm 162 comprises an engagement portion 166, preferably positioned at a proximal end thereof. The engagement portion 166 is configured to non-rotatably engage with an external component, such that rotation of the external component around its axis will serve to rotate or twist the adjustable arm 162 in the same direction.

According to some embodiments, the engagement portion 166 comprises a non-cylindrical socket 168, configured to accommodate a tool therein, to facilitate rotation or twisting of the adjustable arm 162. Sockets 168a and 168b are illustrated as rectangular sockets by way of illustration and not limitation. A socket 168 can have any of various shapes provided with at least one facet, so as to facilitate rotation thereof when a tool or component accommodated therein, having a complementary shape, is rotated.

A socket 168 can be in the form of a screw-drive socket, having at least one facet. In some implementations, a socket 168 can be in the form of a Phillips or a Fearson screw-drive socket. In some implementations, a socket 168 can be in the form of a hex or an Allen screwhead socket. In some implementations, a socket 168 can be in the form of a screw driver socket such as, but not limited to: Slot, Square, Robertson, Torx, TA, Tri-Wing, Clutch, Spanner-Head, Double-Square, Triple-Square, Double-Hex, Bristol and the like.

While illustrated as a socket 168 in FIG. 6, it is to be understood that other types of engagement portions are contemplated. For example, the proximal portion of the adjustable arm 162 can be formed to be insertable into a socket of an external tool or component, which is provided with a non-circular socket implemented according to any of the options described hereinabove for socket 168. For example, a tool with a square-shaped socket can accommodate a proximal end of an adjustable arm 162, which is formed with a complementary shape, having at least one facet.

The adjustable arms 162 illustrated in FIG. 6, for example, are shown as uniform rods having a square (or other polygonal) cross-sectional shape, an external tool or component, having a cup-shaped distal portion with a similarly square-shaped socket can cover the proximal end of such an adjustable arm, such that rotational movement of the external tool or component will rotate the proximal end of the adjustable arm 162 therewith. Thus, the adjustable arms 162 shown in FIG. 6 can be alternatively provided without the sockets 168, wherein the external polygonal shape of their proximal ends can serve as the engagement portions 166.

According to some embodiments, the adjustable commissure supports 160 do not extend proximally beyond the level of the outflow end 102. According to some embodiments, the adjustable commissure supports 160 do not extend proximally beyond the level of the outflow apices 118.

According to some embodiments, the adjustable commissure supports 160 is configured to accommodate a commissural assembly 134. The at least one adjustable arm 162 is configured to attach with at least one tab 132 of a leaflet 126, such that the at least one tab 132 will be wound around the adjustable arm 162 when the adjustable arm 162 is rotated or twisted about its central axis.

According to some embodiments, the adjustable arm 162 comprises a plurality of apertures 164, that can be axially spaced apart from each other along at least a portion of the length of the adjustable arm 162, as shown in FIG. 6.

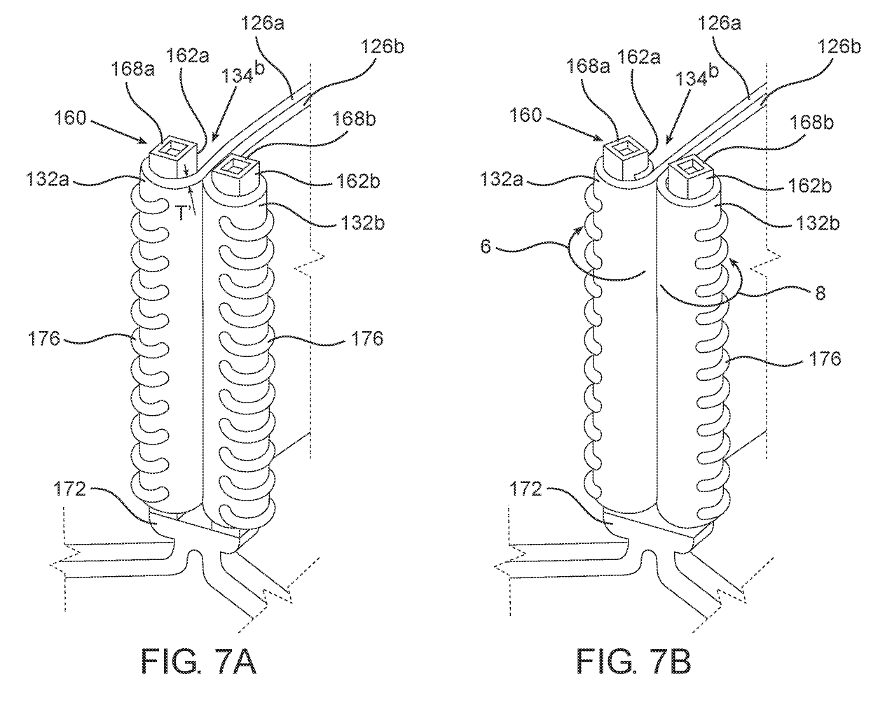

FIG. 7A shows an enlarged view of a commissure assembly 134^{b} coupled to an adjustable commissure support 160, such that each of the tabs 132a and 132b of adjacent leaflets 126a and 126b, is wrapped over and attached to a respective adjustable arm 162a and 162b. FIG. 7A shows an initial step of the tabs 132 attached to the adjustable commissure support 160, prior to being adjusted thereby.

As further shown in FIG. 7A, each tab 132 can be sutured to a respective adjustable arm 162, by extending a suture 176 passing through the tab 132 and the apertures 164 of the adjustable arm 162. Thus, the apertures 164 are preferably dimensioned so as to accommodate a suture 176 extending therethrough, for attachment of the tab 132 to the adjustable arm 162.

Each leaflet 126, and more specifically, tabs 132 of the leaflet (126), can have a thickness T′ (see FIG. 7A). According to some embodiments, the gap G′ is larger than twice the tab thickness T′, so as to accommodate both tabs 132a and 132b extending therethrough.

As further shown in FIG. 7A, each of the tabs 132 extends from the remainder of the leaflet 126 radially outward through the gap G′, and is wrapped around a respective adjustable arms 162 such that each of the tabs 132a and 132b is wrapped in an opposite direction relative to each other. In some embodiments, the sutures 176 can extends through portions of the tabs 132 disposed over opposite sides of the adjustable arm 162.

According to some embodiments, there is provided a method for assembling a commissure assembly (134), comprising steps of: (1) extending two tabs 132 of two adjacent leaflets 126 through a gap G′ of an adjustable commissure support 160; (2) wrapping each tab 132 over a respective adjustable arm 162, wherein both tabs 132 are wrapped in opposite directions with respect to each other; and (3) suturing each tab 132 to the adjustable arm 162 via a suture 176 extending through the tab 132 and apertures 164 of the adjustable arm 162. In some embodiments, the step of suturing includes passing the suture 176 through portions of the tab 132 disposed over opposite sides of the adjustable arm 162.

FIG. 7B shows the state of the commissure assembly 134^{b} attached to the adjustable commissure support 160 of FIG. 7A, during or after adjustment thereof. As shown, each of the adjustable arms 162 is rotated or twisted in an opposite direction. For example, one adjustment arm 162a can be rotated in a first rotational direction (e.g., clockwise), while the other adjustable arm 162b can be rotated in a second rotational direction, opposite to the first rotational direction (e.g., counterclockwise).

While the tabs 132a and 132b are shown to be wrapped around the adjustable arms 162a and 162b in opposite directions, it is to be understood that in alternative embodiments, both tabs 132a and 132b may be wrapped around the adjustable arms 162a and 162b, respectively, in the same direction. In such embodiments, both of the adjustable arms 162a and 162b are rotated in the same circumferential direction, instead of being rotated in opposite direction as shown in FIGS. 7A-7B.

The tabs 132 are attached to the respective adjustable arms 162 in a manner that prevents them from rotationally sliding over the adjustable arms 162, such that when the adjustable arms are rotated or twisted, the tabs 132 are further wrapped around the adjustable arms.

It is to be understood that the tabs 132 can be attached to the adjustable arms 162 in other manners except suturing, such as gluing and any other means of attachment known in the art, as long as the attachment between the tabs 132 and the adjustable arms 162 is performed in such a manner that when an adjustable arms 162 is rotated or twisted, the respective tab 132 attached thereto is wound thereover.

In some implementations, the adjustable arms 162 can have a non-circular cross-section, such as the rectangular cross-section shown in FIGS. 6-7B. It is to be understood that other cross-sectional shapes are contemplated, such as triangular, octagonal, trapezoid and the like. In some implementations, the adjustment arms can have a circular cross-section. However, non-cylindrical shapes of the adjustable arm 162 may improve engagement between the adjustable arm 162 and the tab, ensuring that the tab will further wrap around the adjustable arm 162 as it is rotated or twisted.

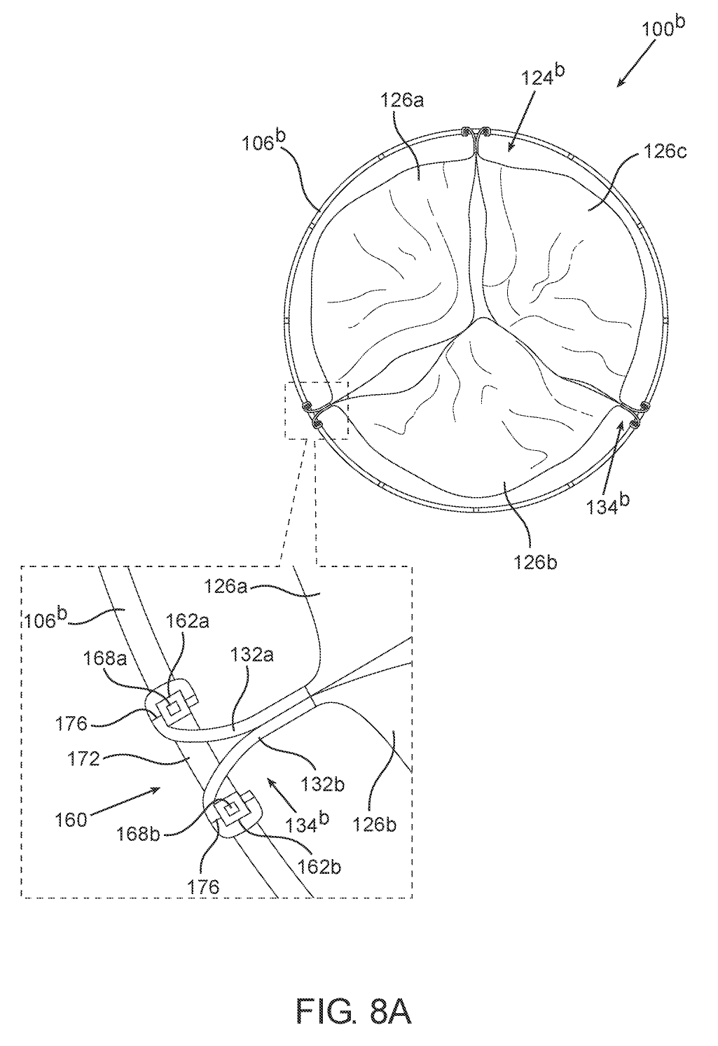

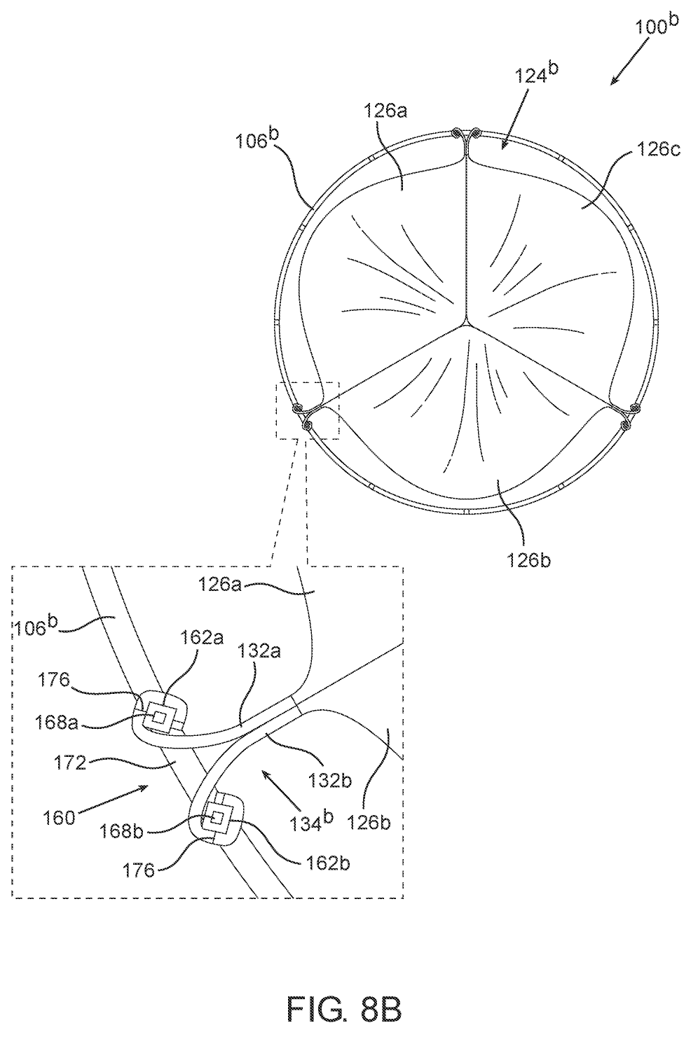

FIG. 8A shows a top view of a prosthetic valve 100^{b} having a leaflet assembly 124^{b} mounted within its frame 106^{b}, prior to leaflets adjustment. In the exemplary configuration shown in FIG. 8A, the frame 106^{b} is expanded to a diameter which is shown to be smaller than the diameter for which the leaflets 126 are designed to properly coapt, thereby material slacks may form undesirable gaps between adjacent leaflets 126.

FIG. 8B shows a state of leaflets adjustment, similar to that shown in FIG. 7B, wherein both adjustable arms 162 are rotated in opposite rotational directions, for example via an external tool or component engaged with their engagement portions 166. The tabs 132 of the leaflets 126 are wound around the adjustable arms 162 during their rotational movement, which tensions the leaflets 126 between their respective commissure assemblies 134^{b}, such that the lengths of their free edges 130 are shortened. Tensioning the leaflets 126 thus may result in proper coaptation therebetween, as shown in FIG. 8B, in the desired expansion diameter of the frame 106^{b}.

Applying too much tension to the leaflets 126, by over-rotating the adjustable arms 162, may result in an over-tensioned state as shown in FIG. 5A. Thus, rotation of the adjustable arms 162 is executed so as to achieve proper coaptation matching the desired expansion diameter, or range of expansion diameters. Rotation of the adjustable arms 162 in an opposite direction, for example - rotating the adjustment arm 162a in a second rotational direction (e.g., counterclockwise), while the other adjustable arm 162b is rotated in a first rotational direction, (e.g., clockwise), can unwind the tabs 132 and release tension from the leaflets 126, that is -elongating the length of the free edges 130 between the commissure assemblies 134^{b}, to release over-tensioned leaflets, or adjust their dimensions to properly coapt in larger expansion diameters of the frame 106^{b}.

The process of proper adjustment of the leaflets 126 may include numerous repeating steps of tensioning and un-tensioning the leaflets, by applying rotational movement to the adjustable arms 162 in the proper rotational directions corresponding to either tensioning or un-tensioning the leaflets 126.

In some implementations, the adjustable arms 162 can be rotated less than 360 degrees. In some implementation, the adjustable arms 162 can be rotated more than 360 degrees. In some cases, rotation of the adjustable arms 162 causes the tabs 132 to be wound over the adjustable arms 162 to such an extent that more than one layer of the tab extends through the gap G′, as shown for example in FIG. 7B. The gap G′ is therefore chosen to accommodate the number of tab layers that can extend therethrough, along the entire expected range of optional rotation of the adjustable arms 162.

According to some embodiments, the gap G′ is at least four times larger than the thickness T′ of the tabs 132. According to some embodiments, the gap G′ is at least six times larger than the thickness T′ of the tabs 132. According to some embodiments, the gap G′ is at least eight times larger than the thickness T′ of the tabs 132.

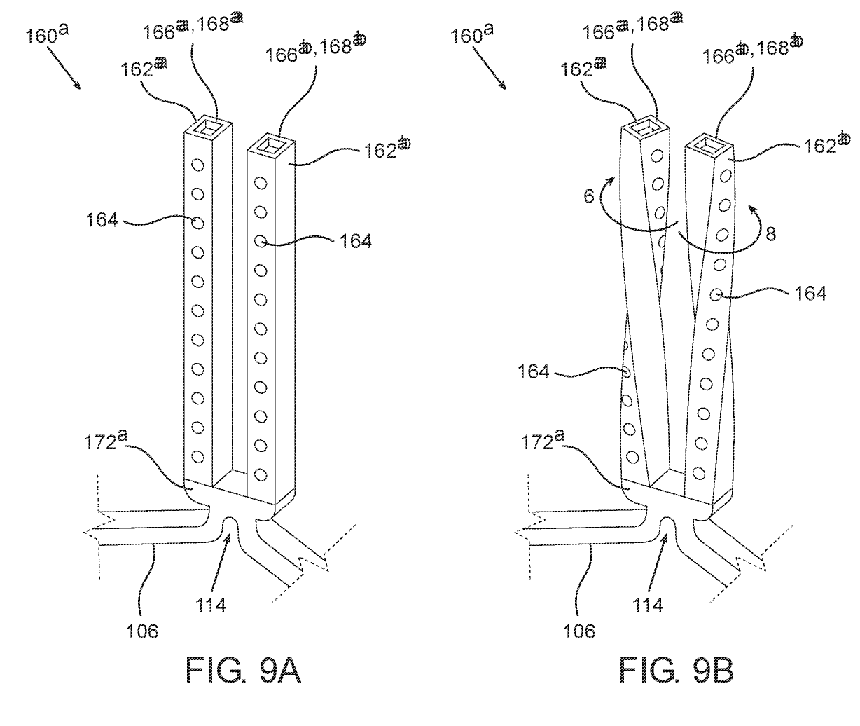

According to some embodiments, the adjustable arms 162 are configured to twist about their central axes, with respect to the support base 172. FIG. 9A shows an adjustable commissure support 160^{a} comprising a couple of adjustable arms 162^{a} which are rigidly attached to the support base 172^{a}. In some embodiments, the adjustable arms 162^{a} are integrally formed with the support base 172^{a}.

As shown in FIG. 9B, when rotational force is applied to the engagement portions 166^{a}, the adjustable arms 162^{a} twist along their length, between the support base 172^{a} and the engagement portions 166^{a}. For example, applying rotational force in a first rotational direction 6 to the engagement portion 166^{a}a, and in some embodiments, to the socket 168^{a}a, while applying rotational force in an opposite, second rotational direction 8 to the engagement portion 166^{a}b, and in some embodiments, to the socket 168^{a}b, will twist the adjustable arms 162^{a}a and 162^{a}b in the opposite directions 6 and 8, respectively. The degree of twisting is chosen to wind the tabs (132) attached to the adjustable arms 162^{a} sufficiently, so as to apply tension to the leaflets (126) that will improve their coaptation.

According to some embodiments, the adjustable arms 162^{a} comprise a plastically deformable material, such that once they assume the twist configuration shown in FIG. 9B, they will not revert back to the untwisted configuration of FIG. 9A, even in the absence of further forces applied to the engagement portions 166^{a}.

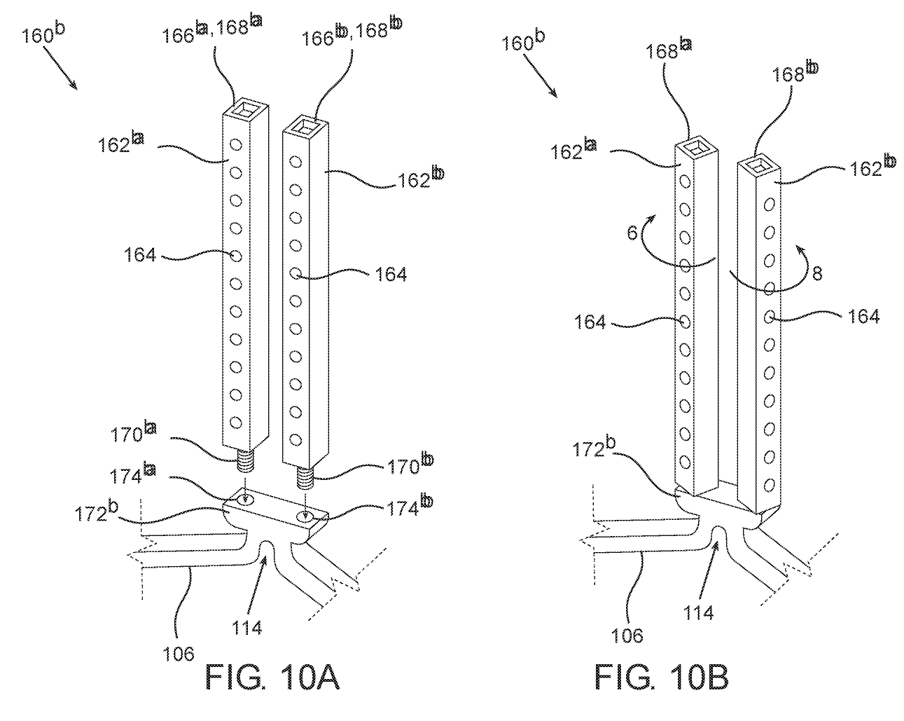

According to some embodiments, the adjustable arms 162 are configured to rotate about their central axes, with respect to the support base 172. FIG. 10A shows an adjustable commissure support 160^{b} comprising a couple of adjustable arms 162^{b} which are rotationally coupled to the support base 172^{b}.

According to some embodiments, the adjustable arms 162^{b} are threadedly coupled the support base 172^{b}. As shown in FIG. 10A, each adjustable arm 162^{b} can include an adjustable arm threaded portion 170^{b}, for example position at its distal end, opposite to the engagement portion 166^{b}. The support base (172) includes at least one base threaded portion 174, and in the case of the support base 172^{b} configured to support two adjustable arms 162^{b}a and 162^{b}b extending therefrom, it includes two base threaded portions 174^{a}a and 174^{a}b.

In the example shown in FIG. 10A, the adjustable arm threaded portion 170^{b} are formed as distally oriented extensions with external threads, configured to engage with base threaded portions 174^{b} formed as internally threaded bores. However, it will be clear that in alternative designs, the base threaded portions can be formed as proximally oriented extensions with external threads, and the adjustable arm threaded portion can be provided as complementary internally threaded bores.

As shown in FIG. 10B, when rotational force is applied to the engagement portions 166^{b}, the adjustable arms 162^{b} are rotated about their central axes with respect to the support base 172^{b}. For example, applying rotational force in a first rotational direction 6 to the engagement portion 166^{b}a, and in some embodiments, to the socket 168^{b}a, while applying rotational force in an opposite, second rotational direction 8 to the engagement portion 166^{b}b, and in some embodiments, to the socket 168^{b}b, will rotate the adjustable arms 162^{a}a and 162^{a}b in the opposite directions 6 and 8, respectively. The amount of rotation is chosen to wind the tabs (132) attached to the adjustable arms 162^{b} sufficiently, so as to apply tension to the leaflets (126) that will improve their coaptation.

In some implementations of the current invention, adjustment of the leaflet assembly (124) mounted within the prosthetic valve (100) is performed prior to implantation into the patient’s body. For example, the desired expansion diameter of the valve (100) can be based on acquired anatomical data of the patient’s anatomy at the target implantation site, which can be achieved via various imaging modalities known in the art, such as pre-CT that is conventionally performed prior to initiating an implantation procedure.

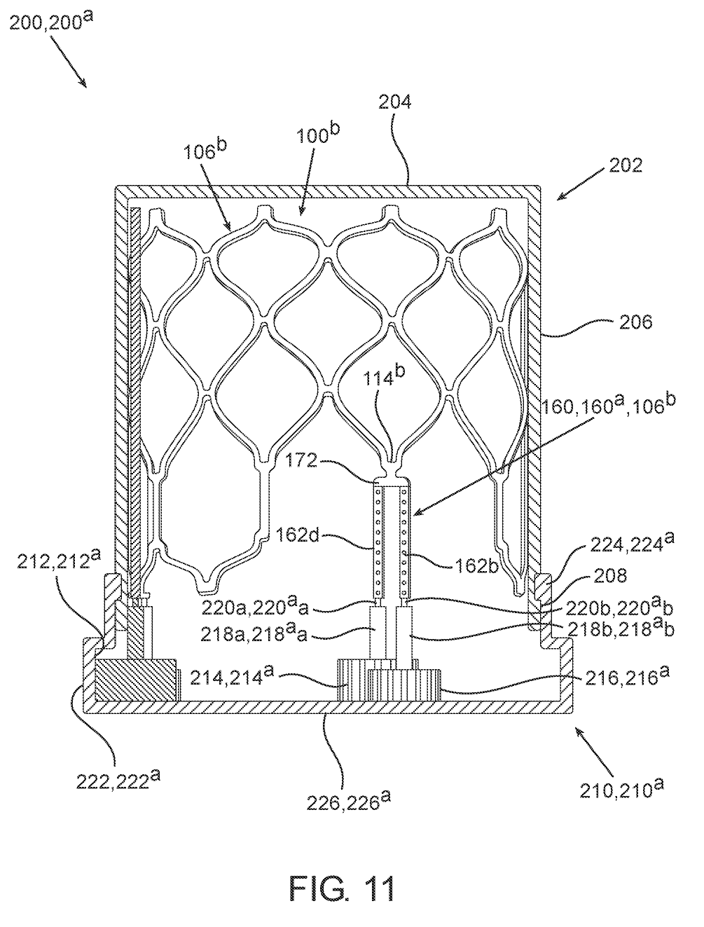

FIG. 11 shows a cross-sectional view of a commissure adjustment assembly 200, according to some applications of the current invention. A commissure adjustment assembly 200 can be utilized to adjust the tension on the leaflets (126) of a prosthetic valve (100) via the adjustable commissure support (160) outside the patient’s body, prior to mounting the valve (100) on a delivery apparatus (12). A commissure adjustment assembly 200 comprises a valve holder 202 configured to accommodate a prosthetic valve 100 therein, and an adjustment handle 210 configured to couple with the adjustable commissure support 160.

The valve holder 202 may be a cup-shaped holder, comprising an annular holder body 206, and optionally a holder base 204. The holder body is dimensioned to accommodate a prosthetic valve 100 in a specific configuration of the valve, such as an expanded or partially expanded configuration. An optional holder base 204 can support the inflow end 104 of the valve (100).

The adjustment handle 210 comprises an annular driver gear 212 and a plurality of pinion gears rotatable by the driver gear 212, wherein a driving rod 218 extends from each pinion gear toward a corresponding adjustable arm 162 of the prosthetic valve 100 accommodated within the valve holder 202. Each driving rod 218 includes a driving head 220 configured to engage with an engagement portion 166 of a corresponding adjustable arm 162.

The driving heads 220 are shaped with complementary shapes to those of the engagement portions 166. For example, if the engagement portion 166 of each adjustable arm 162 comprises a socket 168, the driving head 220 is shaped as a non-cylindrical extension configured to be inserted into the socket 168, such that rotation of the driving head 220 will rotate the engagement portion 166 therewith. More specifically, if the socket 168 is shaped as a rectangular socket, as shown in FIGS. 6-10B, for example, the driving head 220 will can be shaped as a rectangular extension, dimensioned for insertion thereof into the respective socket 168.

The adjustment handle 210 can include handle knob 222, which can be provided in the form of a rotatable knob, configured to facilitate rotation of the driver gear 212. The handle knob 222 can be configured to be maneuverable by an operator of the commissure adjustment assembly 200, for example by grasping the handle knob 222 and manually rotating it. In some embodiments, the handle knob 222 and the driver gear 212 are integrally formed as a single component. In alternative embodiments, the handle knob 222 and the driver gear 212 are separate components, operatively coupled to each other.

The driver gear 212 can be provided as an internal gear, wherein the pinion gears are positioned within the driver gear 212. The driver gear 212 can have internal teeth, while the pinion gears include external teeth, wherein the external teeth of at least some of the pinion gears are meshed with the internal teeth of the driver gear.

According to some embodiments, the adjustment handle 210 is releasably attached to the valve holder 202. For example, the valve holder may include a holder recess 208 (or a plurality or recesses) along the its exterior surface, and the adjustment handle 210 can include complementary handle clamps 224 configured to engage with the holder recess 208. It is to be understood that various releasable attachment mechanisms may be implemented instead of clamps 224 and recesses 208, such as threaded-engagements, snap-fit, frictional engagements, and the like.

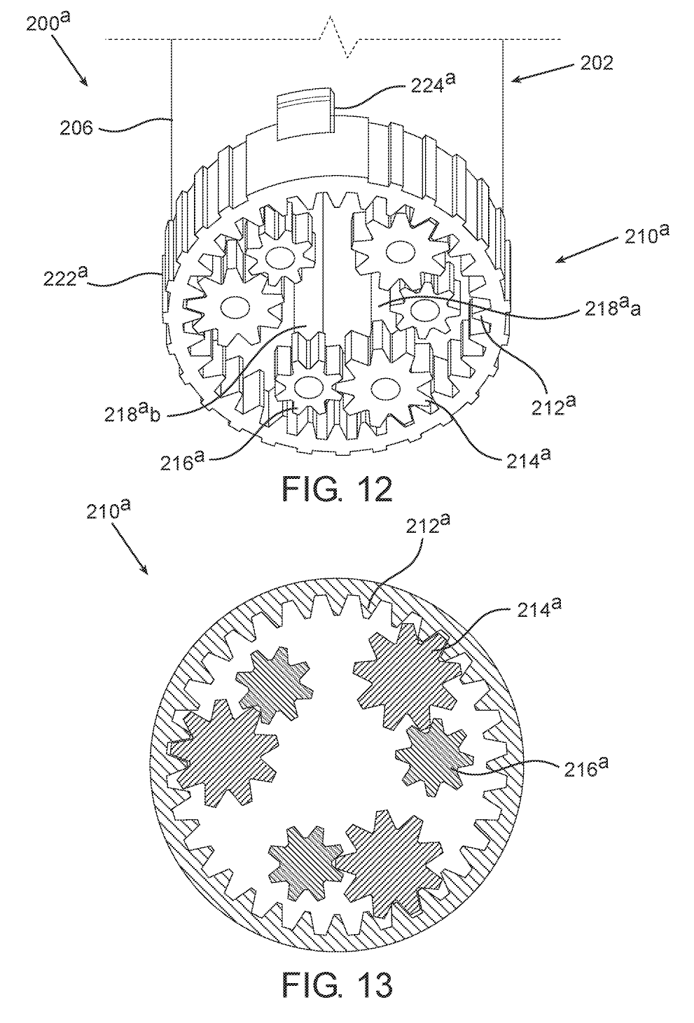

FIG. 11 shows an exemplary type of a commissure adjustment assembly 200^{a}, structured for use with prosthetic valve (100) equipped with adjustable commissure supports (160) that have two adjustable arms 162 extending from each support base 172, according to some applications of the current invention. FIGS. 12 and 13 show a partial view in perspective and a cross-sectional view of an adjustment handle 210^{a}, according to some applications of the current invention.

An adjustment handle 210^{a} can comprise a plurality of pinion gears, that include primary pinion gears 214^{a} and secondary pinion gears 216^{a}. The primary pinion gears 214^{a} are meshed with the driver gear 212^{a}, while the secondary pinion gears 216^{a} are meshed with the primary pinion gears 214^{a}, but not with the driver gear 212^{a}. In this manner, when the driver gear 212^{a} is rotated, it facilitates rotation of the primary pinion gears 214^{a} in one direction, while the secondary pinion gears 216^{a} are rotated by the primary pinion gears 214^{a} in an opposite direction.

The driving rod 218^{a} extending from a primary pinion gear 214^{a} is configured to engage, via its driving head 220^{a}, with one adjustable arm 162a of an adjustable commissure support 160, while the driving rod 218^{a} extending from a secondary pinion gear 216^{a} meshed with the same primary pinion gear 214^{a} is configured to engage, via its driving head 220^{a}, with the other adjustable arm 162b of the same adjustable commissure support 160. In this manner, when each primary pinion gear 214^{a} and a secondary pinion gear 216^{a} meshed therewith are rotated in opposite rotational directions, they rotate the engagement portions 166a and 166b of two adjustable arms 162 of each adjustable commissure support 160 in opposite rotational directions, which can facilitate twisting of both adjustable arms 162^{a}a and 162^{a}b, or rotation of both adjustable arms 162^{b}a and 162^{b}b, in opposite directions with respect to each other, as shown in FIGS. 9B or 10B, respectively.

As mentioned above, both tabs 132a and 132b maybe wrapped around the adjustable arms 162a and 162b in the same direction, in some embodiments, instead of in opposite directions. In such embodiments, the commissure adjustment assembly 200 will be modified to include only primary pinion gears 214 which are meshed directly with the driver gear 212 but not with each other, and wherein the number of primary pinion gears 214 matches the number of adjustable arms 162. For example, if the prosthetic valve 100 includes three adjustable commissure supports 160, each having two adjustable arms 162 extending from its support base 172, the total number of primary pinion gears 214 can be six, including three couples of primary pinion gears 214 wherein each couple includes two pinion gears 214 that are spaced apart from each other such that the primary pinion gears 214 are not meshed with each other, and the driving rods 218 extending therefrom are spaced from each other by a gap G′, enabling their driving heads 220 to engage with the corresponding engagement portions 166. In this manner, rotation of the driver gear 212 will rotate all of the primary pinion gears 214 in the same direction, which will translate the rotational movement to the engagement portions 166 of all respective adjustable arms 162.

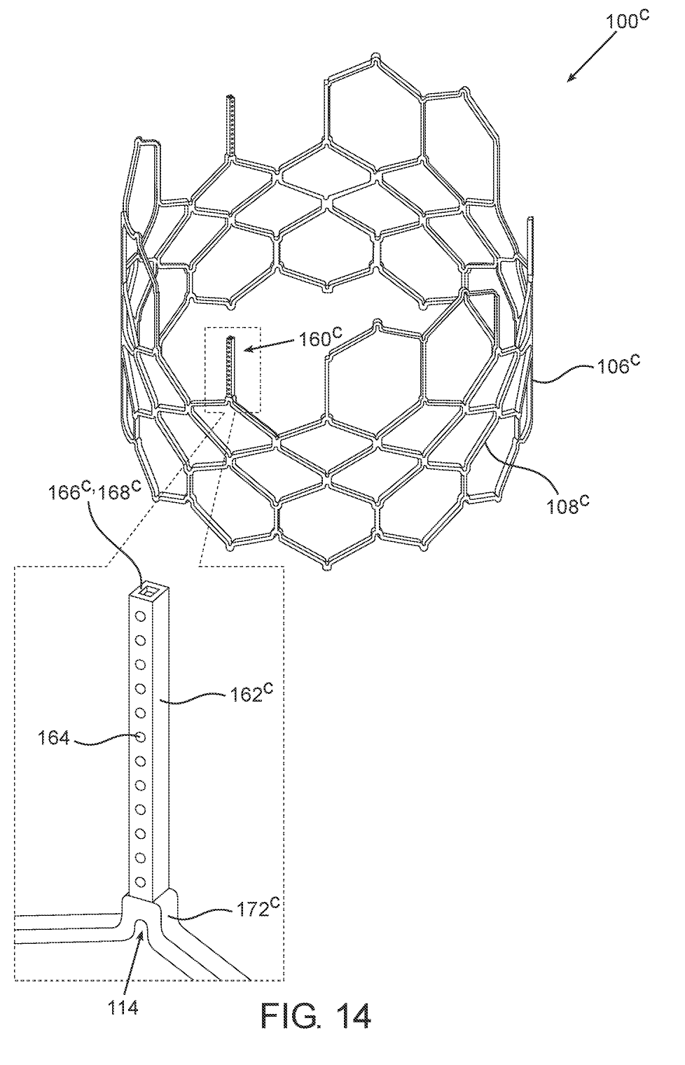

FIG. 14 shows an exemplary prosthetic valve 100^{c} comprising an adjustable commissure support 160^{c}, in accordance with some applications of the present invention. An adjustable commissure support 160^{c} includes a single adjustable arm 162^{c}, extending from the support base 172^{c}. The adjustable arm 162^{c} can be identical to any of the adjustable arms 162 described hereinabove with respect to FIG. 6-10B, including any of the embodiments described for adjustable arms 162^{a} or 162^{b}. The support base 172^{c} can be similar to any support base 172 described hereinabove with respect to FIG. 6-10B, including any of the embodiments described for support base 172^{a} or 172^{b}, except that support base 172^{c} can be dimensioned to support a single adjustable arm 162^{c} extending therefrom, instead of two. Similarly, the frame 106^{c} of the prosthetic valve 100^{c} can be similar to any of the embodiments described hereinabove for 106^{b} of the prosthetic valve 100^{b}, mutatis mutandis.

It is to be understood that any of the features described with respect to adjustable commissure supports 160^{a} or 160^{b} can be implemented in combination with the single adjustable arm 162^{c} of the adjustable commissure support 160^{c}. For example, the adjustable arm 162^{c} can be formed as a twistable arm comprising a plastically deformable material, in a manner similar to that described for adjustable arms 162^{a} with respect to FIGS. 9A-9B. Likewise, the adjustable arm 162^{c} can be rotatably engaged with the support base 172^{c}, via an adjustment arm threaded portion (170^{c}) threaded with a corresponding base threaded portion 174^{c}, in a manner similar to that described for adjustable arms 162^{b} with respect to FIGS. 10A-10B.

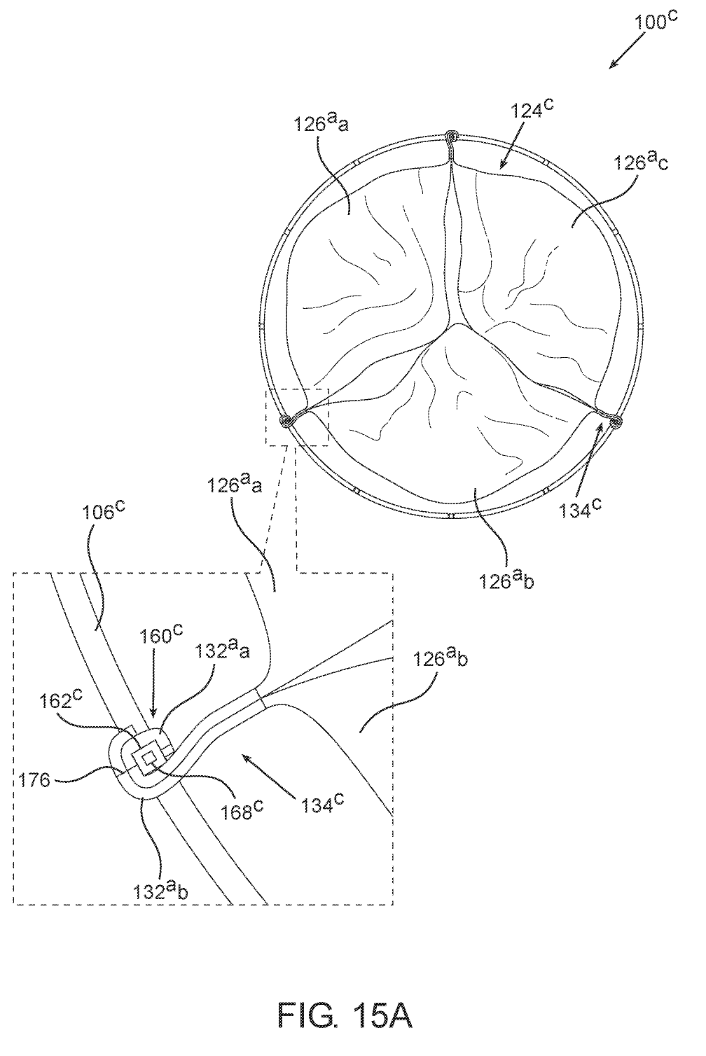

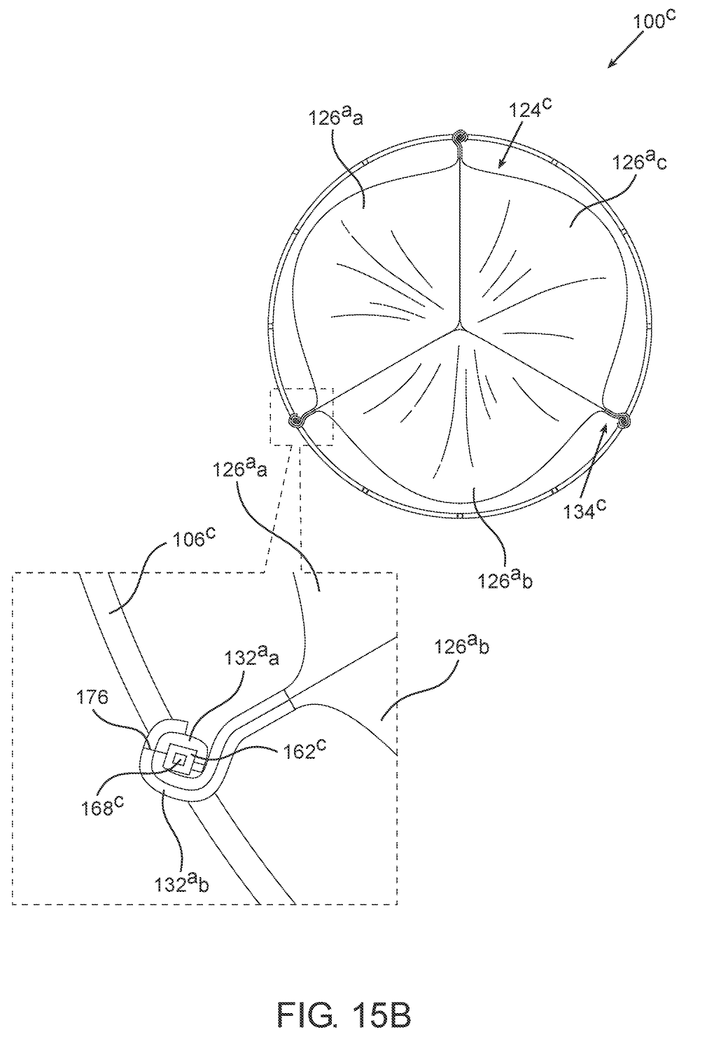

FIG. 15A shows a top view of a prosthetic valve 100^{c} having a leaflet assembly 124^{c} mounted within its frame 106^{c}, prior to leaflets adjustment. In the exemplary configuration shown in FIG. 15A, similar to the configuration of FIG. 8A, the frame 106^{b} is expanded to a diameter which is shown to be smaller than the diameter for which the leaflets 126^{a} are designed to properly coapt, thereby material slacks may form undesirable gaps between adjacent leaflets 126^{a}. FIG. 15B shows a state of leaflets adjustment, similar to that shown in FIG. 15B, wherein the adjustable arms 162^{c} are rotated or twisted, for example via an external tool or component engaged with their engagement portions 166^{c}.

As shown in the zoomed-in region of FIGS. 15A-15B, both tabs 132^{a}a and 132^{a}b of two adjacent leaflets 126^{a}a and 126^{a}b, respectively, are wrapped over the same single adjustable arm 162^{c}, for example by being sutured thereto via suture 176. When the adjustable arm 162^{c} is rotated or twisted, both tabs 132^{a}a and 132^{a}b are wound it, which in turn, which tensions the leaflets 126^{a} between their respective commissure assemblies 134^{c}, such that the lengths of their free edges 130^{a} are shortened.

Conventional leaflets 126^{a}, as shown in FIG. 3 for example, are symmetrical with respect to the leaflet central axis 90, such that the length of both tabs 132^{a} on both sides of the leaflet 126^{a} are identical. Attaching such leaflet tabs 132^{a} to a single adjustable arm 162^{c} requires tab 132^{a}a to be wrapped around the adjustable arm 162^{c}, in direct contact therewith, while the other tab 132^{a}b is wrapped over the previous tab 132^{a}a. This may result in both tabs 132^{a} terminating at different points, as shown in the zoomed-in region of FIGS. 15A-15B.

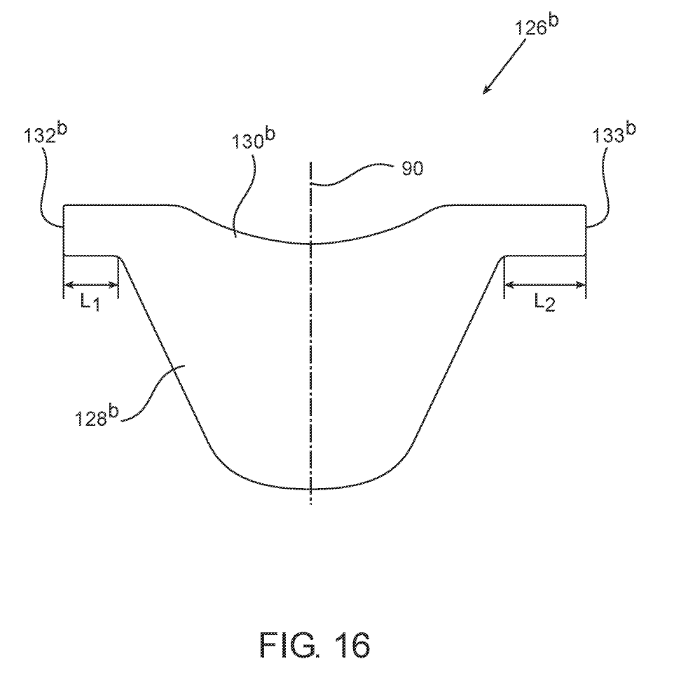

According to some embodiments, the leaflet (126) is asymmetric with respect to the leaflet central axis 90, such that the lengths of the tabs on both sides of the leaflet are different. FIG. 16 shows an exemplary asymmetric leaflet 126^{b}, in accordance with some applications of the current invention. The leaflet 126^{b} has two tabs with different lengths, such as a short tab 132^{b} having a length L₁, and a long tab 133^{b} having a length L₂, wherein L₁ and L₂ are not identical.

According to some embodiments, the length of the long tab L₂ is longer than the length of the short tab L₁. According to some embodiments, L₂ is at least as long as 110% of L₁. According to some embodiments, L₂ is at least as long as 120% of L₁. According to some embodiments, L₂ is at least as long as 150% of L₁. According to some embodiments, L₂ is at least as long as 200% of L₁.

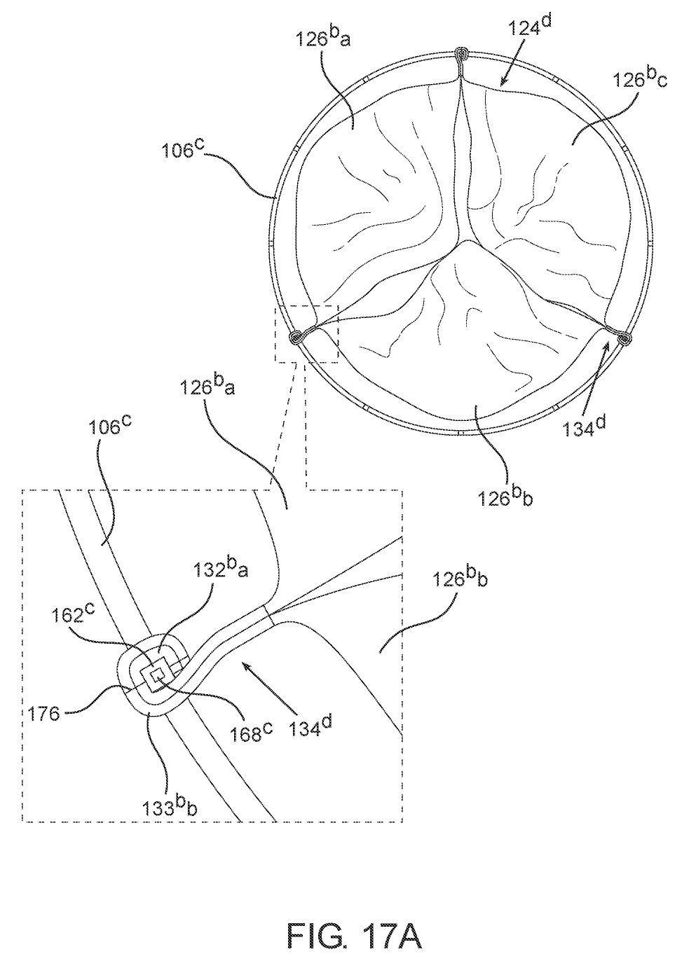

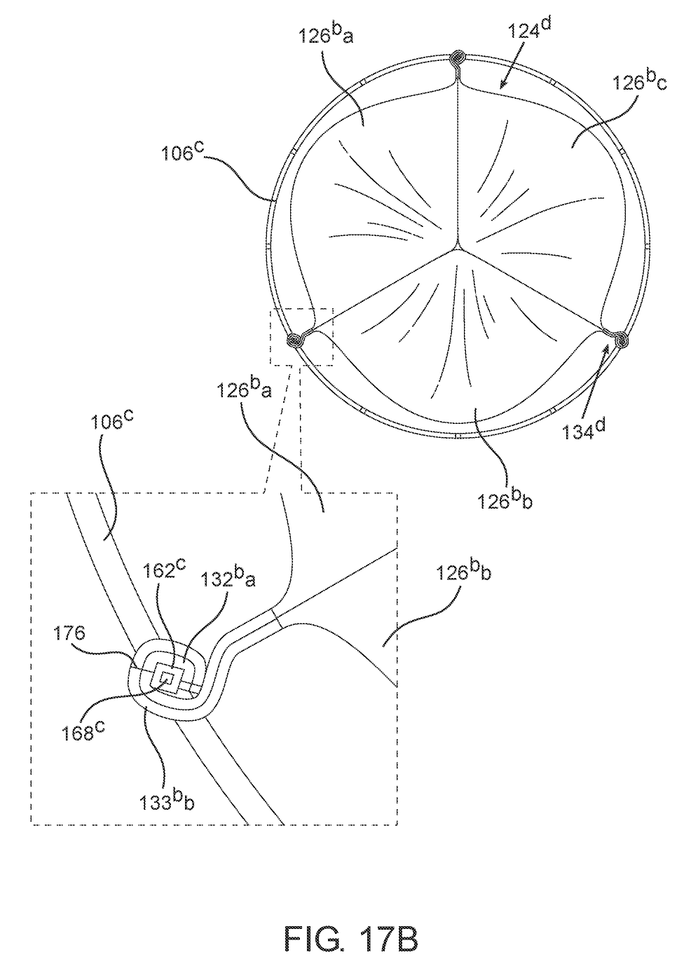

FIGS. 17A and 17B show a top views of a prosthetic valve 100^{c} having a leaflet assembly 124^{d} mounted within its frame 106^{c}, prior to and after leaflets adjustment, as shown in FIGS. 15A and 15B, respectively. The difference is that leaflets assembly 124^{d} comprises asymmetrical leaflets 126^{b}, wherein the different lengths between their short tabs 132^{b} and long tabs 133^{b} are chosen such that both leaflet tabs will terminate substantially at the same termination points, when wrapped over a single adjustable arm 162^{c}, as shown in the zoomed-in region of FIGS. 17A-17B.

According to some embodiments, there is provided a method for assembling a commissure assembly (134), comprising steps of: (1) wrapping a tab (132a) of one leaflet (126a) over an adjustable arm 162^{c}; (2) wrapping another tab of an adjacent leaflet (126b) over the previous tab; and (3) suturing both tabs to the adjustable arm 162^{c} via a suture 176 extending through both tabs and apertures 164 of the adjustable arm 162^{c}. In some embodiments, the step of suturing includes passing the suture 176 through portions of the tabs disposed over opposite sides of the adjustable arm 162^{c}.

According to some embodiments, the step of wrapping a tab of one leaflet over the adjustable arm includes wrapping a short tab (132^{b}a) of the one leaflet (126^{b}a) over the adjustable arm 162^{c}, and the step of wrapping another tab includes wrapping a long tab (132^{b}b) of the adjacent leaflet (126^{b}a) over the short tab (132^{b}a).

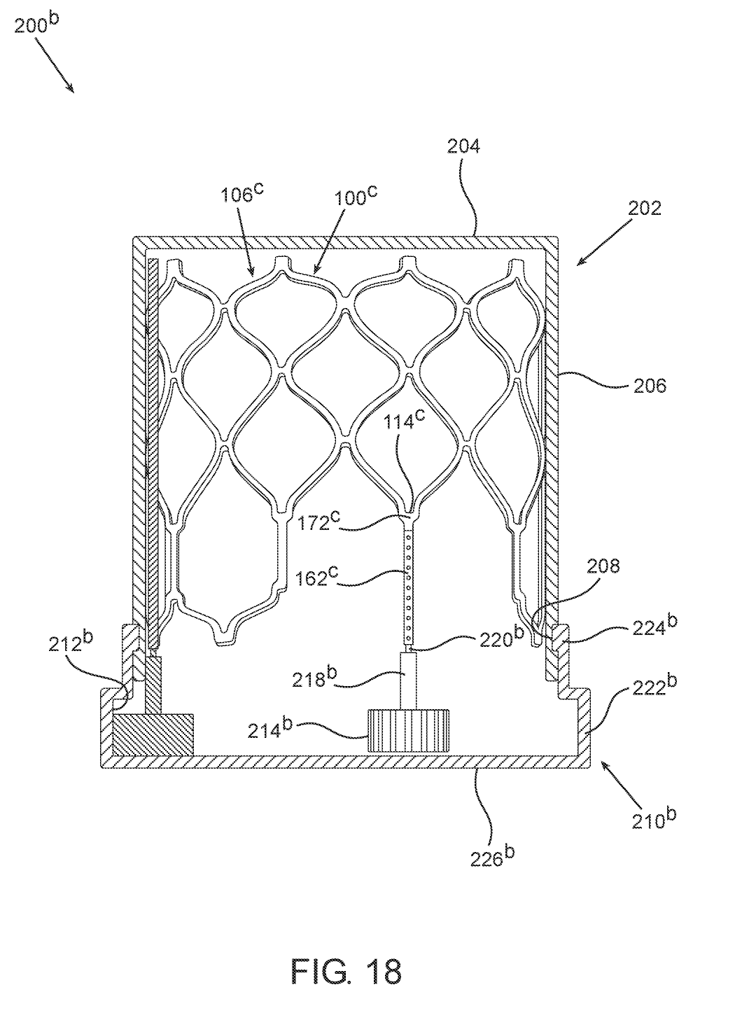

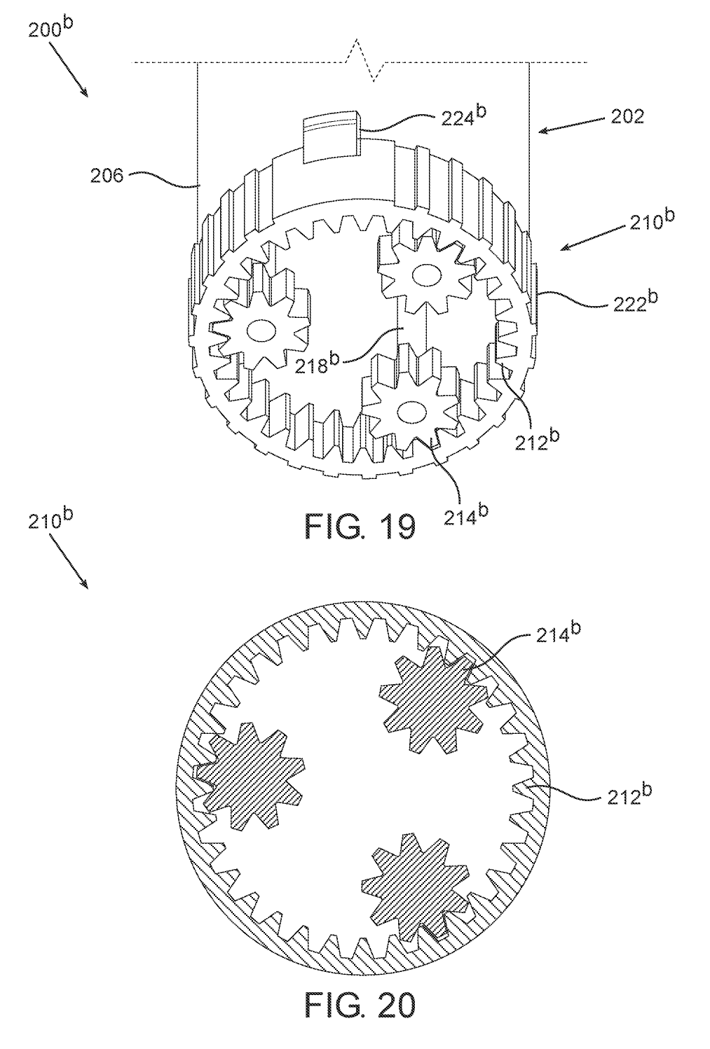

FIGS. 18-20 show views of an exemplary commissure adjustment assembly 200^{b}, similar to the views shown in FIGS. 11-13 shown for the commissure adjustment assembly 200^{a}. The commissure adjustment assembly 200^{b} is similar to the commissure adjustment assembly 200^{a} in all respects, except that it is adapted for use with a prosthetic valve 100^{c} equipped with adjustable commissure support 160^{c} that have a single adjustable arms 162^{c} extending from each support base 172^{c}.

The commissure adjustment assembly 200^{b} includes the same valve holder 202 described hereinabove with respect to FIGS. 11-13. The adjustment handle 210^{b} is similar to adjustment handle 210^{a} described hereinabove with respect to FIGS. 11-13, except that it does not include secondary pinion gears (216). As shown, adjustment handle 210^{b} includes only primary pinion gears 214^{b}, meshed with the internal teeth of the driver gear 212^{b}.

The driving rod 218^{b} extending from each primary pinion gear 214^{b} is configured to engage, via its driving head 220^{b}, with the single adjustable arm 162^{c} of an adjustable commissure support 160^{c}. Thus, when each primary pinion gears 214^{b} are rotated by the driver gear 212^{b}, they rotate the engagement portions 166^{c} of the adjustable arms 162^{c} therewith, which in turn facilitate twisting or rotation of the adjustable arms 162^{c}.

According to some embodiments, there is provided a method for adjusting the tension of leaflets (126) of a prosthetic valve (100), comprising steps of: (1) placing a prosthetic valve (100) having a plurality of adjustable commissure supports (160) within a valve holder (202) of a commissure adjustment assembly (200); (2) releasably coupling an adjustment handle (210) to the valve holder (202), such that driving heads (220) of driving rods (218) extending from pinion gears, that are positioned within a driver gear, engage with engagement portions (166) of adjustable arms (162) of the adjustable commissure supports (160); and (3) rotating the driver gear (212) to facilitate rotation of the pinion gears rotatable thereby, which in turn rotate the engagement portions therewith.

According to some embodiments, the step of placing the prosthetic valve (100) within the valve holder (202), includes a step of expanding the prosthetic valve (100) against the annular holder body (206) of the valve holder (202).

According to some embodiments, each adjustable commissure support 160^{c} includes a single adjustable arm 162^{c}, and the pinion gears include primary pinion gears 214^{b} meshed with the driver gear 212^{b}. In such embodiments, the step of coupling includes engaging the driving heads 220^{b} of driving rods 218^{b} extending from the primary pinion gears 214^{b} with the engagement portions 166^{c} of the single adjustable arms 162^{c} of the adjustable commissure supports 160^{c}. Furthermore, in such embodiments, the step of rotating includes rotating the driver gear 212^{b} to facilitate rotation of the primary pinion gears 214^{b} meshed therewith.

According to some embodiments, each adjustable commissure support (160^{a}, 160^{b}) includes two adjustable arms (162^{b}, 162^{c}), and the pinion gears include primary pinion gears 214^{a} meshed with the driver gear 212^{a}, and secondary pinion gears 216^{a} meshed with the primary pinion gears 214^{a} but not with the driver gear 212^{a}. In such embodiments, the step of coupling includes engaging the driving head 220^{a} of the driving rod 218^{a} extending from each primary pinion gear 214^{a} with one adjustable arm (162^{b}a, 162^{c}a) of an adjustable commissure support (160^{a}, 160^{b}), and engaging the driving head 220^{a} of the driving rod 218^{a} extending from the secondary pinion gear 216^{a} meshed with the with the primary pinion gear 214^{a}, with the other adjustable arm (162^{b}b, 162^{c}b) of the same adjustable commissure support (160^{a}, 160^{b}). Furthermore, in such embodiments, the step of rotating includes rotating the driver gear 212^{a} to facilitate rotation of the primary pinion gears 214^{a} and the secondary pinion gears 216^{a} in opposite rotational directions.

In some implementations of the current invention, adjustment of the leaflet assembly (124) mounted within the prosthetic valve (100) is performed during the implantation procedure. For example, adjustment of the leaflet assembly (124) can be facilitated by a delivery apparatus (12) coupled to the prosthetic valve (100) during the implantation procedure.

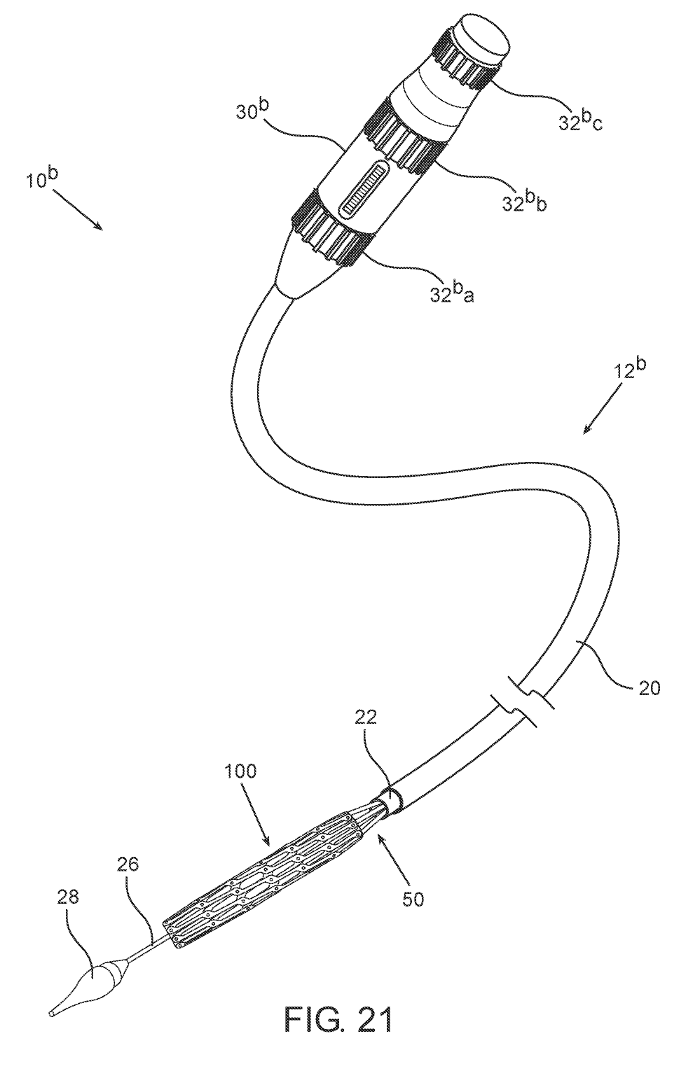

FIG. 21 shows a view in perspective of another type of a delivery assembly 10^{b} which can be used for delivering and implanting a prosthetic valve (100) carried thereby, as well as adjusting the tension of leaflets (126) mounted within the prosthetic valve (100). The delivery assembly 10^{b} includes a delivery apparatus 12^{b} that can be similar to the delivery apparatus 12^{a} described hereinabove with respect to FIG. 1, except that it may further include a delivery shaft 22 extending over the nosecone shaft 26 and optionally over the balloon catheter 24, and a plurality of adjustment assemblies 50 extending from the handle 30^{b} through the delivery shaft 22, and are releasably coupled to the adjustable commissure supports (160) of the prosthetic valve (100).

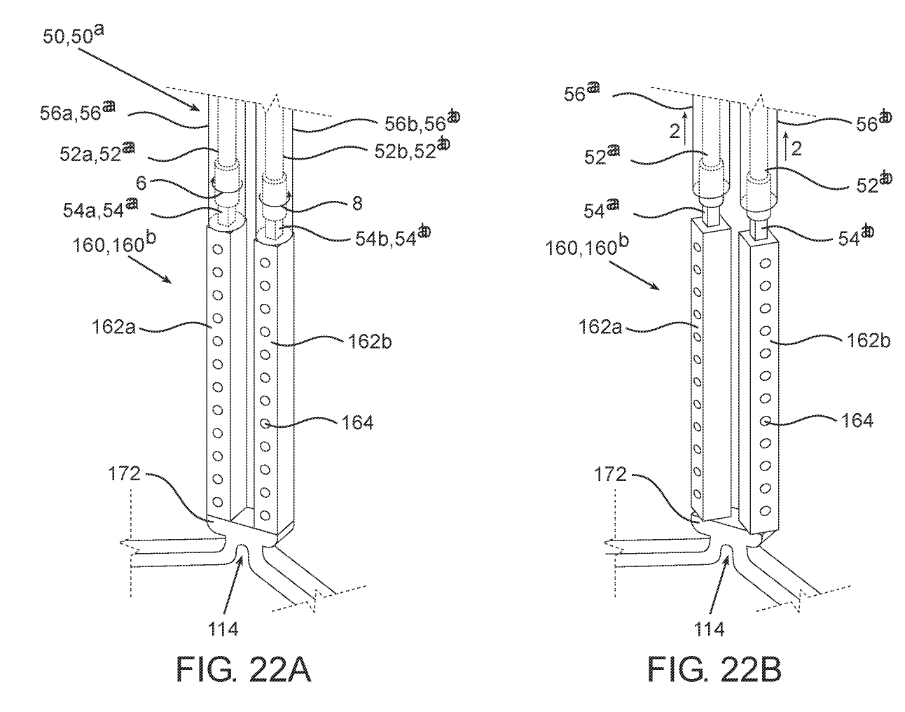

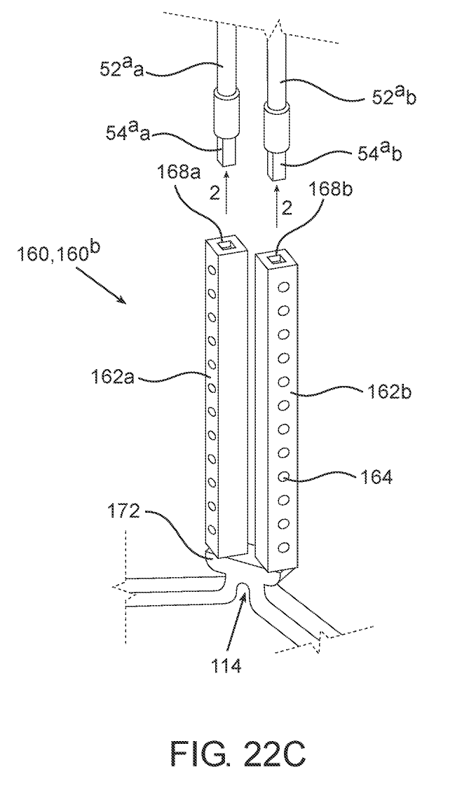

FIGS. 22A-22C show steps of manipulating an adjustable commissure support 160 via an adjustment assembly 50 of a delivery apparatus 12^{b}, according to some applications of the current invention. Each adjustment assembly 50 extends from the handle 30^{b} through the delivery shaft 22, and includes at least one adjustment arm 52 equipped with a driving head 54 at its distal end, the driving head 54 releasably coupled to the engagement portion 166 of the adjustable arm 162. The adjustment assembly 50 can optionally include an adjustment sleeve 56 extending from the handle 30^{b} over the adjustment arm 52. The adjustment arm 52 and the adjustment sleeve 56 can be movable longitudinally relative to each other in a telescoping manner.

The driving heads 54 are shaped with complementary shapes to those of the engagement portions 166. For example, if the engagement portion 166 of each adjustable arm 162 comprises a socket 168, the driving head 54 is shaped as a non-cylindrical extension configured to be inserted into the socket 168, such that rotation of the driving head 54 will rotate the engagement portion 166 therewith. More specifically, if the socket 168 is shaped as a rectangular socket, as shown in FIGS. 6-10B, for example, the driving head 54 will can be shaped as a rectangular extension, dimensioned for insertion thereof into the respective socket 168.

The driving heads 54 are releasably engaged with the engagement portions 166 of the adjustable arms. As shown in FIG. 22C, for example, the driving heads 54 can be shaped as distally oriented extension, having a shape which is complementary to that of the sockets 168 (which can be a rectangular shape as shown in FIG. 22C). The adjustment arms 52 are torque transmitting arms, configured to transmit torque from the handle 32^{b} to the engagement portions 166. The adjustment arms 52 can be, for example, wires, cables, rods, or tubes.

The adjustment sleeve 56 can be coaxially disposed over the adjustment arms 52, including the driving head 54, and may serve to prevent spontaneous disengagement between the driving head 54 and the engagement portion 166. The adjustment sleeve 56 may be in contact with the proximal end of the adjustable arm 162, for example - having the distal end of the adjustment sleeve 56 pressed against the proximal end of the engagement portions 166, as shown if FIG. 22A.

According to some embodiments, the adjustment assembly 50^{a} comprises two adjustment arms 52^{a}, configured to engage with two adjustable arms 162 of an adjustable commissure support 160, as shown in FIGS. 22A-22C. The adjustment assembly 50^{a} can optionally include two adjustment sleeves 56^{a}, each disposed over one of the adjustment arms 52^{a}.

A prosthetic valve 100 can be delivered in a crimped state by the delivery apparatus 12^{b} through the patient’s vasculature, and expended against the native anatomy when reaching the desired implantations site. The implantation procedure can be visualized via various imaging modalities, such as fluoroscopy, ultrasound and the like, that may provide an indication of the prosthetic valve’s expanded diameter once expansion is completed. At this stage, the adjustment assemblies 50 can be utilized to adjust the tension of the leaflet (126) mounted within the expanded valve, so as to allow proper coaptation thereof at the actual expanded diameter of the frame (106).

As shown in FIG. 22A, one adjustment arm 52^{a}a is coupled to one adjustable arm 162a, by having its driving head 54^{a}a engaged with the engagement portion 166a, for example, inserted into the socket 168a. The other adjustment arm 52^{a}b is coupled to the adjacent adjustable arm 162b, by having its driving head 54^{a}b engaged with the engagement portion 166b, for example, inserted into the socket 168b. Rotation of the adjustment arms 52^{a} rotates the engagement portions 166 therewith.

In the exemplary illustration of FIG. 22A, both adjustment arms 52^{a} are rotated in opposite rotation directions. For example, the adjustment arm 52^{a}a is rotated in first rotational direction 6 (e.g., clockwise), while the adjacent adjustment arm 52^{a}b is rotated in an opposite, second rotational direction 8 (e.g., counterclockwise), to rotate the engagement portion 166a and 166b of the adjustable arms 162a and 162b in the same directions. This opposite directions of rotation applied to the adjustment arm 52 are desirable in case the tabs (132) are wrapped around the adjustable arms 162 in opposite direction, which will result in the tabs being wound around the respective adjustable arms 162 in opposite directions as well, similar to the configuration described hereinabove with respect to FIGS. 7A-7B, during rotational movement of the adjustment arms 52^{a}.

It is to be understood that in alternative configurations, wherein both tabs (132) are wrapped around the respective adjustable arms (162) in the same direction, the adjustment arms 52^{a}a and 52^{a}b will be rotated in the same rotational direction.

FIGS. 22A-22C illustrate the adjustment assembly 50^{a} used in combination with an adjustable commissure support 160^{a} having both of the adjustable arms 162^{a} rotatably coupled to the support base 172^{a}, such that once the engagement portions 166^{a}a and 166^{a}b are rotated, the adjustable arms 162^{a}a and 162^{a}b are rotated therewith, relative to the support base 172^{a}. It is to be understood that the adjustment assembly 50^{a} can be used in combination with an adjustable commissure support 160^{b}, having both of the adjustable arms 162^{b} rigidly attached to the support base 172^{b}, in the same manner, mutatis mutandis. In this case, similar rotational movement of the engagement portions 166^{a}a and 166^{a}b, resulting from the rotation of the actuation arms 52^{a}a and 52^{a}b, will serve to twist the adjustable arms 162^{a}a and 162^{a}b between their engagement portions 166^{a} and the support base 172^{a}, similar to the configuration of FIGS. 9A-9B.

Once the adjustable arms 162 are rotated or twisted to the desired position, so as to provide the sufficient tension to the leaflets (126) at the actual expanded diameter of the frame (106), the adjustment assemblies 50^{a} can disengage from the adjustable commissure supports 160, to allow retrieval of the delivery apparatus 12^{b}.

According to some embodiments, disengagement of the adjustment assemblies 50^{a} includes first pulling the adjustment sleeves 56^{a} in a proximally oriented direction 2, away from the adjustable arms 162, as shown in FIG. 22B, and then pulling the adjustment arms 52^{a} in the same proximal direction 2 as well, thereby disengaging the driving heads 54^{a} from the engagement portion 166 (for example, pulling the driving heads 54^{a} out of the sockets 168), as shown in FIG. 22C. Alternatively, both the adjustment sleeves 56^{a} and the adjustment arms 52^{a} can be simultaneously pulled together in the proximal direction 2. In yet further alternative embodiments, in which the adjustment arms 52^{a} do not include adjustment sleeves (56), disengagement of the adjustment assemblies 50^{a} includes only the pull of the driving heads 54^{a} in a proximal direction 2, as shown in FIG. 22C.

Once the adjustment assemblies 50^{a} are disengaged, they can be further pulled away and retracted, with the rest of the delivery apparatus 12^{b}, from the patient’s body, leaving the prosthetic valve (100) implanted in the patient.

While FIGS. 22A-22C show an adjustment assembly 50^{a} equipped with two adjustment arms 52^{a}, used in combination with adjustable commissure supports 160 that have two adjustable arms 162 extending from the support base 172, other embodiments of the adjustment assembly 50 include a single adjustment arm 52 releasably coupled to a single adjustable arm 162^{c} of an adjustable commissure supports 160^{c}, such as shown in FIG. 14. The adjustment assembly 50 equipped with a single adjustment arm 52 can be utilized in the same manner described hereinabove with respect to FIGS. 22A-22C, mutatis mutandis.

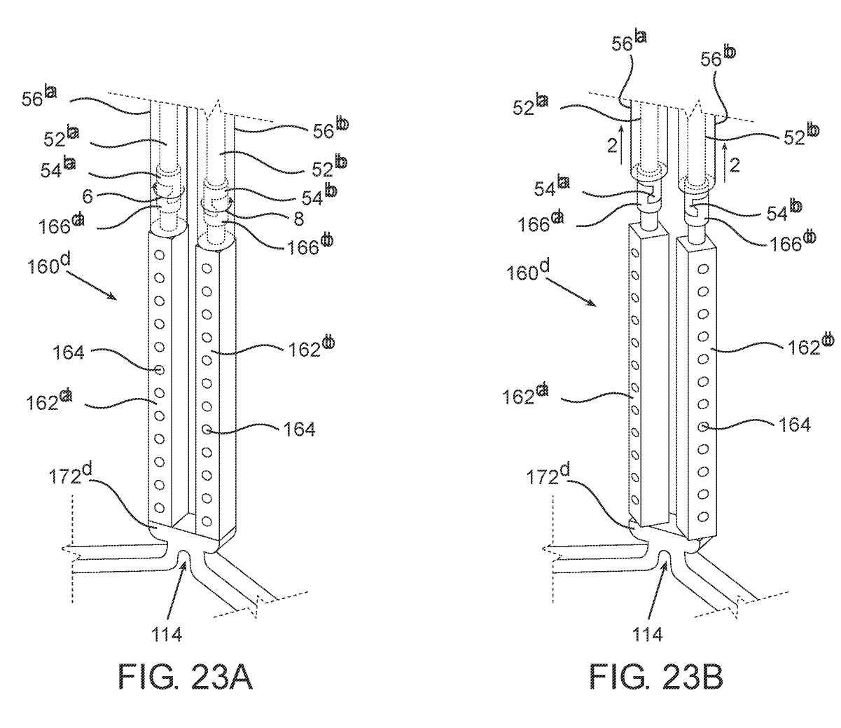

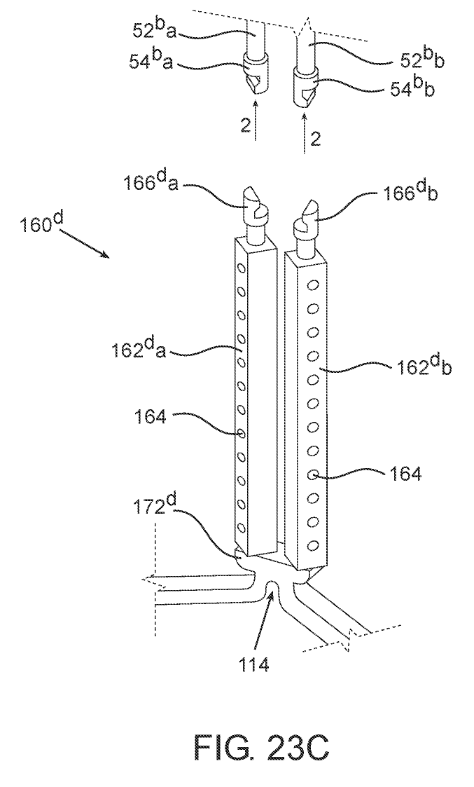

FIGS. 23A-23C show steps of manipulating adjustable commissure supports 160 via adjustment assemblies 50, which are similar to those shown in FIGS. 22A-22C, except for some modifications relating to the type of engagement there-between. Specifically, FIGS. 23A-23C show an adjustable commissure support 160^{d}, which can be similar to any of the embodiments described hereinabove with respect to other types of adjustable commissure supports 160^{a}, 160^{b}, 160^{c}, except that the engagement portion 166^{d} does not include a socket (168), but is rather formed as a proximally oriented extension having formed as a drive-screw coupler. The adjustment assembly 50^{b} shown in FIGS. 23A-23C is similar to any of the embodiments described hereinabove with respect to adjustment assembly 50^{a}, except that the driving head 54^{b} is not configured to be inserted into a socket, but is rather formed as a drive-screw coupler having a shaped complementary to that of the engagement portion 166^{d}.

The driving head 54^{b} is configured to engage with the complementary-shaped engagement portion 166^{d} in a “hand-shake” type of engagement, as shown in FIG. 23A, such that rotational movement of the adjustment arm 52^{d} will force the engagement portion 166^{d} of the adjustable arms 162^{d} to rotate therewith. The adjustment sleeve 56^{d} covering both the driving head 54^{b} and the engagement portion 166^{d}, as shown in FIG. 23A, serves to prevent undesirable disengagement therebetween, as the natural lateral/radial forces acting on the driving head 54^{b} and the engagement portion 166^{d} strive to force them away from each other during such rotational movements.

Once rotation of the adjustment arms 52^{b} (FIG. 23A) is completed, the adjustment sleeves 56^{b} are pulled proximally 2, as shown in FIG. 23B, so as to expose the driving heads 54^{b} and the engagement portion 166^{d}, which will allow them to disengage from each other. As shown in FIG. 23C, the drive-screw coupler shapes of both the driving head 54^{b} and the engagement portion 166^{d}, include complementary angled surfaces, which are angled with respect to their longitudinal axis, allowing the driving head 54^{b} and the engagement portion 166^{d} to slide over and away from each other when pulled away from each other.

FIGS. 23A-23C illustrate the adjustable commissure support 160^{d} with two adjustable arms 162^{d} that are rotatably coupled to the support base 172^{d}, similar to the configuration of adjustable commissure support 160^{b}. It is to be understood that the features of adjustable commissure support 160^{d} can be similarly implemented in combination with adjustable commissure support 160^{b} provided with twistable adjustable arms 162^{b}, such that each adjustable arms 162^{b}, which is configured to twist between its engagement portion 166^{b} and the support base 172^{b}, includes an engagement portion formed as a drive-screw coupler, such as illustrated for engagement portion 166^{d} in FIGS. 23A-23C, configured to engage in a “hand-shake” type of engagement with a complementary shaped driving head 54^{b}.

While FIGS. 23A-23C show an adjustment assembly 50^{b} equipped with two adjustment arms 52^{b}, used in combination with adjustable commissure supports 160 that have two adjustable arms 162 extending from the support base 172, other embodiments of the adjustment assembly 50 include a single adjustment arm 52 releasably coupled to a single adjustable arm 162^{c} of an adjustable commissure supports 160^{c}, such as shown in FIG. 14. It is to be understood that the features of adjustable commissure support 160^{d} can be similarly implemented in combination with adjustable commissure support 160^{c} provided with a single adjustable arm 162^{c}, such that the adjustable arm 162^{c} includes an engagement portion formed as a drive-screw coupler, such as illustrated for engagement portion 166^{d} in FIGS. 23A-23C. The adjustment assembly 50 equipped with a single adjustment arm 52 having a screw-drive shaped driving head 54^{d}, can be utilized in the same manner described hereinabove with respect to FIGS. 23A-23C, mutatis mutandis.

According to some embodiments, there is provided a method for adjusting the tension of leaflets (126) of a prosthetic valve (100), comprising steps of: (1) expanding the prosthetic valve (100) that includes a plurality of adjustable commissure supports, to a final expanded diameter; (2) rotating adjustment arms (52) that extend from a handle (30^{b}) of a delivery apparatus (12^{b}), and are coupled via driving heads (54) thereof to engagement portions (166) of adjustable arms(162) of the adjustable commissure supports (160), to facilitate rotation of the engagement portions (166) therewith; and (3) disengaging the driving heads (54) from the engagement portions (166).

According to some embodiments, the step of disengaging the driving heads includes pulling the driving heads (54) proximally away from the engagement portions (166).

According to some embodiments, the step of disengaging the driving heads includes pulling adjustment sleeves (56) that are disposed over the adjustment arms (52) in a proximal direction 2, so as to expose the driving heads (54).

According to some embodiments, the step of disengaging the driving heads includes simultaneously pulling the adjustment arms (52) with adjustment sleeves (56) disposed thereover.

According to some embodiments, the step of rotating the adjustment arms includes rotating each couple of adjustment arms (52) engaged with two adjustable arms (162) that extend from a single support base (172), in opposite rotational directions.

It is to be understood that the series of steps disclosed hereinabove can be performed either in a patient’s body, during the process of prosthetic valve implantation, or prior to an actual implantation procedure, for example to test the performance of the equipment. Utilization of the method can be performed, for example, in an experimental setup, to test the amount of rotation required for each frame (106) expansion diameter, so as to achieve proper leaflets (126) coaptation.

When utilized during an implantation procedure, within a patient’s body, the method can further comprise a step of retrieving the delivery apparatus (12^{b}) from the patient’s body.

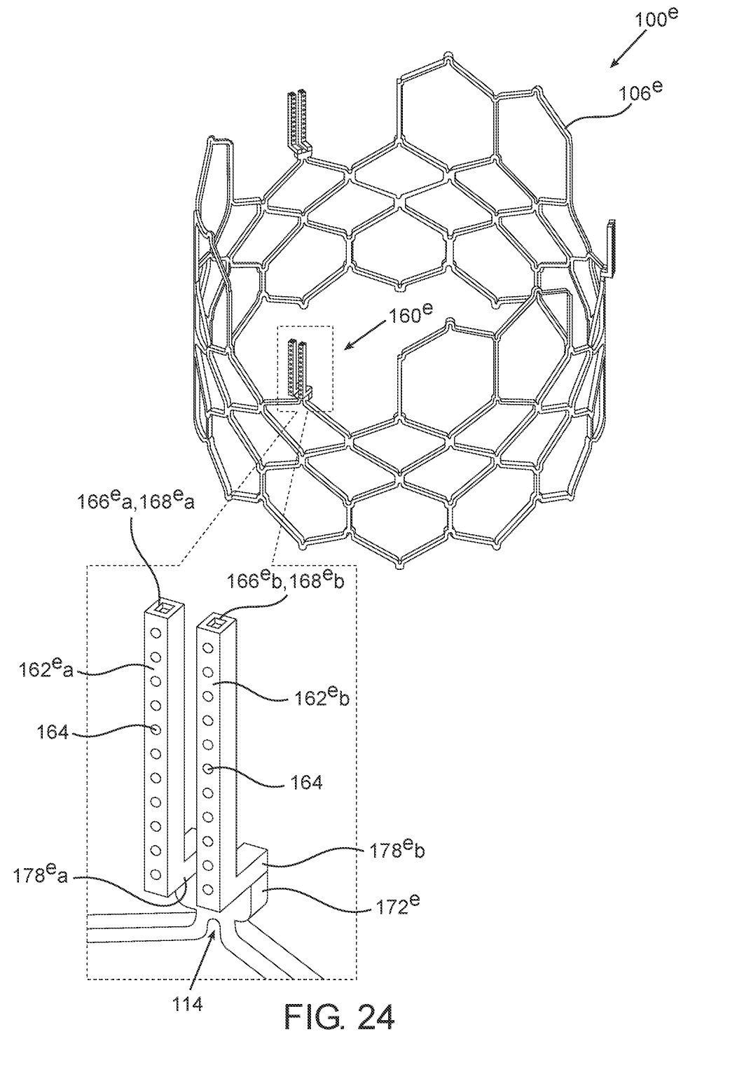

FIG. 24 shows another type of a prosthetic valve 100^{e} provided with adjustable commissure supports 160^{e}, according to some applications of the current invention. An adjustable commissure supports 160^{e} can be similar to any of the embodiments described hereinabove with respect to other types of adjustable commissure supports 160^{a}, 160^{b}, 160^{c}, and/or 160^{d}, except that the support base 172^{e} further includes offsetting extensions 178^{e}, extending radially outward, thereby offsetting the adjustable arms 162^{e}, that extend therefrom, radially away from the frame 106^{e}.

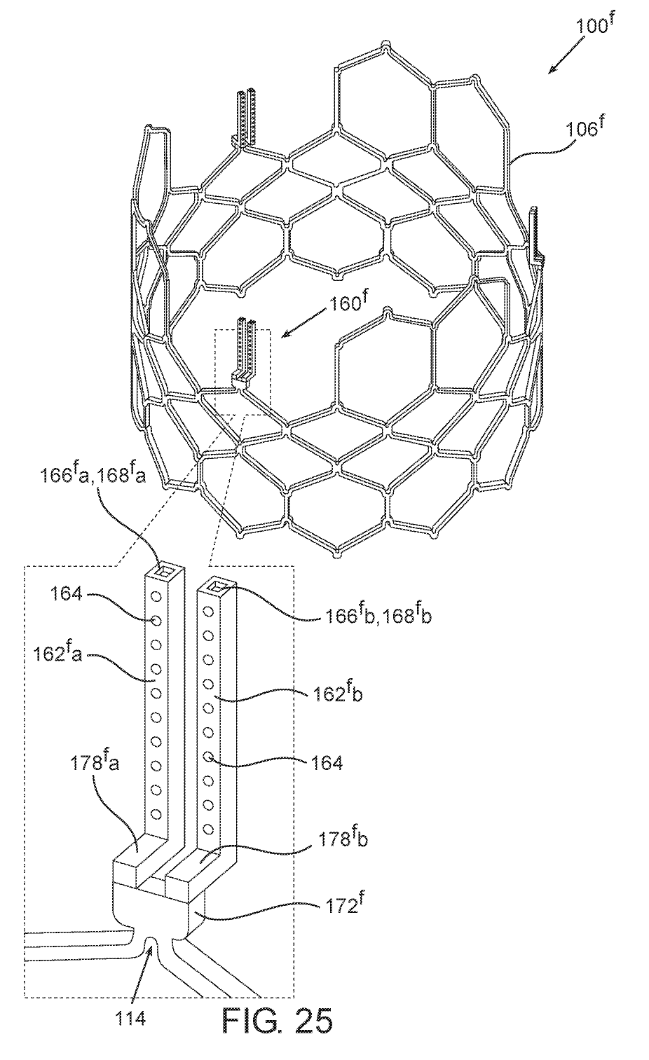

FIG. 25 shows another type of a prosthetic valve 100^{f} provided with adjustable commissure supports 160^{f}, according to some applications of the current invention. An adjustable commissure supports 160^{f} can be identical to the adjustable commissure supports 160^{e}, except that the offsetting extensions 178^{f} are extending radially inward, thereby offsetting the adjustable arms 162^{f}, that extend therefrom, radially inward with respect to the frame 106^{e}.

In some embodiments, the offsetting extensions 178^{e}, 178^{f} can be integrally formed with the support base 172^{e}, 172^{f}. In some embodiments, the offsetting extensions 178^{e}, 178^{f} can be integrally formed with the adjustable arms 162^{e}, 162^{f}.

It is to be understood that features of adjustable commissure supports 160^{e}, 160^{f} which include offsetting extensions 178^{e}, 178^{f}, can be combined with any other features of any other type of adjustable commissure supports 160^{a}, 160^{b}, 160^{c}, and/or 160^{d} described hereinabove. For example, each of the adjustable commissure supports 160^{e}, 160^{f} is illustrated with two offsetting extensions, such as offsetting extension 178^{e}a from which adjustable arm 162^{e}a extends, and offsetting extension 178^{e}b from which adjustable arm 162^{e}b extends, or such as offsetting extension 178^{f}a from which adjustable arm 162^{f}a extends, and offsetting extension 178^{f}b from which adjustable arm 162^{f}b extends, each adjustable commissure supports 160^{e}, 160^{f} can also include a single offsetting extension 178^{e}, 178^{f}, from which a single adjustable arm 162, such as the single adjustable arm 162^{c} illustrated in FIG. 14, extends.

The adjustable arms 162^{e}, 162^{f} can be either rigidly attached to the offsetting extensions 178^{e}, 178^{f}, to enable twisting thereof, such as illustrated for adjustable arms 162^{a} in FIGS. 9A-9B, or rotatably coupled to the offsetting extensions 178^{e}, 178^{f} in a similar manner to that illustrated for adjustable arms 162^{b} in FIGS. 10A-10B, in which case the base threaded portions (174) are comprised in the offsetting extensions 178^{e}, 178^{f}.

The engagement portions 166^{e}, 166^{f} can comprise sockets 168^{e}, 168^{f}, as illustrated in FIGS. 24-25, or may be alternatively formed as drive-screw couplers, similar to engagement portion 166^{d} illustrated in FIGS. 23A-C.

Prosthetic valves 100^{e}, 100^{f} equipped with adjustable commissure supports 160^{e}, 160^{f} can be used in combination with any type of a commissure adjustment assembly 200, in the same manner described hereinabove, or in combination with any type of a delivery apparatus 12^{b} equipped with adjustment assemblies 50, in the same manner describes hereinabove, mutatis mutandis.

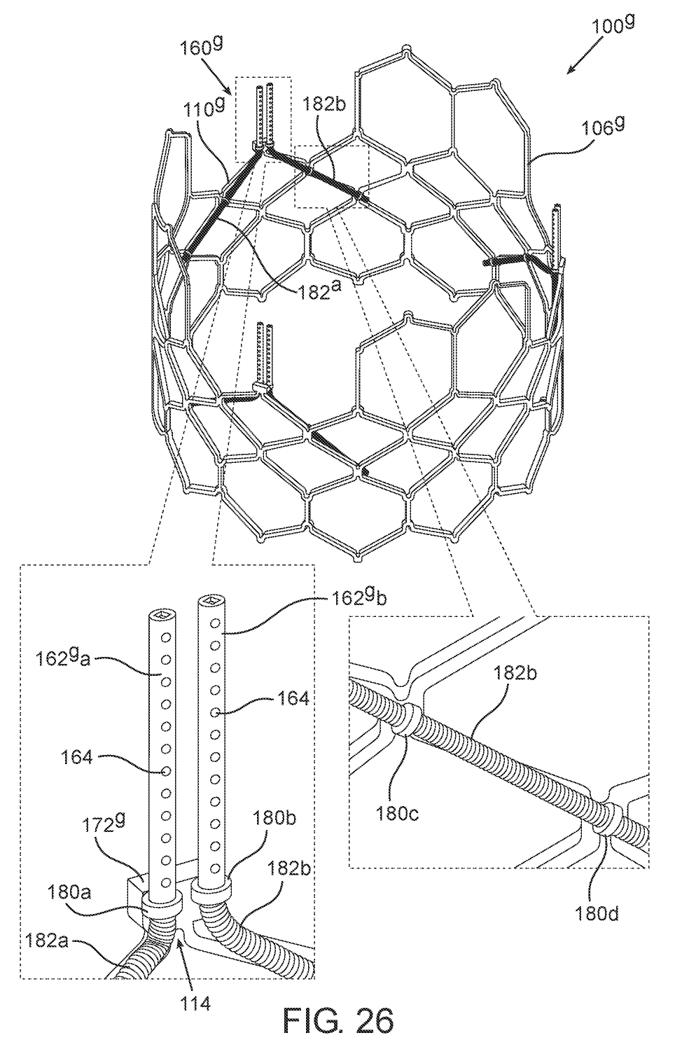

FIG. 26 shows another type of a prosthetic valve 100^{g} provided with adjustable commissure supports 160^{g}, according to some applications of the current invention. The commissure support 160^{g} differs from other types of commissure supports 160^{a}, 160^{b}, 160^{g}, 160^{d}, 160^{e} and 160^{f}, described hereinabove, in that the commissure support 160^{g} is configured to adjust the tension of the leaflets 126 not only winding or unwinding the tabs 132 around adjustable arms 162^{g}, but also by winding or unwinding at least a portion of the cusp edges 128 of the leaflets 126, around scallop adjustment arms 182 thereof.

An adjustable commissure support 160^{g} comprises two adjustable arms 162^{g} which are rotatably coupled to a support base 172^{g}. The support base 172^{g} can include two eyelets 180 configured to rotatably couple with the adjustable arms 162^{g}. The support base can be either integrally formed with the frame 106^{g}, such as being integrally formed with a junction 114 thereof, meaning that the junction 114 can be formed, during the manufacturing process of the frame 106^{g}, to be shaped as, and to serve as, the support base 172^{g}, as illustrated in FIG. 26. Alternatively, the support base 172^{g} can be provided as a separate component, attached to a junction 114 of the prosthetic valve 100^{g}.

The adjustable commissure support 160^{g} further comprises scallop adjustment arms 182, wherein each scallop adjustment arm 182 extends continuously from a respective adjustable arm 162^{g}, and may be coupled to the frame 106^{g} via additional eyelets 180 extending from junctions 114 or struts 110 thereof.

According to some embodiments, each adjustable arm 162^{g} and scallop adjustment arm 182 are integrally formed, meaning that they may be manufactured and provided as a single continuous component. According to other embodiments, the adjustable arm 162^{g} and the scallop adjustment arm 182 are provided as two separate components which are rigidly attached to each other, for example - at the region of the support base 172^{g}, such that when the adjustable arm 162^{g} is rotated, the scallop adjustment arm 182 rotates therewith. According to some embodiments, the scallop adjustment arm 182 comprises a torque transmitting shaft.

According to some embodiments, the eyelets 180 are eyelets through which the adjustable arms 162^{g} and/or the scallop adjustment arms 182 extend. According to some embodiments, the adjustable arms 162^{g} are threadedly engaged with the eyelets 180. According to some embodiments, the scallop adjustment arms 182 are threadedly engaged with the eyelets 180. According to some embodiments, at least some of the eyelets 180 include internal threads, and the adjustable arms 162^{g} and/or the scallop adjustment arm 182 include external threads at the region extending through threaded eyelets 180.

As shown in the zoomed-in regions of FIG. 26, a support base 172^{g} can include two eyelet-shaped couplers 180a and 180b, such that the adjustable arm 162^{g}a and/or the scallop adjustment arm 182a, extends through eyelet 180a, and such that the adjustment arm 162^{b} and/or the scallop adjustment arm 182b, extends through eyelet 180b. The adjustable arms 162^{g}a and 162^{g}b extend proximally from the eyelets 180a and 180b of the support base 172^{g}. The scallop adjustment arms 182a and 182b extend distally from the eyelets 180a and 180b of the support base 172^{g}, wherein each scallop adjustment arm 182 is coupled to the frame 106^{g} by passing through eyelets 180 extending from junction 114 or struts 110 thereof. For example, the scallop adjustment arm 182b is shown in the zoomed-in region of FIG. 26 to extend through eyelets 180c and 180d.

The eyelets 180 extend radially inward from the support bases 172^{g}, junctions 114 and/or struts 110, such that the adjustable arms 162^{g} and the scallop adjustment arms 182 are positioned radially inward with respect to the frame 106^{g}.

The adjustable arms 162^{g} comprise engagement portions 166^{g} that can be implemented according to any of the embodiments described for engagement portions 166 hereinabove, such as engagement portions 166^{g} that include non-cylindrical socket 168^{g}, or engagement portions 166^{g} that are shaped as drive-screw couplers, similar to engagement portion 166^{d} illustrated in FIGS. 23A-23C.

In some embodiments, the adjustable arms 162^{g} comprise a plurality of apertures 164, implemented in the same manner described hereinabove for apertures 164 comprised within any other type of the adjustable arms 162.

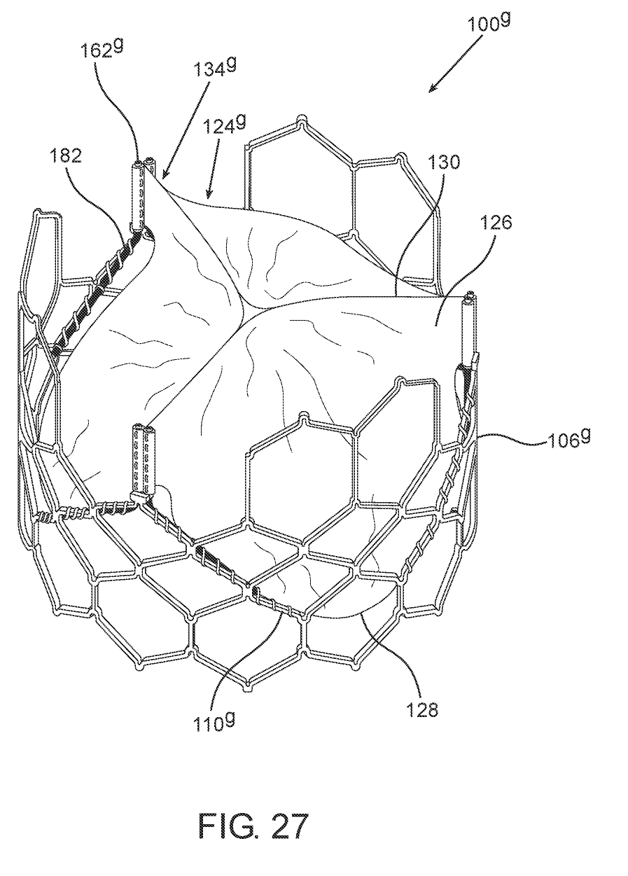

FIG. 27 shows a leaflet assembly 124^{g} mounted within the frame 106^{g} shown in FIG. 26. The tabs 132 of each couple of adjacent leaflets 126 within the same commissure assembly 134^{g} are attached to the adjustable arms 162^{g}, such that the tabs 132 are wrapped around the respective adjustable arms 162^{g} in opposite directions, similar to the configuration described hereinabove with respect to FIGS. 7A-7B. Portions of the cusp edges 128 of the leaflets 126 are attached to the scallop adjustment arms 182 along the scallop line of the leaflet assembly 124^{g}.

As shown, the scallop adjustment arms 182 are coupled to the frame 106^{g} in a manner that generally tracks the scallop line of the leaflet assembly 124^{g}. The cusp edge 128 of each leaflet 126 can be attached to scallop adjustment arms 182 of two adjustable commissure supports 160^{g} positioned on both sides of the leaflet 126.

In use, ₜₕe adjustable arms 162^{g} can be rotated in a similar manner to their rotation described throughout the current specification, wherein both adjustable arms 162^{g} of each adjustable commissure supports 160^{g} are necessarily rotated in opposite direction. It is to be noted that unlike other embodiments of other type of adjustable commissure supports 160, the adjustable arms 162^{g} are configured only to rotate and not to twist, each adjustable commissure support 160^{g} necessarily includes two adjustable arms 162^{g} and not only one, and each couple of adjustable arms 162^{g} of the same adjustable commissure support 160^{g} are necessarily rotated in opposite direction with respect to each other, and not in the same direction.

When the adjustable arms 162^{g} are rotated, the scallop adjustment arms 182 extending therefrom are rotated therewith, which in turn cause the portions of the cusp edges 128 attached thereto, to wrap around the respective scallop adjustment arms 182 in a similar manner. Thus, the adjustable commissure supports 160^{g} enable leaflet tension adjustment not only vit the tabs 132 of the leaflets 126, which will shorten or elongate the free edges 130, but also via the cusp edges 128, thereby providing a solution that enables the tension of the leaflets 126 to be adjusted in a manner that is more uniform along various regions of the leaflets 126.

The cusp edges 128 that are attached to the scallop adjustment arms 182 can be sutured thereto, for example by sutures 176. While not illustrated, the scallop adjustment arms 182 can include, in some embodiments, a plurality of apertures 164, similar to the apertures 164 extending through the adjustable arms 162, such that sutures 176 can extend through portions of the cusp edges 128 and such apertures 164 in a similar manner to that described for suturing tabs 132 to adjustable arms 162 hereinabove. In some configurations, it is preferable not to include eyelets 180 through which the scallop adjustment arms 182 can extend, along regions of scallop edge attachment to the scallop adjustment arms 182, in order to avoid interference with the attachment between the cusp edges 128 and the scallop adjustment arms 182. In such cases, the scallop adjustment arms 182 can extends through eyelets 180 extending from the support base 172^{g}, which may be positioned above (or proximal to) the region of attachment to the cusp edges 128, and another eyelet 180 extending inwardly from the frame 106^{g} below (or distal to) the region of attachment to the cusp edges 128.

It is to be understood that the adjustable arms 162^{g} of each adjustable commissure support 160^{g} are spaced from each other, forming a gap G′ therebetween, which can be dimensioned to allow tabs having a thickness T′ to extend therethrough, and optionally accommodate several layers of the tabs 132, in the same manner described hereinabove with respect to the gap G′ of FIG. 6.

In some implementations, the prosthetic valve 100^{g} can be used in combination with a commissure adjustment assembly 200^{a} that includes primary pinion gears 214^{a} and secondary pinion gears 216^{a}, in a similar manner to that described hereinabove with respect to FIGS. 11-13, mutatis mutandis. In some implementations, the prosthetic valve 100^{g} can be used in combination with any type of an adjustment assembly 50 as long as each adjustment assembly 50 includes two adjustment arms 52, and in a similar manner to that described hereinabove with respect to FIGS. 22A-23C, mutatis mutandis.

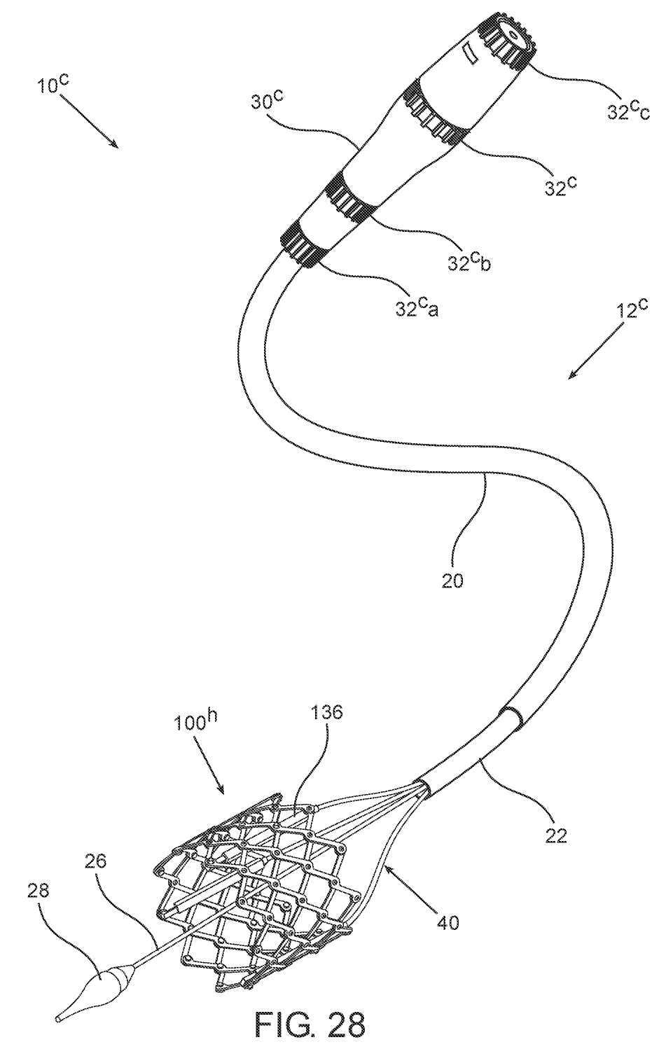

FIG. 28 shows a view in perspective of another type of a delivery assembly 10^{c} that can be used for delivering and implanting mechanically expandable prosthetic valve 100^{h}, according to some embodiments. The delivery apparatus 12^{c} of the delivery assembly 10^{c} can include a handle 30^{c} at a proximal end thereof, a nosecone shaft 26 extending distally from the handle 30^{c}, a nosecone 28 attached to the distal end of the nosecone shaft 26, a delivery shaft 22 extending over the nosecone shaft 26, and optionally an outer shaft 20 extending over the delivery shaft 22.

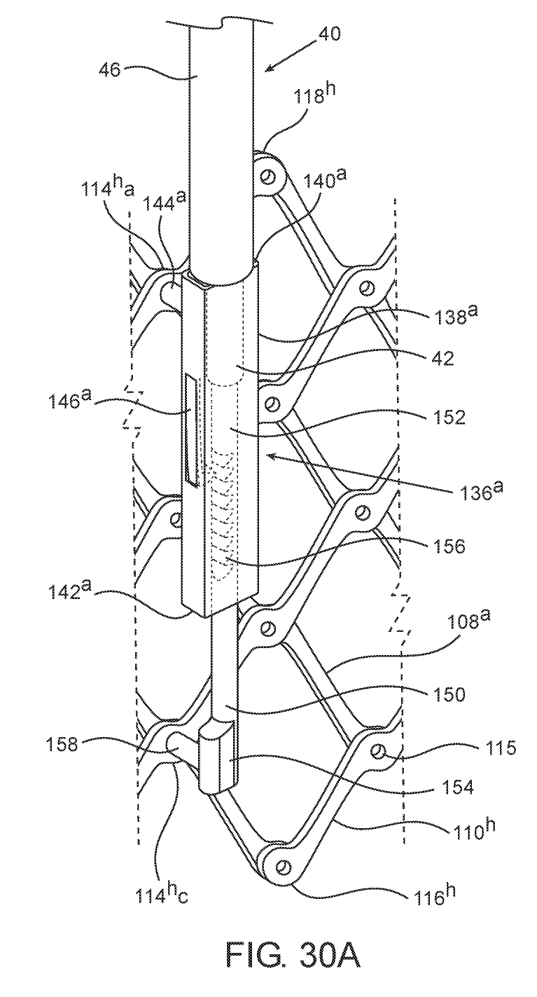

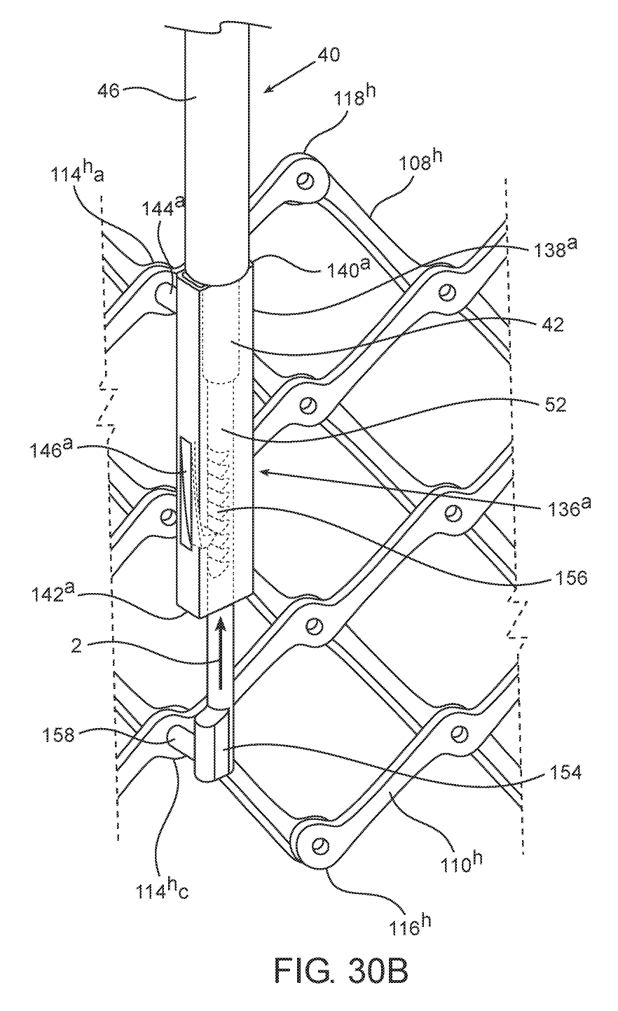

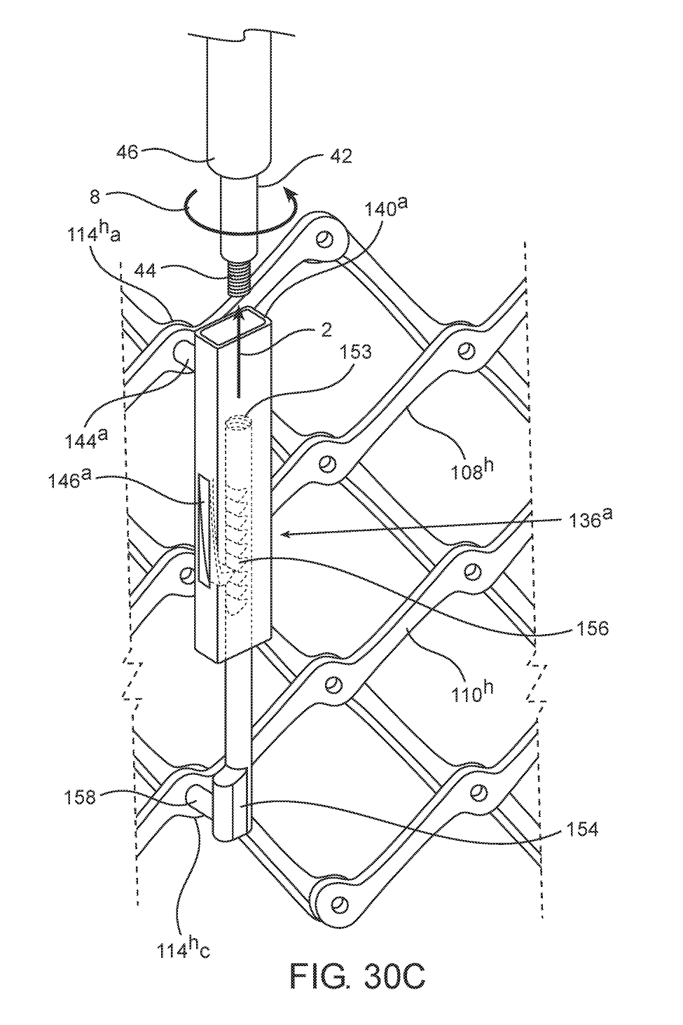

In the case of being used in combination with a mechanically expandable valve 100^{h}, the delivery apparatus 12^{c} can further comprise a plurality of actuation assemblies 40 extending from the handle 30^{c} through the delivery shaft 22. The actuation assemblies 40 can generally include actuation members 42 (hidden from view in FIG. 28, visible in FIGS. 30A-30C) releasably coupled at their distal ends to respective expansion and locking assemblies 136 of the prosthetic valve 100^{h}, and actuation support sleeves 46 disposed around the respective actuation members 42.

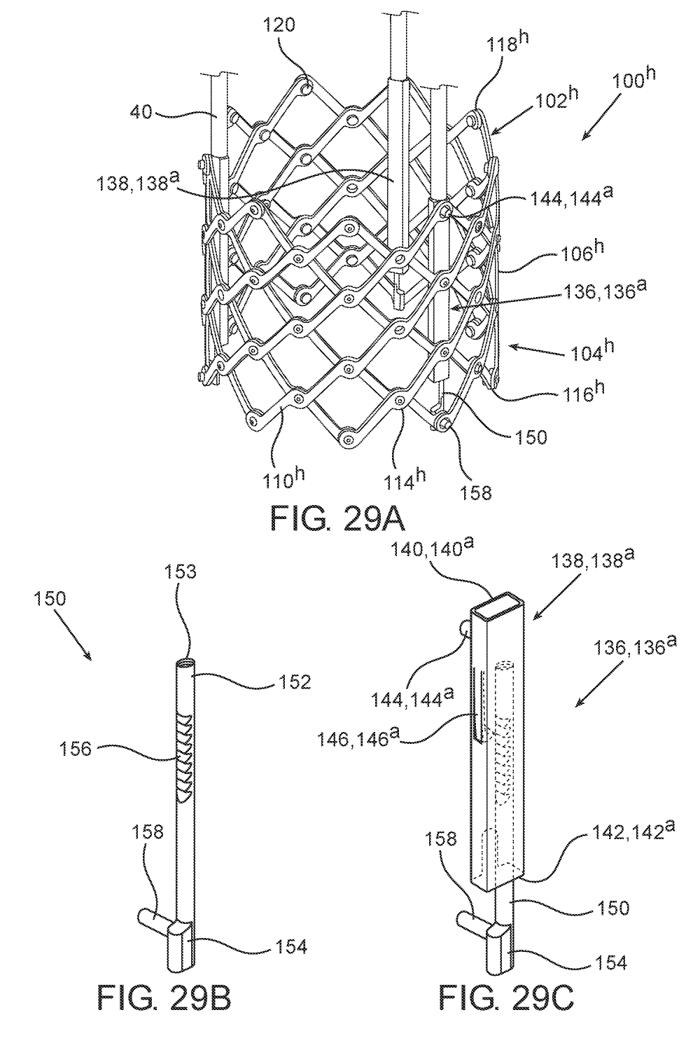

FIG. 29A schematically shows an exemplary embodiment of a mechanically expandable prosthetic valve 100^{h}, which includes a plurality of expansion and locking assemblies 136, configured to facilitate expansion of the valve 100^{h}, and in some instances, to lock the valve 100^{h} at an expanded state, preventing unintentional recompression thereof, as will be further elaborated below. Although FIG. 29A illustrates three expansion and locking assemblies 136, mounted to the frame 106^{h}, and optionally equally spaced from each other around an inner surface thereof, it should be clear that a different number of expansion and locking assemblies 136 may be utilized, that the expansion and locking assemblies 136 can be mounted to the frame 106^{h} around its outer surface, and that the circumferential spacing between expansion and locking assemblies 136 can be unequal. In some implementations, the struts of frame 106^{h} can be interconnected to each other via hinge members, such as pins 120 that can extend through apertures 115 (shown, for example, in FIG. 30A) in a manner that allows them to pivot relative to each other.

FIGS. 29B-29C show an exemplary embodiment of an expansion and locking assembly 136. An expansion and locking assembly 136 may include a hollow outer member 138, secured to a component of the valve (100^{h}), such as the frame 106^{h}, at a first location, and an inner member 150 secured to a component of the valve (100^{h}), such as the frame 106^{h}, at a second location, axially spaced from the first location.

FIG. 29B shows a view in perspective of an exemplary embodiment of an inner member 150, having an inner member proximal end portion 152 and an inner member distal end portion 154. The inner member 150 comprises an inner member fastening extension 158 extending from its distal end portion 154, which may be formed as a pin extending radially outward from the distal end portion 154, configured to be received within respective openings or apertures of struts 110^{h} intersecting at a junction 114^{h} or an apex 116^{h}, 118^{h}. The inner member 150 may further comprise a linear rack having a plurality of ratcheting teeth 156 along at least a portion of its length. According to some embodiments, one surface of the inner member 150 comprises a plurality of ratcheting teeth 156.

FIG. 29C shows the inner member 150 disposed within a lumen of an exemplary of outer member 138^{a}. The outer member 138^{a} is shown with partial transparency in FIG. 29C for sake of clarity. The outer member 138^{a} comprises an outer member proximal end portion 140^{a} defining a proximal opening of its lumen, and an outer member distal end portion 142^{a} defining a distal opening of its lumen. The outer member 138^{a} can further comprise an outer member fastening extension 144^{a} extending from its proximal end portion 140^{a}, which may be formed as a pin extending radially outward from the external surface of the proximal end portion 140^{a}, configured to be received within respective openings or apertures of struts 110^{h} intersecting at a junction 114^{h} or an apex 116^{h}, 118^{h}.

The outer member 138^{a} may further comprise a spring biased arm 146^{a}, attached to or extending from one sidewall of the outer member 138^{a}, and having a tooth or pawl at its opposite end, biased inwards toward the inner member 150 when disposed within the outer member’s lumen.

At least one of the inner or outer member 150 or 138, respectively, is axially movable relative to its counterpart. The expansion and locking assembly 136^{a} in the illustrated embodiments, comprises a ratchet mechanism or a ratchet assembly, wherein the pawl of the spring biased arm 146^{a} of the outer member 138^{a} is configured to engage with the teeth 156 of the inner member 150. The pawl of the spring biased arm 146^{a} can have a shape that is complementary to the shape of the ratcheting teeth 156, such that the pawl of the spring biased arm 146^{a} allows a sliding movement of the inner member 150 in one direction relative to the outer member 138^{a}, for example in a proximally oriented direction 2, and resists sliding movement of the inner member 150 in the opposite direction, such as a distally oriented direction, when the pawl of the spring biased arm 146^{a} is in engagement with the ratcheting teeth 156 of the inner member 150.

The spring biased arm 146^{a} can be formed of a flexible or resilient portion of the outer member 138^{a} that extends over and contacts, via its pawl, an opposing side of the outer surface of the inner member 150. According to some embodiments, the spring biased arm 146^{a} can be in the form of a leaf spring that can be integrally formed with the outer member 138^{a} or separately formed and subsequently connected to the outer member 138^{a}. The spring biased arm 146^{a} is configured to apply a biasing force against the outer surface of the inner member 150, so as to ensure that under normal operation, its pawl stays engaged with the ratcheting teeth 156 of the inner member 150.

A mechanically expandable prosthetic valve 100^{h} may be releasably attachable to at least one actuation assembly 40, and preferably a plurality of actuation assemblies 40, matching the number of expansion and locking assemblies 136. The actuation member 42 and the actuation support sleeve 46 can be movable longitudinally relative to each other in a telescoping manner to radially expand and contract the frame 106^{h}, as further described in U.S. Publication Nos. 2018/0153689, 2018/0153689 and 2018/0325665 which are incorporated herein by reference. The actuation members 42 can be, for example, wires, cables, rods, or tubes. The actuation support sleeves 46 can be, for example, tubes or sheaths having sufficient rigidity such that they can apply a distally directed force to the frame without bending or buckling.

The inner member proximal end portion 152 further comprises an inner member threaded bore 153, configured to receive and threadedly engage with a threaded portion of a distal end portion 44 (shown for example in FIG. 30C) of a corresponding actuation member 42. FIG. 29A shows a view in perspective of a valve 100^{h} in an expanded state, having its expansion and locking assemblies 136^{a} connected to actuation members 42 (hidden from view within the actuation support sleeves 46) of a delivery apparatus 12^{c}. When actuation members 42 are threaded into the inner members 150, axial movement of the actuation members 42 causes axial movement of the inner members 150 in the same direction.

According to some embodiments, the actuation assemblies 40 are configured to releasably couple to the prosthetic valve 100^{h}, and to move the prosthetic valve 100^{h} between the radially compressed and the radially expanded configurations. FIGS. 30A-30C illustrate a non-binding configuration representing actuation of the expansion and locking assemblies 136^{a} via the actuation assemblies 40 to expand the prosthetic valve 100^{h} from a radially compressed state to a radially expanded state.

FIG. 30A shows an expansion and locking assembly 136^{a}, having an outer member 138^{a}, secured to the frame 106^{h} at a first location, and an inner member 150 secured to the frame 106^{h} at a second location. According to some embodiments, the first location can be positioned at or adjacent to the outflow end 102^{h}, and the second location can be positioned at or adjacent to the inflow end portion 104^{h}. In the illustrated embodiment, the outer member 138^{a} is secured to a distal-most junction 114^{h}a which is proximal to the outflow apices 118^{h} or the outflow end 102^{h}, via outer member fastening extension 144^{a}, and the inner member 150 is secured to a proximal-most junction 114^{h}c which is proximal to the inflow apices 116^{h} or the inflow end 104^{h}, via inner member fastening extension 158. A proximal portion of the inner member 150 extends, through the distal opening of the outer member distal end 142^{a}, into the outer member’s lumen.

The expansion and locking assembly 136^{a} is shown in FIG. 30A in a radially compressed state of the frame 106^{h}, wherein the outflow and inflow apices 118^{h} and 116^{h}, respectively, are relatively distanced apart from each other along the axial direction, and the inner member proximal end portion 152 is positioned distal to the outer member proximal end portion 140^{a}.

As further shown in FIG. 30A, the actuation member distal end portion 44 is threadedly engaged with the inner member threaded bore 153. According to some embodiments, as shown in FIGS. 30A-30C, the actuation member distal end portion 44 includes external threads, configured to engage with internal threads of the inner member threaded bore 153. According to alternative embodiments, an inner member may include a proximal extension provided with external threads, configured to be received in and engage with internal threads of a distal bore formed within the actuation member (embodiments not shown).

The actuation support sleeve 46 surrounds the actuation member 42 and may be connected to the handle 30^{c} of a delivery apparatus 12^{c}. The actuation support sleeve 46 and the outer member 138^{a} are sized such that the distal lip of the actuation support sleeve 46 can abut or engage the outer member proximal end 140^{a}, such that the outer member 138^{a} is prevented from moving proximally beyond the actuation support sleeve 46.

In order to radially expand the frame 106^{h}, and therefore the valve 100^{h}, the actuation support sleeve 46 can be held firmly against the outer member 138^{a}. The actuation member 42 can then be pulled in a proximally oriented direction 2, as shown in FIG. 30B. Because the actuation support sleeve 46 is being held against the outer member 138^{a}, which is connected to the frame 106^{h} at the first location, the outflow end 102 of the frame 106^{h} is prevented from moving relative to the actuation support sleeve 46. As such, movement of the actuation member 42 in a proximally oriented direction 2 can cause movement of the inner member 150 in the same direction, thereby causing the frame 106^{h} to foreshorten axially and expand radially.

More specifically, as shown for example in FIG. 30B, the inner member fastening extension 158 extends through apertures in two struts 110^{h} interconnected at a distal junction 114^{h}c, while the outer member fastening extension 144^{a} extends through apertures in two struts 110 interconnected at a proximal junction 114^{h}a. As such, when the inner member 150 is moved axially, for example in a proximally oriented direction 2, within the outer member 138^{a}, the inner member fastening extension 158 moves along with the inner member 150, thereby causing the portion to which the inner member fastening extension 158 is attached to move axially as well, which in turn causes the frame 106^{h} to foreshorten axially and expand radially.

The struts 110^{h} to which the inner member fastening extension 158 is connected are free to pivot relative to the fastening extension 158 and to one another as the frame 106^{h} is expanded or compressed. In this manner, the inner member fastening extension 158 serves as a fastener that forms a pivotable connection between those struts 110^{h}. Similarly, struts 110^{h} to which the outer member fastening extension 144^{a} is connected are also free to pivot relative to the fastening extension 144^{a} and to one another as the frame 106^{h} is expanded or compressed. In this manner, the outer member fastening extension 144^{a} also serves as a fastener that forms a pivotable connection between those struts 110^{h}.

When the pawl of the spring biased arm 146^{a} is engaged with the ratcheting teeth 156, the inner member 150 can move in one axial direction, such as the proximally oriented direction 2, but cannot move in the opposite axial direction. This ensures that while the pawl of the spring biased arm 146^{a} is engaged with the he ratcheting teeth 156, the frame 106^{h} can radially expand but cannot be radially compressed. Thus, after the prosthetic valve 100^{h} is implanted in the patient, the frame 106^{h} can be expanded to a desired diameter by pulling the actuation member 42. In this manner, the actuation mechanism also serves as a locking mechanism of the prosthetic valve 100^{h}.

Once the desired diameter of the prosthetic valve 100^{h} is reached, the actuation member 42 may be rotated, for example in rotation direction 8, to unscrew the actuation member 42 from the inner member 150, as shown in FIG. 30C. This rotation serves to disengage the distal threaded portion 44 of the actuation member 42 from the inner member threaded bore 153, enabling the actuation assemblies 40 to be pulled away, and retracted, together with the delivery apparatus 12^{c}, from the patient’s body, leaving the prosthetic valve 100^{h} implanted in the patient. The patient’s native anatomy, such as the native aortic annulus in the case of transcatheter aortic valve implantation, may exert radial forces against the prosthetic valve 100^{h} that would strive to compress it. However, the engagement between the pawl of the spring biased arm 146^{a} and the ratcheting teeth 156 of the inner member 150 prevents such forces from compressing the frame 106^{h}, thereby ensuring that the frame 106^{h} remains locked in the desired radially expanded state.

Thus, the prosthetic valve 100^{h} is radially expandable from the radially compressed state shown in FIG. 30A to the radially expanded state shown in FIG. 30B upon actuating the expansion and locking assemblies 136^{a}, wherein such actuation includes approximating the second locations to the first locations of the valve 100^{h}. The prosthetic valve 100^{h} is further releasable from the delivery apparatus 12^{c} by decoupling each of the actuation assemblies 40 from each of the corresponding expansion and locking assemblies 136^{a} that were attached thereto.

While the frame 106^{h} is shown above to expand radially outward by axially moving the inner member 150 in a proximally oriented direction 2, relative to the outer member 138, it will be understood that similar frame expansion may be achieved by axially pushing an outer member 138 in a distally oriented direction, relative to an inner member 150.

While a threaded engagement is illustrated and described in the above embodiments, serving as an optional reversible-attachment mechanism between the actuation assemblies 40 and the inner members 150, it is to be understood that in alternative implementations, other reversible attachment mechanisms may be utilized, configured to enable the inner member 150 to be pulled or pushed by the actuation assemblies 40, while enabling disconnection there-between in any suitable manner, so as to allow retraction of the delivery apparatus from the patient’s body at the end of the implantation procedure.

While a specific actuation mechanism is described above, utilizing a ratcheting mechanism between the inner and the outer members of the expansion and locking assemblies 136, other mechanisms may be employed to promote relative movement between inner and outer members of actuation assemblies, for example via threaded or other engagement mechanisms. Further details regarding the structure and operation of mechanically expandable valves and delivery system thereof are described in US Patent No. 9,827,093, U.S. Pat. Application Publication Nos. 2019/0060057, 2018/0153689 and 2018/0344456, and U.S. Pat. Application Nos. 62/870,372 and 62/776,348, all of which are incorporated herein by reference.

According to some embodiments, a prosthetic valve 100^{h} comprises at least one adjustable commissure support 160^{h} provided with a clamp 184, coupled to the frame 106^{h}, wherein at least one outer member 138 of an expansion and locking assembly 140 is configured to snap-fit or clip into the at least one clamp 184 of the adjustable commissure support 160^{h}.

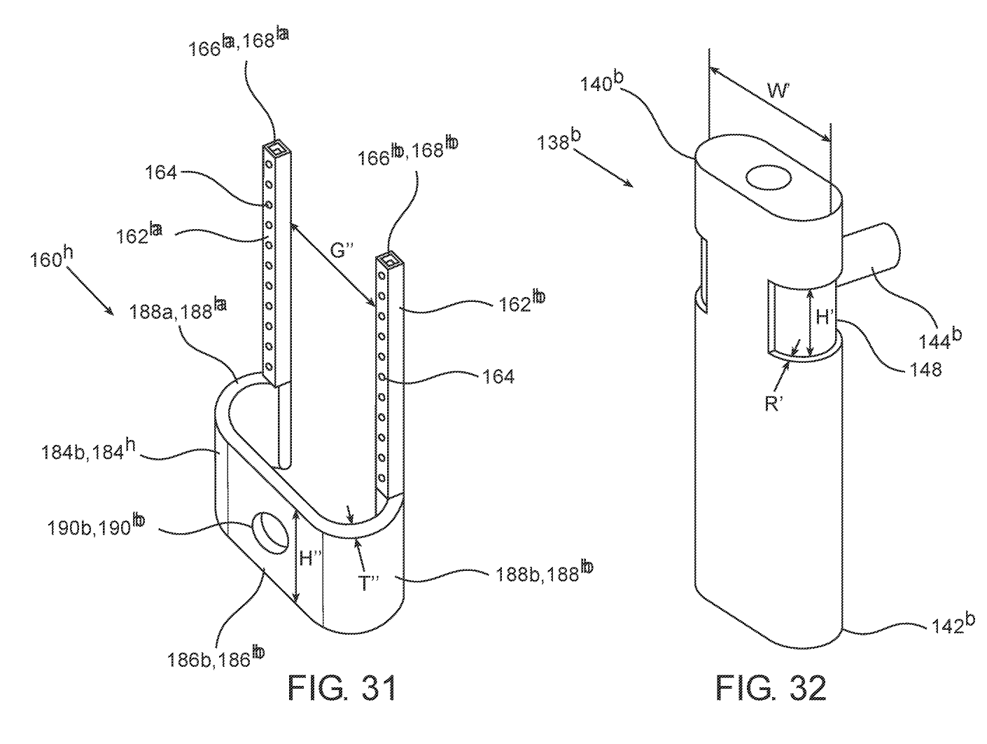

FIG. 31 shows a view in perspective of an adjustable commissure support 160^{h}, in accordance with some applications of the current invention. An adjustable commissure support 160^{h} comprises a clamp 184 having a clamp mid-portion 186, and a couple of inwardly biased opposing side arms 188 extending in a continuous manner from both sides of the mid-portion 186. The adjustable commissure support 160^{h} further comprise a couple of adjustable arms 162^{h}, extending proximally from the free ends of the side arms 188. As illustrated, the side arms 188 may be arched inward, wherein the adjustable arms 162^{h} define a gap G″ there-between, through which an outer member 138 of an expansion and locking assembly 136, may pass toward the clamp mid-portion 186.

According to some embodiments, the clamp mid-portion 186 comprises an opening 190, configured to receive a fastener that may extend therethrough, such as an outer member fastening extension 144. FIG. 31 illustrates an exemplary embodiment of a clamp 184 comprising an opening 190 extending through the thickness of the clamp mid-portion 186. The side arms 188a and 188b are continuously arching from the clamp mid-portion 186 toward each other at their opposite free ends, defining a gap G″ between the adjustable arms 162^{h} extending therefrom. In some implementations, such as that of clamp 184 shown in FIG. 31, the clamp mid-portion 186 may be substantially flat.

The gap G″ can be dimensioned to allow tabs having a thickness T′ to extend therethrough, and optionally accommodate several layers of the tabs 132, in the same manner described hereinabove with respect to the gap G′ of FIG. 6.

FIG. 32 shows an exemplary embodiment of an outer member 138^{b}, configured to be used in combination with an adjustable commissure support 160^{h}, according to some implementation of the current application. The outer member 138^{b} includes an outer member coupling recess 148. The outer member coupling recess 148 spans across the sidewalls of the outer member 138^{b}, and optionally along at least a portion of its inner wall (i.e., the wall facing the frame 106^{h}). The outer member coupling recess 148 is configured to accommodate at least a portion of a clamp 184 therein. The outer member coupling recess 148 shown in FIG. 32 is shaped so as to closely match the shape of the clamp 184 shown in FIG. 31.

The side arms 188 are resiliently expandable away from each other, such that the outer member coupling recess 148 is passable through the gap G″ formed between the adjustable arms 162^{h} extending therefrom. In use, the outer member 138^{b} may be pushed into the clamp 184, having the outer member coupling recess 148 aligned with the clamp 184. The gap G″ is smaller than the maximal lateral width W′ of the outer member 138^{b} (width W′ may be measured at the region of the outer member coupling recess 148). The sidewalls of the outer member 138^{b}, at the region of the recess 148, may apply a force sufficient to expand the side arms 188 away from each other, so as to allow the outer member 138^{b} to pass therethrough radially outward, toward the clamp mid-portion 186. Once the outer member coupling recess 148 is completely accommodated within the clamp 184, and in the absence of further expanding force applied to the side arms 188, the side arms resiliently snap back toward each other to compress against the outer member coupling recess 148, in order to lock in place the outer member 138^{b} with respect to the clamp 184.

In some implementations, the height H′ of the outer member coupling recess 148, is substantially equal to the height H″ of the clamp 184, such that when the outer member 138^{b} is engaged with the clamp 184, axial movement between the outer member 138^{b} and the adjustable commissure support 160^{h} is prevented.

The term “substantially equal”, as used herein, means within a range of no more than ±10 percent of the referred measure. For example, the height H′ of the outer member coupling recess 148 being substantially equal to the height H″ of the clamp 184, means that height H′ is not greater than 110% of the height H″.

In some implementations, the depth R′ of the outer member coupling recess 148 is substantially equal to the thickness T′ of the clamp 184, such that when the clamp 184 is engaged with the outer member 138^{b}, the clamp’s outer surface is flush with the outer surface of the outer member (138^{b}) around the recess 148.

It is to be understood that features of adjustable commissure supports 160^{h} that include adjustable arm 162^{h} extending from side arms 188 of a clamp 184 can be combined with any other features of any other type of adjustable commissure supports 160^{a}, 160^{b}, 160^{c}, and/or 160^{d} described hereinabove, and include two adjustable arm 162 for each adjustable commissure supports 160. For example, the engagement portions 166^{h} can comprise sockets 168^{h} as illustrated in FIG. 31, or may be alternatively formed as drive-screw couplers, similar to engagement portion 166^{d} illustrated in FIGS. 23A-C. Similarly, the engagement portions 166^{h} can have a plurality of apertures 164, as disclosed hereinabove throughout the specification for all other types of adjustable arm 162.

FIG. 31 shows an exemplary embodiment of an adjustable commissure supports 160 having the adjustable arms 162^{h} rigidly attached to the side arms 188^{h} of the clamp 184^{h}, to enable twisting of the adjustable arms 162^{h} between the engagement portions 166^{h} and the side arms 188^{h}, similar to the twistable configuration illustrated in FIGS. 9A-9B.

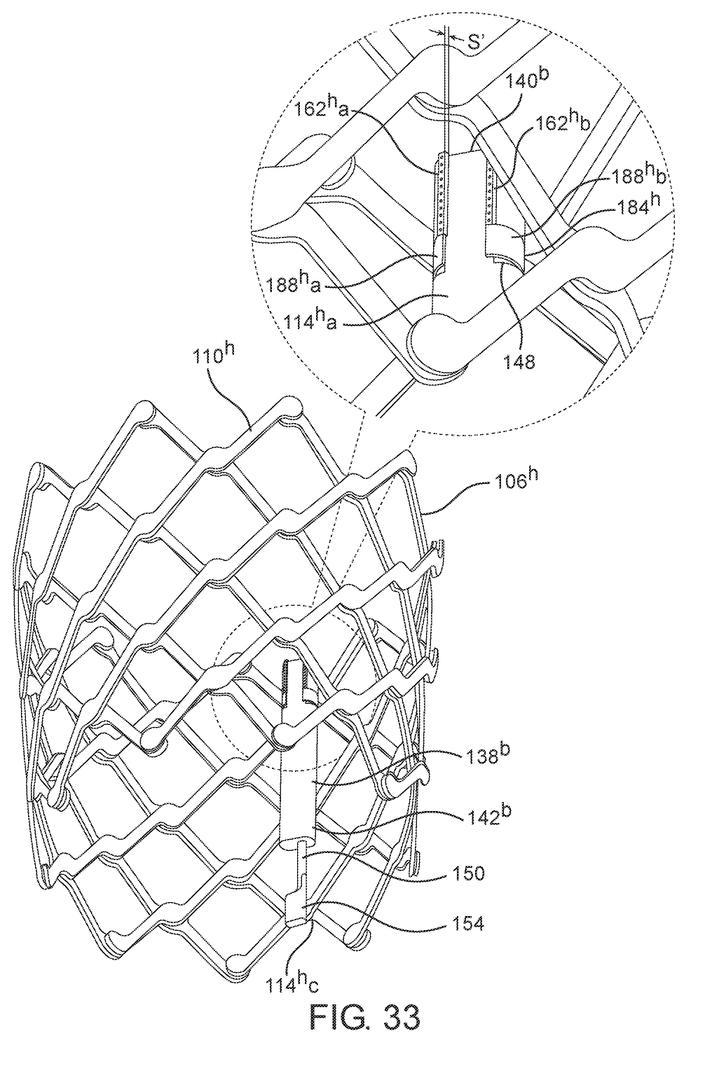

FIG. 33, shows an expansion and locking assembly (136) coupled to the frame 106^{h}. The outer member 138^{b} of FIG. 32 is coupled to the first location of the frame 106^{h}, for example, via its outer member fastening extension 144^{b}, while engaged with the adjustable commissure support 160^{h} clamped over the outer member c recess 148. As shown, the adjustable arms 162^{h} are offset radially inward with respect to the outer member 138^{b}, by an offsetting gap S′. In some implementations, the extent of the offsetting gap S′ is at least equal to, or may be larger than, the thickness T′ of the tabs (132), allowing the tabs (132) to extend through the offsetting gap S′ when wrapped around the adjustable arms 162^{h}. As noted above, the tabs (132) may be wound around the adjustable arms 162^{h} in a manner that result in several layers of the tabs wound therearound. In some embodiments, the offsetting gap S′ is at least twice as large as the thickness T′ of the tabs. In some embodiments, the offsetting gap S′ is at least three times as large as the thickness T′ of the tabs. In some embodiments, the offsetting gap S′ is at least four times as large as the thickness T′ of the tabs.

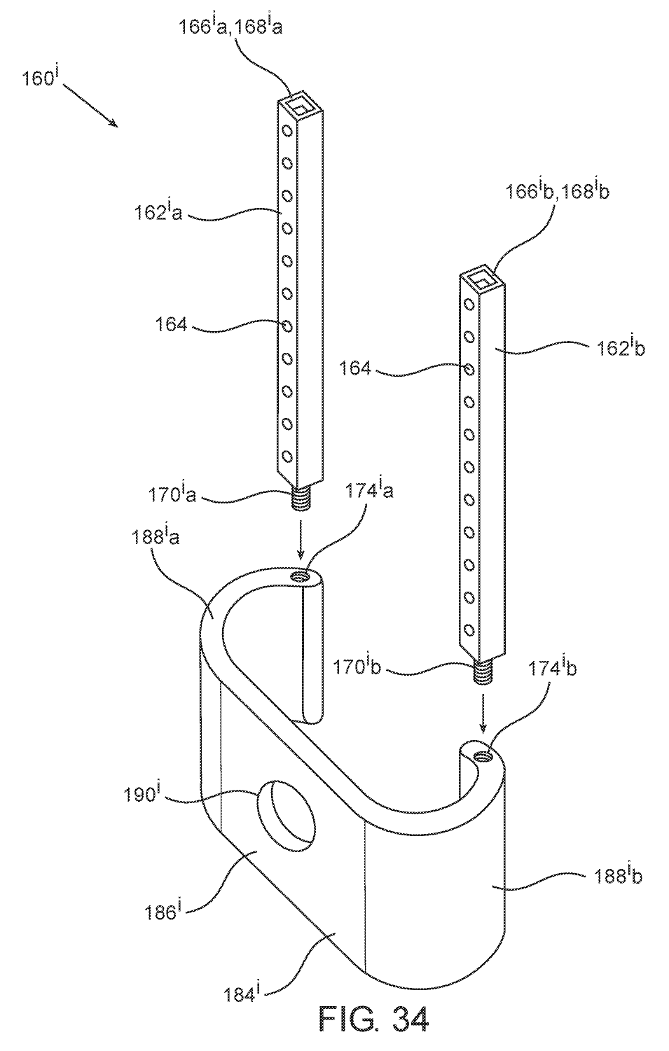

FIG. 34 shows an exemplary embodiment of an adjustable commissure supports 160ⁱ which identical to adjustable commissure supports 160^{h}, except that the adjustable arms 162ⁱ are rotatably coupled to the side arms 188ⁱ of the clamp 184ⁱ, enabling them to rotate in a similar manner to that illustrated for adjustable arms 162^{b} in FIGS. 10A-10B, wherein the base threaded portions 174ⁱ are comprised in the side arms 188ⁱ.

In some implementations, the adjustable commissure supports 160^{h}, 160ⁱ can be used in combination with a commissure adjustment assembly 200^{a} that includes primary pinion gears 214^{a} and secondary pinion gears 216^{a}, in a similar manner to that described hereinabove with respect to FIGS. 11-13, mutatis mutandis. In some implementations, the adjustable commissure supports 160^{h}, 160ⁱ can be used in combination with any type of an adjustment assembly 50 as long as each adjustment assembly 50 includes two adjustment arms 52, and in a similar manner to that described hereinabove with respect to FIGS. 22A-23C, mutatis mutandis.

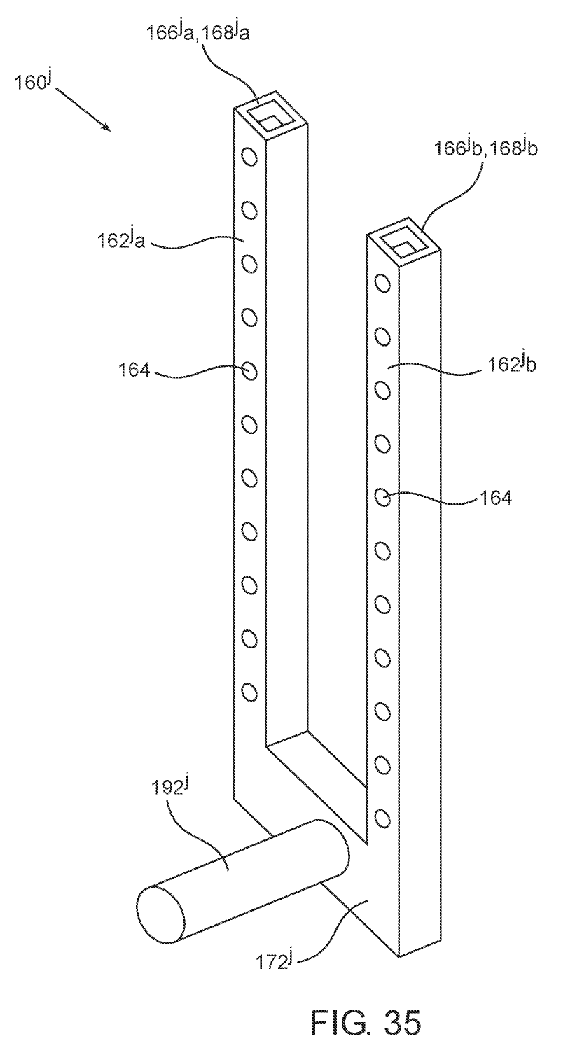

FIG. 35 shows another type of an adjustable commissure support 160ⁱ, in accordance with some implementation of the current invention. Adjustable commissure support 160^{j} may be similar adjustable commissure support 160^{a} described hereinabove with respect to FIGS. 9A-9B, except that the adjustable commissure support 160^{j} further comprises a commissure support fastening extension 192^{j}, extending radially from the support base 172^{j}.

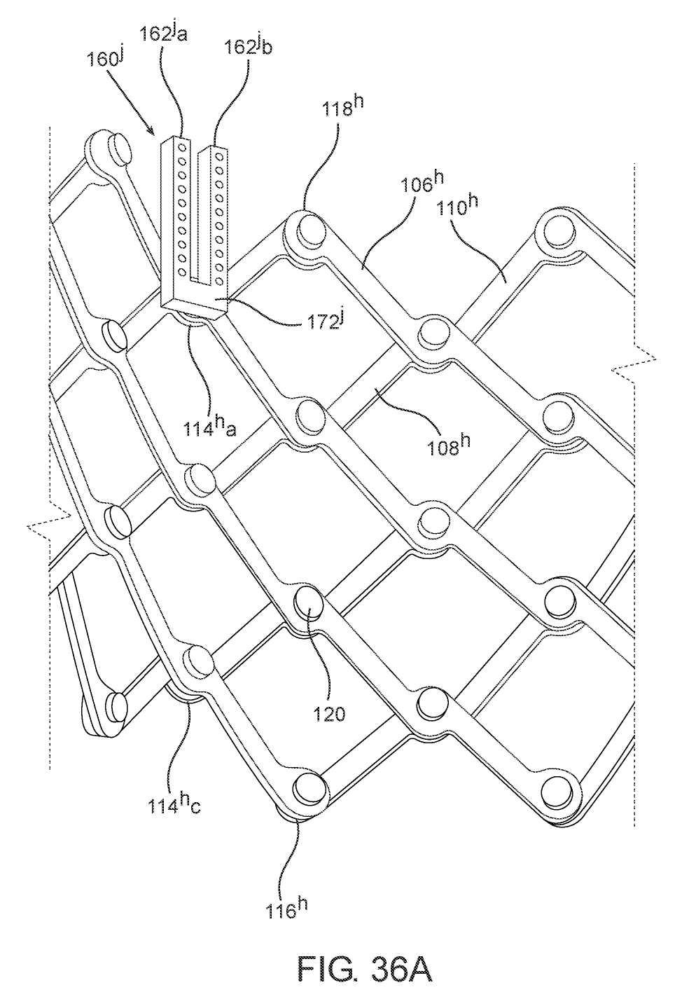

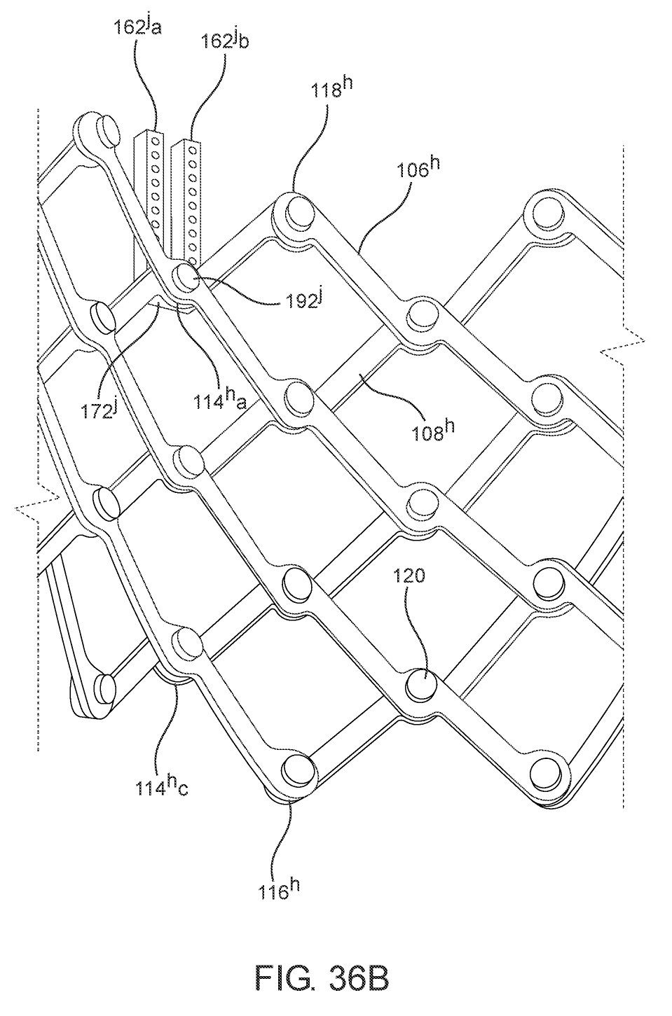

FIGS. 36A-36B show two exemplary configurations in which the adjustable commissure support 160^{j} can be coupled to a frame 106, such as a frame 106^{h} of mechanically expandable valve 100^{h}, by extending the commissure support fastening extension 192^{j} through apertures formed at a junction 114^{h} between two intersecting struts 110^{h}. According to some embodiments, the adjustable commissure support 160^{j} can be coupled to the frame 106 such that the adjustable arms 162^{h} are positioned radially inward with respect to the frame 106, as shown in FIG. 36A. According to some embodiments, the adjustable commissure support 160^{j} can be coupled to the frame 106 such that the adjustable arms 162^{h} are positioned radially outward with respect to the frame 106, as shown in FIG. 36B.

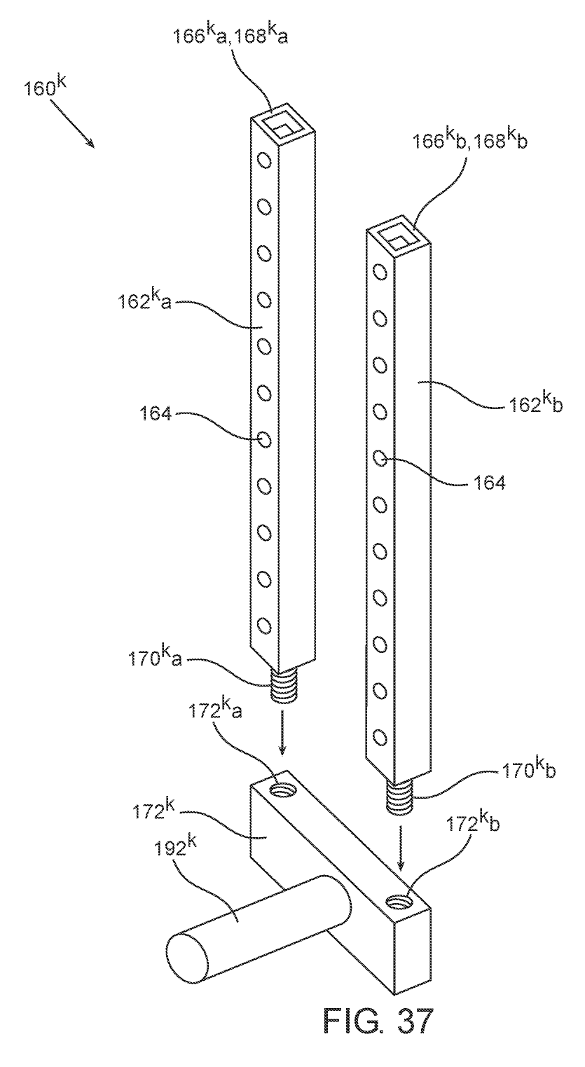

FIG. 37 shows an exemplary embodiment of an adjustable commissure supports 160^{k} which identical to adjustable commissure supports 160^{j}, except that the adjustable arms 162^{k} are rotatably coupled to the support base 172^{k}, enabling them to rotate in a similar manner to that illustrated for adjustable arms 162^{b} in FIGS. 10A-10B.

Reference is made to FIGS. 31, 34, 35 and 37. It is to be noted that the commissure support fastening extension 192^{j} or 192^{k} shown in FIGS. 35 and 37, can be combined with the adjustable commissure supports 160^{h} and 160ⁱ shown in FIGS. 31 and 34, such that any of the adjustable commissure supports 160^{h} and 160ⁱ can be provided with a commissure support fastening extension 192 extending radially outward from the clamp mid-portion 186^{h} and 186ⁱ, respectively, instead of an opening 190.

In such embodiments, the outer member (138) will be devoid of an outer member fastening extension (144). The adjustable commissure supports (160) will be attached to the frame (106) by insertion of the commissure support fastening extension (192) into apertures formed within a junction (114) of the frame (106), similar to the attachment configuration shown in FIG. 36A, wherein the clamp (184) and the adjustable arms (162) are positioned radially inward with respect to the frame (106). The outer member (138) will then be clamped along its outer member coupling recess (148) to the clamp (184), thereby attaching the outer member (138), which is devoid of its own fastening extension, to the frame at a first location -via the adjustable commissure support.

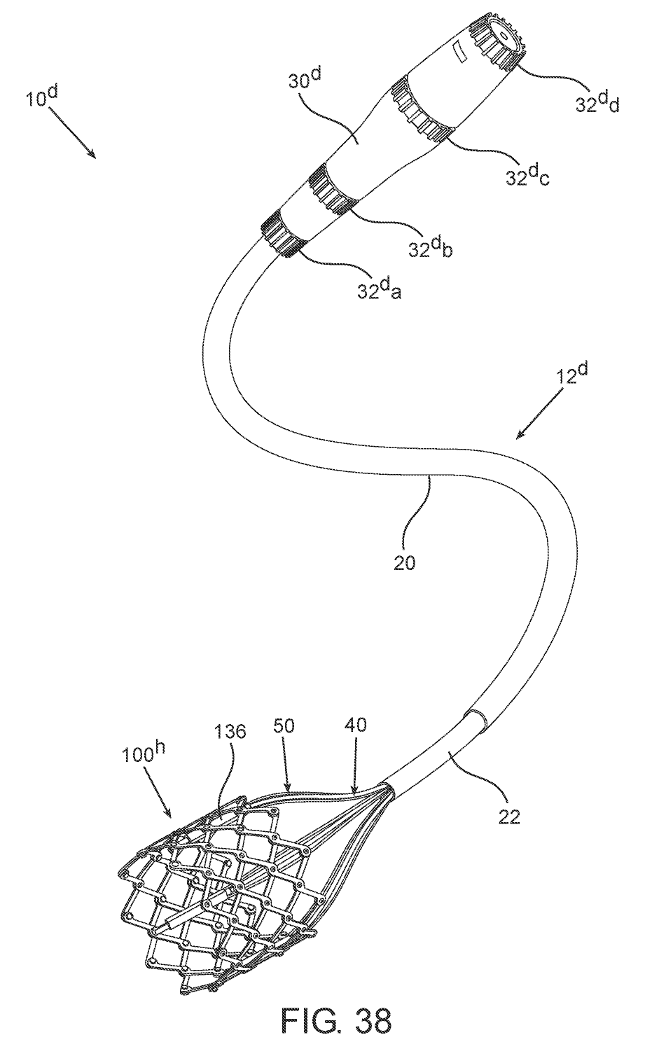

FIG. 38 shows a view in perspective of another type of a delivery assembly 10^{d} that can be used for delivering and implanting mechanically expandable prosthetic valve 100^{h}, according to some embodiments. The delivery apparatus 12^{d} of the delivery assembly 10^{d} can similar to the delivery apparatus 12^{c} described hereinabove with respect to FIG. 28, which includes the actuation assemblies 40, except that it may further include the adjustment assemblies 50, described hereinabove with respect to FIG. 21. In some embodiments, both the actuation assemblies 40 and the adjustment assemblies 50 extend through the same delivery shaft 22.

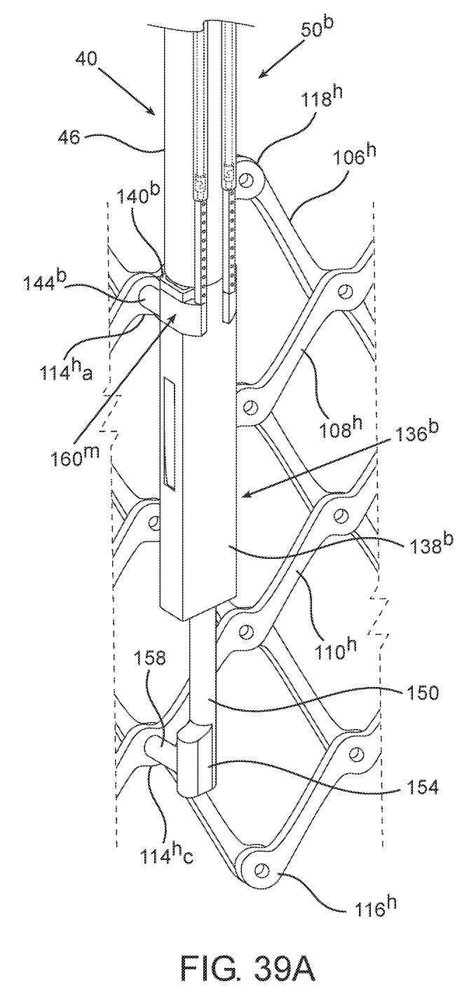

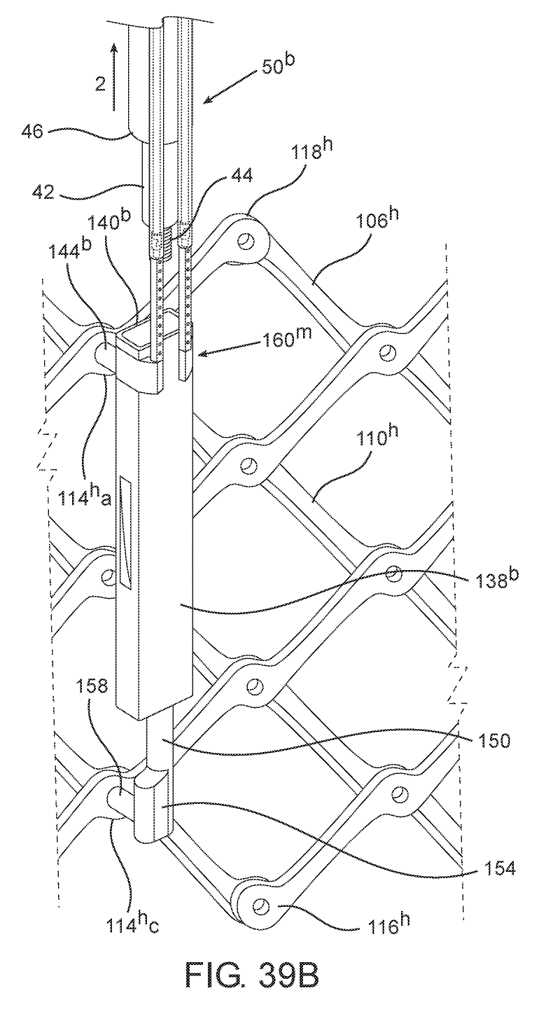

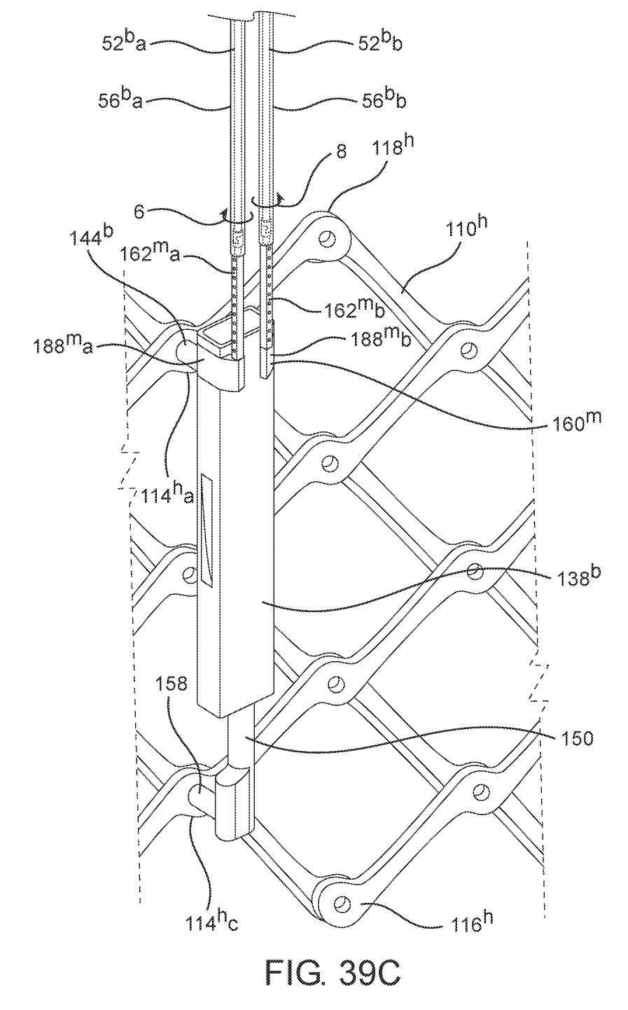

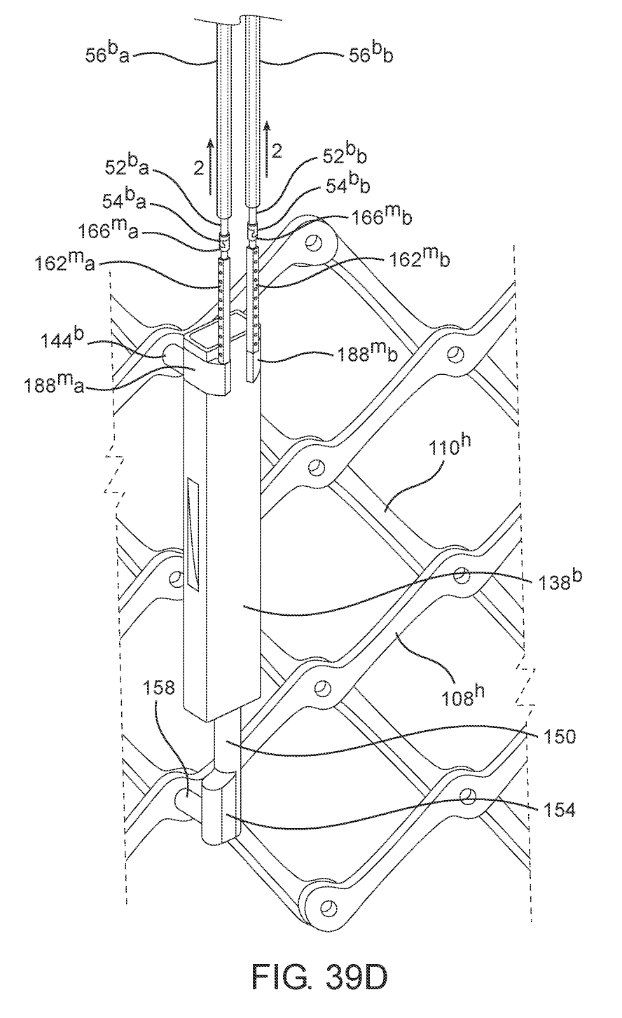

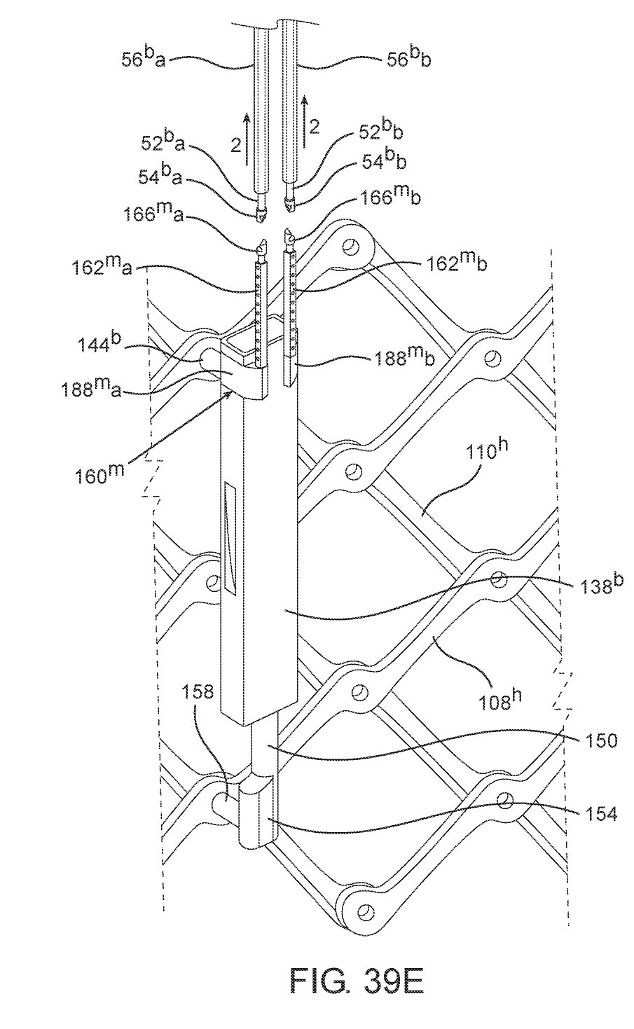

FIGS. 39A-39E illustrate a non-binding configuration representing actuation of the expansion and locking assemblies 136^{b} via the actuation assemblies 40 to expand the prosthetic valve 100^{h} from a radially compressed state to a radially expanded state, in a manner similar to that described above with respect to FIGS. 30A-30C, followed by utilization of the adjustment assemblies 50 to rotate or twist the adjustable arms 162^{m} of adjustable commissure support 160^{m}, in a manner similar to that described above with respect to FIGS. 22A-23C.

The adjustable arms 162^{m} comprise, as illustrated throughout FIGS. 39A-39E, engagement portions 166^{e} formed as drive-screw couplers, and the adjustment assemblies 50^{b} are shown to include driving heads 54^{b} at the distal ends of the adjustment arms 52^{b}, formed as complementary shaped drive-screw couplers. However, it is to be understood that this engagement is shown by way of illustration and not limitation, and that the steps that will be described with respect to FIGS. 39A-39E can be similarly implemented for any other type of engagement between the adjustment assemblies 50 and the adjustable commissure support 160, such as driving heads 54 shaped as non-cylindrical extension, engaged with sockets 168 of the adjustable arms 162.

FIG. 39A shows an expansion and locking assembly 136^{b}, having an outer member 138^{b}, secured to the frame 106^{h} at a first location, and an inner member 150 secured to the frame 106^{h} at a second location. An adjustable commissure support 160^{m} is clamped, via a clamp 184^{m} thereof, over the outer member coupling recess 148. The adjustable commissure support 160^{m} can be any of the adjustable commissure support 160^{h} described hereinabove with respect to FIG. 31, or the adjustable commissure support 160ⁱ described hereinabove with respect to FIG. 34, and may be used in combination with the outer member 138^{b} in a manner similar to that described hereinabove with respect to FIG. 33.

The driving heads 54^{b} of the adjustment arms 52^{b} are releasably engaged with the engagement portions 166^{m} of the adjustable arms 162^{m}, while the adjustment sleeves 56^{m} cover, in the illustrated configuration, both the driving heads 54^{b} and the engagement portions 166^{m}, so as to prevent spontaneous disengagement therebetween.

While the outer member 138^{b} is illustrated in FIGS. 39A-39E as having an outer member fastening extension 144^{b}, that may extend through an opening (190) of the clamp 184^{m}, thereby coupling both to the junction 114^{h}a of the frame 106^{h}, it is to be understood that alternatively, the series of steps that will be described with respect to FIGS. 39A-39E, can be applied to an outer member (138) which is devoid of an outer member fastening extension, and is clamped at the outer member coupling recess (148) to the clamp (184^{m}) of an adjustable commissure support (160^{m}), wherein the clamp (184^{m}) includes a commissure support fastening extension (192) extending radially outward therefrom, and coupled to the junction 114^{h}a of the frame 106^{h} in a similar manner.

The expansion and locking assembly 136^{b} is shown in FIG. 39A in a radially compressed state of the frame 106^{h}, wherein the outflow and inflow apices 118^{h} and 116^{h}, respectively, are relatively distanced apart from each other along the axial direction, and the inner member proximal end portion 152 is positioned distal to the outer member proximal end portion 140^{b}.

The actuation support sleeve 46 surrounds the actuation member 42 and may be connected to the handle 30^{d} of a delivery apparatus 12^{d}. In order to radially expand the frame 106^{h}, and therefore the valve 100^{h}, the actuation support sleeve 46 can be held firmly against the outer member 138^{b}. The actuation member 42 can then be pulled in a proximally oriented direction 2. Because the actuation support sleeve 46 is being held against the outer member 138^{b}, which is connected to the frame 106^{h} at the first location, the outflow end 102 of the frame 106^{h} is prevented from moving relative to the actuation support sleeve 46. As such, movement of the actuation member 42 in a proximally oriented direction 2 can cause movement of the inner member 150 in the same direction, thereby causing the frame 106^{h} to foreshorten axially and expand radially.

Once the desired diameter of the prosthetic valve 100^{h} is reached, the actuation member 42 may be rotated, to unscrew the actuation member 42 from the inner member 150. This rotation serves to disengage the distal threaded portion 44 of the actuation member 42 from the inner member threaded bore 153, enabling the actuation assemblies 40 to be pulled away, and retracted, as shown in FIG. 39B, together with the delivery apparatus 12^{c}, from the patient’s body, leaving the prosthetic valve 100^{h} implanted in the patient.

At this stage, the actual expansion diameter of the frame 106^{h} can be determined or estimated, based on various typed of expansion diameter indications method, including, but not limited to, imaging modalities such as fluoroscopy or ultrasound. Once a determination is made regarding the actual expansion diameter of the frame 106^{h}, the clinician may decide whether leaflets tension should be adjusted according to this diameter. If adjustment is required, the adjustment assemblies 50^{b} can be utilized to adjust the tension of the leaflet (126) mounted within the expanded valve, so as to allow proper coaptation thereof.

In the exemplary illustration of FIG. 39C, both adjustment arms 52^{b} are rotated in opposite rotation directions. For example, the adjustment arm 52^{b}a is rotated in first rotational direction 6 (e.g., clockwise), while the adjacent adjustment arm 52^{b}b is rotated in an opposite, second rotational direction 8 (e.g., counterclockwise), to rotate the engagement portion 166^{m}a and 166^{m}b of the adjustable arms 162^{m}a and 162^{m}b in the same directions. This opposite directions of rotation applied to the adjustment arms 52^{b} are desirable in case the tabs (132) are wrapped around the adjustable arms 162^{m} in opposite direction, which will result in the tabs being wound around the respective adjustable arms 162^{m} in opposite directions as well, similar to the configuration described hereinabove with respect to FIGS. 7A-7B, during rotational movement of the adjustment arm 52^{b}.

It is to be understood that in alternative configurations, wherein both tabs (132) are wrapped around the respective adjustable arms (162^{m}) in the same direction, the adjustment arms 52^{b}a and 52^{b}b will be rotated in the same rotational direction.

FIGS. 39A-39E illustrate the adjustment assembly 50^{b} used in combination with an adjustable commissure support 160^{m} having both of the adjustable arms 162^{m} rotatably coupled to the side arms 188^{m} of the clamp 184^{m}, such that once the engagement portions 166^{m}a and 166^{m}b are rotated, the adjustable arms 162^{m}a and 162^{m}b are rotated therewith, relative to the side arms 188^{m}. It is to be understood that the adjustment assembly 50^{b} can be used in combination with an adjustable commissure support 160^{m} which is similar to adjustable commissure support 160^{h}, having both of the adjustable arms (162) rigidly attached to the side arms (188), in the same manner, mutatis mutandis. In this case, similar rotational movement of the engagement portions 166^{m}a and 166^{m}b, resulting from the rotation of the adjustment arms 52^{b}a and 52^{b}b, will serve to twist the adjustable arms 162^{m}a and 162^{m}b between their engagement portions 166^{m} and the side arms 188^{m}, similar to the configuration of FIGS. 9A-9B.

Once rotation of the adjustment arms 52^{b} is completed, the adjustment sleeves 56^{b} are pulled proximally 2, as shown in FIG. 39D, so as to expose the driving heads 54^{b} and the engagement portion 166^{m}, which will allow them to disengage from each other. As shown in FIG. 39E, once the adjustment assemblies 50^{b} are disengaged, they can be further pulled away and retracted, with the rest of the delivery apparatus 12^{d}, from the patient’s body, leaving the prosthetic valve (100) implanted in the patient.

While adjustment of the adjustable commissure support 160^{m} is illustrated in FIGS. 39A-39E to be performed after disengagement and retraction of the actuation assemblies 40, it is to be understood that the steps of disengagement and retraction of the actuation assemblies 40 can be alternatively performed in unison with disengagement and retraction of the adjustment assemblies 50, or after disengagement and retraction of the adjustment assemblies 50.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
a plurality of adjustable commissure supports, each comprising: a support base attached to the frame, and at least one adjustable arm extending proximally from the support base, wherein each adjustable arm comprises an engagement portion;
wherein the tabs are attached to the adjustable arms;
wherein the adjustable arms are configured to rotate or twist when rotational force is applied to the engagement portions; and
wherein rotation or twisting of the adjustable arms causes the tabs attached thereto, to wind around the adjustable arms.

Example 2. The prosthetic valve of any example herein, particularly example 1, wherein the support base is integrally formed with the frame.

Example 3. The prosthetic valve of any example herein, particularly any one of examples 1 to 2, wherein the tabs are wrapped over the adjustable arms.

Example 4. The prosthetic valve of any example herein, particularly any one of examples 1 to 3, wherein each adjustable arm has a non-circular cross section.

Example 5. The prosthetic valve of any example herein, particularly any one of examples 1 to 4, wherein each adjustable arm comprises a plurality of apertures.

Example 6. The prosthetic valve of any example herein, particularly example 5, wherein the apertures are axially spaced apart from each other.

Example 7. The prosthetic valve of any example herein, particularly any one of examples 5 to 6, wherein the tabs are sutured to the adjustable arms via sutures through the tabs and the apertures.

Example 8. The prosthetic valve of any example herein, particularly example 7, wherein the sutures extend through portions of the tab disposed over opposite sides of the adjustment arms.

Example 9. The prosthetic valve of any example herein, particularly any one of examples 1 to 8, wherein the engagement portion comprises a non-cylindrical socket.

Example 10. The prosthetic valve of any example herein, particularly any one of examples 1 to 8, wherein the engagement portion comprises a proximally oriented extension formed as a drive-screw coupler.

Example 11. The prosthetic valve of any example herein, particularly example 10, wherein the engagement portion comprises an angled surface.

Example 12. The prosthetic valve of any example herein, particularly any one of examples 1 to 1, wherein the adjustable arms are rigidly attached to the support base.

Example 13. The prosthetic valve of any example herein, particularly any one of examples 1 to 1, wherein the adjustable arms are integrally formed with the support base.

Example 14. The prosthetic valve of any example herein, particularly any one of examples 12 to 13, wherein the adjustable arms are configured to twist between the support base and the engagement portions, upon application of rotational force to the engagement portions.

Example 15. The prosthetic valve of any example herein, particularly example 14, wherein the adjustable arms comprise a plastically deformable material.

Example 16. The prosthetic valve of any example herein, particularly any one of examples 1 to 11, wherein the adjustable arms are rotationally coupled to the support base.

Example 17. The prosthetic valve of any example herein, particularly example 16, wherein the adjustable arms are threadedly coupled to the support base.

Example 18. The prosthetic valve of any example herein, particularly example 17, wherein the support base comprises at least one base threaded portion, and wherein each adjustable arm comprises an adjustable arm threaded portion, configured to threadedly engage with the base threaded portion.

Example 19. The prosthetic valve of any example herein, particularly any one of examples 1 to 18, wherein the support base further comprises at least one offsetting extension, extending radially therefrom, and wherein the at least one adjustable arm extends from the respective offsetting extension.

Example 20. The prosthetic valve of any example herein, particularly example 19, wherein the at least one offsetting extension extends radially away from the support base.

Example 21. The prosthetic valve of any example herein, particularly example 19, wherein the at least one offsetting extension extends radially inward with respect to the support base.

Example 22. The prosthetic valve of any example herein, particularly any one of examples 19 to 21, wherein the offsetting extension is integrally formed with the support base.

Example 23. The prosthetic valve of any example herein, particularly any one of examples 1 to 18, wherein each adjustable commissure support further comprises a commissure support fastening extension.

Example 24. The prosthetic valve of any example herein, particularly example 23, wherein the adjustable commissure supports are attached to the frame via the commissure support fastening extensions, such that the adjustable arms are positioned radially inward with respect to the frame.

Example 25. The prosthetic valve of any example herein, particularly any one of examples 1 to 24, wherein each adjustable commissure support comprises a single adjustable arm.

Example 26. The prosthetic valve of any example herein, particularly example 25, wherein two tabs of adjacent leaflets of the leaflet assembly are attached to the adjustable commissure support, such that both tabs are wrapped over the single adjustable arm.

Example 27. The prosthetic valve of any example herein, particularly example 26, wherein the leaflets are asymmetric leaflets, each asymmetric leaflet having a short tab and an opposite long tab, and wherein the tabs of adjacent leaflets are wrapped over the single adjustable arm such that the short tab of one leaflet is wrapped around the adjustable arm, in direct contact therewith, and the long tab of the adjacent leaflet is wrapped over the short tab.

Example 28. The prosthetic valve of any example herein, particularly example 27, wherein the length of the long tab is at least 10% longer than the length of the short tab.

Example 29. The prosthetic valve of any example herein, particularly example 27, wherein the length of the long tab is at least 20% longer than the length of the short tab.

Example 30. The prosthetic valve of any example herein, particularly example 27, wherein the length of the long tab is at least 50% longer than the length of the short tab.

Example 31. The prosthetic valve of any example herein, particularly example 27, wherein the length of the long tab is at least 100% longer than the length of the short tab.

Example 32. The prosthetic valve of any example herein, particularly any one of examples 1 to 24, wherein each adjustable commissure support comprises two adjustable arms that are laterally spaced from each other and define a gap therebetween.

Example 33. The prosthetic valve of any example herein, particularly example 32, wherein two tabs of adjacent leaflets of the leaflet assembly are attached to the adjustable commissure support, such that each tab is wrapped over one of the adjustable arms.

Example 34. The prosthetic valve of any example herein, particularly example 33, wherein both tabs are wrapped over the respective adjustable arms in opposite directions with respect to each other.

Example 35. The prosthetic valve of any example herein, particularly any one of examples 32 to 34, wherein the gap is at least two times larger than the thickness of the tabs.

Example 36. The prosthetic valve of any example herein, particularly any one of examples 32 to 34, wherein the gap is at least four times larger than the thickness of the tabs.

Example 37. The prosthetic valve of any example herein, particularly any one of examples 32 to 34, wherein the gap is at least six times larger than the thickness of the tabs.

Example 38. The prosthetic valve of any example herein, particularly any one of examples 32 to 34, wherein the gap is at least eight times larger than the thickness of the tabs.

Example 39. A prosthetic valve comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
a plurality of adjustable commissure supports, each comprising:
   a support base attached to the frame;
   two adjustable arms coupled to the support base, and rotatable relative thereto, wherein each adjustable arm comprises an engagement portion; and
   two scallop adjustment arms, wherein each scallop adjustment arm extends continuously from one of the adjustable arms;
wherein the tabs of adjacent leaflets are attached to the adjustable commissure supports, such that each tab is attached to one of the adjustable arms;
wherein portions of the cusp edge of each leaflet are attached to scallop adjustment arms of adjustable commissure supports on both sides of the leaflet;
wherein both adjustable arms and scallop adjustment arms of a single adjustable commissure supports are configured to rotate in opposite directions, when opposite rotational forces are applied to their engagement portions; and
wherein rotation of the adjustable arms and the scallop adjustment arms causes the tabs to wind around the adjustable arms they are attached to, and causes the portions of the cusp edges to wind around the scallop adjustment arms they are attached to.

Example 40. The prosthetic valve of any example herein, particularly example 39, wherein the support base is integrally formed with the frame.

Example 41. The prosthetic valve of any example herein, particularly any one of examples 39 to 40, wherein each adjustable arm and the scallop adjustment arm extending therefrom are integrally formed.

Example 42. The prosthetic valve of any example herein, particularly any one of examples 39 to 41, wherein the scallop adjustment arm comprises a torque transmitting shaft.

Example 43. The prosthetic valve of any example herein, particularly any one of examples 39 to 42, wherein the support base comprises two eyelets through which the adjustable arms and/or the scallop adjustment arms extend.

Example 44. The prosthetic valve of any example herein, particularly any one of examples 39 to 42, wherein the frame comprises eyelets through which the scallop adjustment arms extend.

Example 45. The prosthetic valve of any example herein, particularly any one of examples 43 to 44, wherein the eyelets are extending radially inward.

Example 46. The prosthetic valve of any example herein, particularly any one of examples 43 to 45, wherein the eyelets comprises internal threads.

Example 47. The prosthetic valve of any example herein, particularly any one of examples 39 to 46, wherein the tabs are wrapped over the adjustable arms.

Example 48. The prosthetic valve of any example herein, particularly any one of examples 39 to 47, wherein each adjustable arm comprises a plurality of apertures.

Example 49. The prosthetic valve of any example herein, particularly example 48, wherein the apertures are axially spaced apart from each other.

Example 50. The prosthetic valve of any example herein, particularly any one of examples 48 to 49, wherein the tabs are sutured to the adjustable arms via sutures through the tabs and the apertures.

Example 51. The prosthetic valve of any example herein, particularly example 50, wherein the sutures extend through portions of the tab disposed over opposite sides of the adjustment arms.

Example 52. The prosthetic valve of any example herein, particularly any one of examples 39 to 46, wherein portions of the cusp edges are wrapped over the scallop adjustment arms.

Example 53. The prosthetic valve of any example herein, particularly example 52, wherein each scallop adjustment arm comprises a plurality of apertures.

Example 54. The prosthetic valve of any example herein, particularly example 53, wherein the apertures are axially spaced apart from each other.

Example 55. The prosthetic valve of any example herein, particularly any one of examples 53 to 54, wherein the tabs are sutured to the scallop adjustment arms via sutures through the portions of the cusp edges and the apertures.

Example 56. The prosthetic valve of any example herein, particularly example 55, wherein the sutures extend through portions of the cusp edge disposed over opposite sides of the scallop adjustment arms.

Example 57. The prosthetic valve of any example herein, particularly any one of examples 39 to 56, wherein each engagement portion comprises a non-cylindrical socket.

Example 58. The prosthetic valve of any example herein, particularly any one of examples 39 to 56, wherein each engagement portion comprises a proximally oriented extension formed as a drive-screw coupler.

Example 59. The prosthetic valve of any example herein, particularly example 58, wherein each engagement portion comprises an angled surface.

Example 60. The prosthetic valve of any example herein, particularly any one of examples 39 to 59, wherein both adjustable arms of a single adjustable commissural support are laterally spaced from each other and define a gap therebetween.

Example 61. The prosthetic valve of any example herein, particularly example 60, wherein two tabs of adjacent leaflets of the leaflet assembly are attached to the adjustable commissure support, such that each tab is wrapped over one of the adjustable arms.

Example 62. The prosthetic valve of any example herein, particularly example 61, wherein both tabs are wrapped over the respective adjustable arms in opposite directions with respect to each other.

Example 63. The prosthetic valve of any example herein, particularly any one of examples 60 to 62, wherein the gap is at least two times larger than the thickness of the tabs.

Example 64. The prosthetic valve of any example herein, particularly any one of examples 60 to 62, wherein the gap is at least four times larger than the thickness of the tabs.

Example 65. The prosthetic valve of any example herein, particularly any one of examples 60 to 62, wherein the gap is at least six times larger than the thickness of the tabs.

Example 66. The prosthetic valve of any example herein, particularly any one of examples 60 to 62, wherein the gap is at least eight times larger than the thickness of the tabs.

Example 67. A prosthetic valve comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
at least one adjustable commissure support, comprising:
   a clamp coupled to the frame at a first location, the clamp comprising a clamp mid-portion and a couple of opposing side arms continuously extending from the clamp mid-portion; and
   a couple of adjustable arms, each adjustable arm extending proximally from one of the side arms, wherein each adjustable arm comprises an engagement portion;
at least one expansion and locking mechanism, comprising:
   an outer member comprising an outer member coupling recess, wherein the clamp of the adjustable commissure support is clamped over the outer member coupling recess; and
   an inner member, coupled to the frame at a second location spaced apart from the first location, the inner member extending at least partially into the outer member;
wherein movement of the inner member in a first direction, relative to the outer member, causes the frame to foreshorten axially and expand radially;
wherein the side arms are resiliently expandable away from each other, and are inwardly biased toward each other in the absence of an expanding force applied thereto;
wherein the tabs are attached to the adjustable arms;
wherein the adjustable arms are configured to rotate or twist when rotational force is applied to the engagement portions; and
wherein rotation or twisting of the adjustable arms causes the tabs attached thereto, to wind around the adjustable arms.

Example 68. The prosthetic valve of any example herein, particularly example 67, wherein the clamp mid-portion comprises an opening, and wherein the outer member comprises an outer member fastening extension extending radially outward through the opening, and coupled to the frame at the first location.

Example 69. The prosthetic valve of any example herein, particularly example 67, wherein the clamp mid-portion comprises a commissure support fastening extension, extending radially outward from the clamp mid-portion, and coupled to the frame at the first location.

Example 70. The prosthetic valve of any example herein, particularly any one of examples 67 to 69, wherein the outer member coupling recess has a depth which is equal to, or greater than, the thickness of the clamp.

Example 71. The prosthetic valve of any example herein, particularly any one of examples 67 to 70, wherein the outer member coupling recess has a height which is not greater than 110% of the height of the clamp.

Example 72. The prosthetic valve of any example herein, particularly any one of examples 67 to 71, wherein the tabs are wrapped over the adjustable arms.

Example 73. The prosthetic valve of any example herein, particularly any one of examples 67 to 72, wherein each adjustable arm has a non-circular cross section.

Example 74. The prosthetic valve of any example herein, particularly any one of examples 67 to 73, wherein each adjustable arm comprises a plurality of apertures.

Example 75. The prosthetic valve of any example herein, particularly example 74, wherein the apertures are axially spaced apart from each other.

Example 76. The prosthetic valve of any example herein, particularly any one of examples 74 to 75, wherein the tabs are sutured to the adjustable arms via sutures through the tabs and the apertures.

Example 77. The prosthetic valve of any example herein, particularly example 76, wherein the sutures extend through portions of the tab disposed over opposite sides of the adjustment arms.

Example 78. The prosthetic valve of any example herein, particularly any one of examples 67 to 77, wherein the engagement portion comprises a non-cylindrical socket.

Example 79. The prosthetic valve of any example herein, particularly any one of examples 67 to 77, wherein the engagement portion comprises a proximally oriented extension formed as a drive-screw coupler.

Example 80. The prosthetic valve of any example herein, particularly example 79, wherein the engagement portion comprises an angled surface.

Example 81. The prosthetic valve of any example herein, particularly any one of examples 67 to 80, wherein the adjustable arms are rigidly attached to the side arms.

Example 82. The prosthetic valve of any example herein, particularly any one of examples 67 to 80, wherein the adjustable arms are integrally formed with the side arms.

Example 83. The prosthetic valve of any example herein, particularly any one of examples 81 to 82, wherein the adjustable arms are configured to twist between the side arms and the engagement portions, upon application of rotational force to the engagement portions.

Example 84. The prosthetic valve of any example herein, particularly example 83, wherein the adjustable arms comprise a plastically deformable material.

Example 85. The prosthetic valve of any example herein, particularly any one of examples 67 to 70, wherein the adjustable arms are rotationally coupled to the side arms.

Example 86. The prosthetic valve of any example herein, particularly example 85, wherein the adjustable arms are threadedly coupled to the side arms.

Example 87. The prosthetic valve of any example herein, particularly example 86, wherein each side arm comprises a base threaded portion, and wherein each adjustable arm comprises an adjustable arm threaded portion, configured to threadedly engage with the base threaded portion.

Example 88. The prosthetic valve of any example herein, particularly any one of examples 67 to 87, wherein both adjustable arms of a single adjustable commissural support are laterally spaced from each other and define a gap therebetween.

Example 89. The prosthetic valve of any example herein, particularly example 88, wherein two tabs of adjacent leaflets of the leaflet assembly are attached to the adjustable commissure support, such that each tab is wrapped over one of the adjustable arms.

Example 90. The prosthetic valve of any example herein, particularly example 89, wherein both tabs are wrapped over the respective adjustable arms in opposite directions with respect to each other.

Example 91. The prosthetic valve of any example herein, particularly any one of examples 88 to 90, wherein the gap is at least two times larger than the thickness of the tabs.

Example 92. The prosthetic valve of any example herein, particularly any one of examples 88 to 90, wherein the gap is at least four times larger than the thickness of the tabs.

Example 93. The prosthetic valve of any example herein, particularly any one of examples 88 to 90, wherein the gap is at least six times larger than the thickness of the tabs.

Example 94. The prosthetic valve of any example herein, particularly any one of examples 88 to 90, wherein the gap is at least eight times larger than the thickness of the tabs.

Example 95. The prosthetic valve of any example herein, particularly any one of examples 88 to 90, wherein the gap, at a free state of the side arms, is smaller than a width of the outer member at the region of the outer member coupling recess.

Example 96. The prosthetic valve of any example herein, particularly any one of examples 67 to 95, wherein the adjustable arms are offset radially inward with respect to the outer member, defining an offsetting gap therebetween.

Example 97. The prosthetic valve of any example herein, particularly example 96, wherein the offsetting gap is larger than the thickness of the tab.

Example 98. The prosthetic valve of any example herein, particularly example 96, wherein the offsetting gap is at least two times larger than the thickness of the tab.

Example 99. The prosthetic valve of any example herein, particularly example 96, wherein the offsetting gap is at least three times larger than the thickness of the tab.

Example 100. The prosthetic valve of any example herein, particularly example 96, wherein the offsetting gap is at least four times larger than the thickness of the tab.

Example 101. A delivery assembly, comprising:
a prosthetic valve comprising:
   a frame movable between a radially compressed configuration and a radially expanded configuration;
   a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
   a plurality of adjustable commissure supports, each comprising: a support base attached to the frame, and at least one adjustable arm extending proximally from the support base, wherein each adjustable arm comprises an engagement portion;
a delivery apparatus comprising:
   a handle;
   a delivery shaft extending distally from the handle; and
   at least one adjustment assembly extending from the handle through the delivery shaft, the adjustment assembly comprising at least one adjustment arm equipped with a driving head, the driving head releasably coupled to the engagement portion;
wherein the tabs are attached to the adjustable arms;
wherein rotational force applied to the adjustment arms is configured to rotate the engagement portions therewith, thereby causing the adjustable arms to rotate or twist; and
wherein rotation or twisting of the adjustable arms causes the tabs attached thereto, to wind around the adjustable arms.

Example 102. The delivery assembly of any example herein, particularly example 101, wherein the adjustment arm is a torque transmitting arm, configured to transmit torque from the handle to the engagement portion.

Example 103. The delivery assembly of any example herein, particularly any one of examples 101 to 102, wherein each adjustment assembly further comprises an adjustment sleeve extending over the adjustment arm.

Example 104. The delivery assembly of any example herein, particularly example 103, wherein the adjustment sleeve and the adjustment arms are movable longitudinally relative to each other.

Example 105. The delivery assembly of any example herein, particularly any one of examples 101 to 104, wherein the tabs are wrapped over the adjustable arms.

Example 106. The delivery assembly of any example herein, particularly any one of examples 101 to 105, wherein each adjustable arm has a non-circular cross section.

Example 107. The delivery assembly of any example herein, particularly any one of examples 101 to 106, wherein each adjustable arm comprises a plurality of apertures.

Example 108. The delivery assembly of any example herein, particularly example 107, wherein the apertures are axially spaced apart from each other.

Example 109. The delivery assembly of any example herein, particularly any one of examples 106 to 107, wherein the tabs are sutured to the adjustable arms via sutures through the tabs and the apertures.

Example 110. The delivery assembly of any example herein, particularly example 109, wherein the sutures extend through portions of the tab disposed over opposite sides of the adjustment arms.

Example 111. The delivery assembly of any example herein, particularly any one of examples 101 to 110, wherein the engagement portion comprises a non-cylindrical socket.

Example 112. The delivery assembly of any example herein, particularly example 111, wherein the driving head comprises a distally oriented non-circular extension, dimensioned to be inserted into the socket.

Example 113. The delivery assembly of any example herein, particularly any one of examples 101 to 110, wherein the engagement portion comprises a proximally oriented extension formed as a drive-screw coupler.

Example 114. The delivery assembly of any example herein, particularly example 113, wherein the engagement portion comprises an angled surface.

Example 115. The delivery assembly of any example herein, particularly any one of examples 113 to 114, wherein the driving head comprises a distally oriented extension formed as a drive-screw coupler, complementary to the drive-screw coupler shape of the engagement portion.

Example 116. The delivery assembly of any example herein, particularly any one of examples 101 to 115, wherein the adjustable arms are rigidly attached to the support base.

Example 117. The delivery assembly of any example herein, particularly any one of examples 101 to 115, wherein the adjustable arms are integrally formed with the support base.

Example 118. The delivery assembly of any example herein, particularly any one of examples 116 to 117, wherein the adjustable arms are configured to twist between the support base and the engagement portions, upon application of rotational force to the engagement portions.

Example 119. The delivery assembly of any example herein, particularly example 118, wherein the adjustable arms comprise a plastically deformable material.

Example 120. The delivery assembly of any example herein, particularly any one of examples 101 to 115, wherein the adjustable arms are rotationally coupled to the support base.

Example 121. The delivery assembly of any example herein, particularly example 120, wherein the adjustable arms are threadedly coupled to the support base.

Example 122. The delivery assembly of any example herein, particularly example 121, wherein the support base comprises at least one base threaded portion, and wherein each adjustable arm comprises an adjustable arm threaded portion, configured to threadedly engage with the base threaded portion.

Example 123. The delivery assembly of any example herein, particularly any one of examples 101 to 122, wherein the support base further comprises at least one offsetting extension, extending radially therefrom, and wherein the at least one adjustable arm extends from the respective offsetting extension.

Example 124. The delivery assembly of any example herein, particularly example 123, wherein the at least one offsetting extension extends radially away from the support base.

Example 125. The delivery assembly of any example herein, particularly example 123, wherein the at least one offsetting extension extends radially inward with respect to the support base.

Example 126. The delivery assembly of any example herein, particularly any one of examples 123 to 125, wherein the offsetting extension is integrally formed with the support base.

Example 127. The delivery assembly of any example herein, particularly any one of examples 101 to 126, wherein each adjustable commissure support comprises a single adjustable arm.

Example 128. The delivery assembly of any example herein, particularly example 123, wherein two tabs of adjacent leaflets of the leaflet assembly are attached to the adjustable commissure support, such that both tabs are wrapped over the single adjustable arm.

Example 129. The delivery assembly of any example herein, particularly example 128, wherein the leaflets are asymmetric leaflets, each asymmetric leaflet having a short tab and an opposite long tab, and wherein the tabs of adjacent leaflets are wrapped over the single adjustable arm such that the short tab of one leaflet is wrapped around the adjustable arm, in direct contact therewith, and the long tab of the adjacent leaflet is wrapped over the short tab.

Example 130. The delivery assembly of any example herein, particularly any one of examples 123 to 129, wherein each adjustment assembly comprises a single adjustment arm.

Example 131. The delivery assembly of any example herein, particularly any one of examples 101 to 126, wherein each adjustable commissure support comprises two adjustable arms that are laterally spaced from each other and define a gap therebetween.

Example 132. The delivery assembly of any example herein, particularly example 131, wherein two tabs of adjacent leaflets of the leaflet assembly are attached to the adjustable commissure support, such that each tab is wrapped over one of the adjustable arms.

Example 133. The delivery assembly of any example herein, particularly example 132, wherein both tabs are wrapped over the respective adjustable arms in opposite directions with respect to each other.

Example 134. The delivery assembly of any example herein, particularly any one of examples 131 to 133, wherein each adjustment assembly comprises two adjustment arms.

Example 135. A delivery assembly, comprising:
a prosthetic valve comprising:
   a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge;
at least one adjustable commissure support, comprising:
   a clamp coupled to the frame at a first location, the clamp comprising a clamp mid-portion and a couple of opposing side arms continuously extending from the clamp mid-portion; and
   a couple of adjustable arms, each adjustable arm extending proximally from one of the side arms, wherein each adjustable arm comprises an engagement portion;
at least one expansion and locking mechanism, comprising:
   an outer member comprising an outer member coupling recess, wherein the clamp of the adjustable commissure support is clamped over the outer member coupling recess; and
   an inner member, coupled to the frame at a second location spaced apart from the first location, the inner member extending at least partially into the outer member;
a delivery apparatus comprising:
   a handle;
   a delivery shaft extending distally from the handle; and
   at least one adjustment assembly extending from the handle through the delivery shaft, the adjustment assembly comprising at least one adjustment arm equipped with a driving head, the driving head releasably coupled to the engagement portion;
wherein movement of the inner member in a first direction, relative to the outer member, causes the frame to foreshorten axially and expand radially;
wherein the side arms are resiliently expandable away from each other, and are inwardly biased toward each other in the absence of an expanding force applied thereto;
wherein the tabs are attached to the adjustable arms;
wherein rotational force applied to the adjustment arms is configured to rotate the engagement portions therewith, thereby causing the adjustable arms to rotate or twist; and
wherein rotation or twisting of the adjustable arms causes the tabs attached thereto, to wind around the adjustable arms.

Example 136. The delivery assembly of any example herein, particularly example 135, wherein the clamp mid-portion comprises an opening, and wherein the outer member comprises an outer member fastening extension extending radially outward through the opening, and coupled to the frame at the first location.

Example 137. The delivery assembly of any example herein, particularly example 135, wherein the clamp mid-portion comprises a commissure support fastening extension, extending radially outward from the clamp mid-portion, and coupled to the frame at the first location.

Example 138. The delivery assembly of any example herein, particularly any one of examples 135 to 137, wherein the outer member coupling recess has a depth which is equal to, or greater than, the thickness of the clamp.

Example 139. The delivery assembly of any example herein, particularly any one of examples 135 to 138, wherein the outer member coupling recess has a height which is not greater than 110% of the height of the clamp.

Example 140. The delivery assembly of any example herein, particularly any one of examples 135 to 139, wherein the tabs are wrapped over the adjustable arms.

Example 141. The delivery assembly of any example herein, particularly any one of examples 135 to 140, wherein each adjustable arm has a non-circular cross section.

Example 142. The delivery assembly of any example herein, particularly any one of examples 135 to 141, wherein each adjustable arm comprises a plurality of apertures.

Example 143. The delivery assembly of any example herein, particularly example 142, wherein the apertures are axially spaced apart from each other.

Example 144. The delivery assembly of any example herein, particularly any one of examples 142 to 143, wherein the tabs are sutured to the adjustable arms via sutures through the tabs and the apertures.

Example 145. The delivery assembly of any example herein, particularly example 144, wherein the sutures extend through portions of the tab disposed over opposite sides of the adjustment arms.

Example 146. The delivery assembly of any example herein, particularly any one of examples 135 to 145, wherein the engagement portion comprises a non-cylindrical socket.

Example 147. The delivery assembly of any example herein, particularly example 146, wherein the driving head comprises a distally oriented non-circular extension, dimensioned to be inserted into the socket.

Example 148. The delivery assembly of any example herein, particularly any one of examples 135 to 145, wherein the engagement portion comprises a proximally oriented extension formed as a drive-screw coupler.

Example 149. The delivery assembly of any example herein, particularly example 148, wherein the engagement portion comprises an angled surface.

Example 150. The delivery assembly of any example herein, particularly any one of examples 148 to 149, wherein the driving head comprises a distally oriented extension formed as a drive-screw coupler, complementary to the drive-screw coupler shape of the engagement portion.

Example 151. The delivery assembly of any example herein, particularly any one of examples 135 to 150, wherein the adjustable arms are rigidly attached to the side arms.

Example 152. The delivery assembly of any example herein, particularly any one of examples 135 to 150, wherein the adjustable arms are integrally formed with the side arms.

Example 153. The delivery assembly of any example herein, particularly any one of examples 151 to 152, wherein the adjustable arms are configured to twist between the side arms and the engagement portions, upon application of rotational force to the engagement portions.

Example 154. The delivery assembly of any example herein, particularly example 153, wherein the adjustable arms comprise a plastically deformable material.

Example 155. The delivery assembly of any example herein, particularly any one of examples 135 to 150, wherein the adjustable arms are rotationally coupled to the side arms.

Example 156. The delivery assembly of any example herein, particularly example 155, wherein the adjustable arms are threadedly coupled to the side arms.

Example 157. The delivery assembly of any example herein, particularly example 156, wherein each side arm comprises a base threaded portion, and wherein each adjustable arm comprises an adjustable arm threaded portion, configured to threadedly engage with the base threaded portion.

Example 158. The delivery assembly of any example herein, particularly any one of examples 135 to 157, wherein both adjustable arms of a single adjustable commissural support are laterally spaced from each other and define a gap therebetween.

Example 159. The delivery assembly of any example herein, particularly example 158, wherein two tabs of adjacent leaflets of the leaflet assembly are attached to the adjustable commissure support, such that each tab is wrapped over one of the adjustable arms.

Example 160. The delivery assembly of any example herein, particularly example 159, wherein both tabs are wrapped over the respective adjustable arms in opposite directions with respect to each other.

Example 161. The delivery assembly of any example herein, particularly any one of examples 158 to 160, wherein the gap is at least two times larger than the thickness of the tabs.

Example 162. The delivery assembly of any example herein, particularly any one of examples 158 to 160, wherein the gap is at least four times larger than the thickness of the tabs.

Example 163. The delivery assembly of any example herein, particularly any one of examples 158 to 160, wherein the gap is at least six times larger than the thickness of the tabs.

Example 164. The delivery assembly of any example herein, particularly any one of examples 158 to 160, wherein the gap is at least eight times larger than the thickness of the tabs.

Example 165. The delivery assembly of any example herein, particularly any one of examples 158 to 160, wherein the gap, at a free state of the side arms, is smaller than a width of the outer member at the region of the outer member coupling recess.

Example 166. The delivery assembly of any example herein, particularly any one of examples 135 to 165, wherein the adjustable arms are offset radially inward with respect to the outer member, defining an offsetting gap therebetween.

Example 167. The delivery assembly of any example herein, particularly example 166, wherein the offsetting gap is larger than the thickness of the tab.

Example 168. The delivery assembly of any example herein, particularly example 166, wherein the offsetting gap is at least two times larger than the thickness of the tab.

Example 169. The delivery assembly of any example herein, particularly example 166, wherein the offsetting gap is at least three times larger than the thickness of the tab.

Example 170. The delivery assembly of any example herein, particularly example 166, wherein the offsetting gap is at least four times larger than the thickness of the tab.

Example 171. The delivery assembly of any example herein, particularly any one of examples 135 to 170, wherein the adjustment arm is a torque transmitting arm, configured to transmit torque from the handle to the engagement portion.

Example 172. The delivery assembly of any example herein, particularly any one of examples 135 to 171, wherein each adjustment assembly further comprises an adjustment sleeve extending over the adjustment arm.

Example 173. The delivery assembly of any example herein, particularly example 172, wherein

Example 174. A commissure adjustment assembly, comprising:
a valve holder comprising an annular holder body, configured to accommodate a prosthetic valve therein; and
an adjustment handle, releasably attachable to the valve holder, the adjustment handle comprising:
   an annular driver gear;
   a plurality of pinion gears rotatable by the driver gear; and
   a plurality of driving rods, wherein each driving rod extends from a corresponding pinion gear, and wherein each driving rod comprises a driving head, configured to engage with an engagement portion of an engagement portion of adjustable arms of the valve.

Example 175. The commissure adjustment assembly of any example herein, particularly example 174, wherein the valve holder comprises at least one recess, and wherein the adjustment handle comprises at least one clamp configured to engage with the at least one recess.

Example 176. The commissure adjustment assembly of any example herein, particularly any one of examples 174 to 175, wherein the adjustment handle further comprises a handle knob, configured to facilitate rotation of the driver gear.

Example 177. The commissure adjustment assembly of any example herein, particularly example 176, wherein the handle knob and the driver gear are integrally formed.

Example 178. The commissure adjustment assembly of any example herein, particularly any one of examples 174 to 177, wherein the driver gear is an internal gear having internal teeth, and wherein the pinion gears are external gears having external teeth, such that the teeth of at least some of the pinion gears are meshed with the teeth of the driver gear.

Example 179. The commissure adjustment assembly of any example herein, particularly any one of examples 174 to 178, wherein the pinion gears comprise primary pinion gears meshed with the driver gear.

Example 180. The commissure adjustment assembly of any example herein, particularly example 179, further comprising the prosthetic valve positioned within the valve holder, wherein the prosthetic valve comprises a plurality of adjustable commissure supports, wherein each adjustable commissure support comprises a single adjustable arm having the engagement portion, and wherein the driving head of each driving rod extending from a primary pinion gear is engaged with a corresponding engagement portion.

Example 181. The commissure adjustment assembly of any example herein, particularly example 179, further comprising the prosthetic valve positioned within the valve holder, wherein the prosthetic valve comprises a plurality of adjustable commissure supports, wherein each adjustable commissure support comprises two adjustable arm having engagement portions, wherein the driving head of each driving rod extending from a primary pinion gear is engaged with an engagement portion of one adjustable arm of an adjustable engagement support, and wherein each two of the plurality of primary pinion gears having their driving rods engaged with two adjustable arms of the same adjustable commissure supports, are not meshed with each other, and are having their driving rods spaced apart from each other by a gap that is equal to the gap between the respective engagement arms they are engaged with.

Example 182. The commissure adjustment assembly of any example herein, particularly example 179, wherein the pinion gears further comprise secondary pinion gears, which are meshed with the primary pinion gears, but not with the secondary pinion gears.

Example 183. The commissure adjustment assembly of any example herein, particularly example 182, further comprising the prosthetic valve positioned within the valve holder, wherein the prosthetic valve comprises a plurality of adjustable commissure supports, wherein each adjustable commissure support comprises two adjustable arm having engagement portions, and wherein the driving head of each driving rod extending from a primary pinion gear is engaged with an engagement portion of one adjustable arm of an adjustable engagement support, and wherein the driving head of each driving rod extending from the secondary pinion gear meshed with the primary pinion gear is engaged with an engagement portion of the other adjustable arm of the same adjustable engagement support.

Example 184. A method of assembling a commissure assembly, comprising:
wrapping a tab of one leaflet over a single adjustable arm extending from a support base of an adjustable commissure support of a prosthetic valve;
wrapping another tab of an adjacent leaflet over the previous tab; and
suturing both tabs to the adjustable arm via a suture extending through both tabs and apertures of the adjustable arm.

Example 185. The method of any example herein, particularly example 184, wherein the step of suturing comprises passing the suture through portions of the tabs disposed over opposite sides of the adjustment arm.

Example 186. The method of any example herein, particularly any one of examples 184 to 185, wherein the step of wrapping a tab of one leaflet over the adjustable arm comprises wrapping a short tab of the one leaflet over the adjustable arm, and wherein the step of wrapping another tab includes wrapping a long tab of the adjacent leaflet over the short tab.

Example 187. A method of assembling a commissure assembly, comprising:
extending two tabs of two adjacent leaflets through a gap of an adjustable commissure support;
wrapping each tab over a respective adjustable arm, wherein both tabs are wrapped in opposite directions with respect to each other; and
suturing each tab to the respective adjustable arm via a suture extending through the tab and apertures of the adjustable arm.

Example 188. The method of any example herein, particularly example 187, wherein the step of suturing comprises passing the suture through portions of the tab disposed over opposite sides of the adjustment arm.

Example 189. A method for adjusting the tension of leaflets of a prosthetic valve, comprising:
placing a prosthetic valve having a plurality of adjustable commissure supports, within a valve holder of a commissure adjustment assembly;
releasably coupling an adjustment handle to the valve holder, such that driving heads of driving rods extending from pinion gears that are positioned within a driver gear, engage with engagement portions of adjustable arms of the adjustable commissure supports; and
rotating the driver gear to facilitate rotation of the pinion gears rotatable thereby, which in turn rotate the engagement portions therewith.

Example 190. The method of any example herein, particularly example 189, wherein the step of placing the prosthetic valve within the valve holder, comprises a step of expanding the prosthetic valve against an annular holder body of the valve holder.

Example 191. The method of any example herein, particularly any one of examples 189 to 190, wherein each adjustable commissure support comprises a single adjustable arm, wherein the pinion gears comprise primary pinion gears meshed with the driver gear, wherein the step of coupling comprises engaging the driving heads of the driving rods extending from the primary pinion gears with the engagement portions of the single adjustable arms of the adjustable commissure supports, and wherein the step of rotating comprises rotating the driver gear to facilitate rotation of the primary pinion gears meshed therewith.

Example 192. The method of any example herein, particularly any one of examples 189 to 190, wherein each adjustable commissure support comprises two adjustable arms, wherein the pinion gears comprise primary pinion gears meshed with the driver gear, and secondary pinion gears meshed with the primary pinion gears but not with the driver gear, wherein the step of coupling comprises engaging the driving head of the driving rod extending from each primary pinion gear with one adjustable arm of an adjustable commissure support, and engaging the driving head of the driving rod extending from the secondary pinion gear meshed the with the primary pinion gear, with the other adjustable arm of the same adjustable commissure support, and wherein the step of rotating comprises rotating the driver gear to facilitate rotation of the primary pinion gears and the secondary pinion gears in opposite rotational directions.

Example 193. A method for adjusting the tension of leaflets of a prosthetic valve, comprising:
expanding a prosthetic valve that includes a plurality of adjustable commissure supports, to a final expanded diameter;
rotating adjustment arms that extend from a handle of a delivery apparatus, and are coupled via driving heads thereof to engagement portions of adjustable arms of the adjustable commissure supports, to facilitate rotation of the engagement portions therewith; and
disengaging the driving heads from the engagement portions.

Example 194. The method of any example herein, particularly example 193, wherein the step of disengaging the driving heads comprises pulling the driving heads proximally away from the engagement portions.

Example 195. The method of any example herein, particularly example 194, wherein the step of disengaging the driving heads comprises pulling adjustment sleeves that are disposed over the adjustment arms in a proximal direction.

Example 196. The method of any example herein, particularly example 194, wherein the step of disengaging the driving heads comprises simultaneously pulling the adjustment arms with adjustment sleeves disposed thereover.

Example 197. The method of any example herein, particularly any one of examples 193 to 196, wherein the step of rotating the adjustment arms comprises rotating each couple of adjustment arms engaged with two adjustable arms that extend from a single support base, in opposite rotational directions.

Example 198. The method of any example herein, particularly any one of examples 193 to 197, further comprising a step of retrieving the delivery apparatus.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. No feature described in the context of an embodiment is to be considered an essential feature of that embodiment, unless explicitly specified as such.

In view of the many possible embodiments to which the principles of the disclosure may be applied, it should be recognized that the illustrated embodiments are only preferred examples and should not be taken as limiting the scope. Rather, the scope is defined by the following claims. We therefore claim all that comes within the scope and spirit of these claims.

1. A prosthetic valve comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
a plurality of adjustable commissure supports, each comprising: a support base attached to the frame, and at least one adjustable arm extending proximally from the support base, wherein each adjustable arm comprises an engagement portion;
wherein the tabs are attached to the adjustable arms;
wherein the adjustable arms are configured to rotate or twist when rotational force is applied to the engagement portions; and
wherein rotation or twisting of the adjustable arms causes the tabs attached thereto, to wind around the adjustable arms.

2. The prosthetic valve of claim 1, wherein the tabs are wrapped over the adjustable arms.

3. The prosthetic valve of claim 1, wherein each adjustable arm comprises a plurality of apertures.

4. The prosthetic valve of claim 1, wherein each adjustable commissure support comprises two adjustable arms that are laterally spaced from each other and define a gap therebetween.

5. The prosthetic valve of claim 4, wherein two tabs of adjacent leaflets of the leaflet assembly are attached to the adjustable commissure support, such that each tab is wrapped over one of the adjustable arms.

6. The prosthetic valve of claim 5, wherein both tabs are wrapped over the respective adjustable arms in opposite directions with respect to each other.

7. A prosthetic valve comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
a plurality of adjustable commissure supports, each comprising:
a support base attached to the frame;
two adjustable arms coupled to the support base, and rotatable relative thereto, wherein each adjustable arm comprises an engagement portion; and
two scallop adjustment arms, wherein each scallop adjustment arm extends continuously from one of the adjustable arms;
wherein the tabs of adjacent leaflets are attached to the adjustable commissure supports, such that each tab is attached to one of the adjustable arms;
wherein portions of the cusp edge of each leaflet are attached to scallop adjustment arms of adjustable commissure supports on both sides of the leaflet;
wherein both adjustable arms and scallop adjustment arms of a single adjustable commissure support are configured to rotate in opposite directions, when opposite rotational forces are applied to their engagement portions; and
wherein rotation of the adjustable arms and the scallop adjustment arms causes the tabs to wind around the adjustable arms they are attached to, and causes the portions of the cusp edges to wind around the scallop adjustment arms they are attached to.

8. The prosthetic valve of claim 7, wherein the scallop adjustment arm comprises a torque transmitting shaft.

9. The prosthetic valve of claim 7, wherein the tabs are wrapped over the adjustable arms.

10. The prosthetic valve of claim 7, wherein each adjustable arm comprises a plurality of apertures.

11. The prosthetic valve of claim 7, wherein portions of the cusp edges are wrapped over the scallop adjustment arms.

12. The prosthetic valve of claim 11, wherein each scallop adjustment arm comprises a plurality of apertures.

13. The prosthetic valve of claim 7, wherein both adjustable arms of a single adjustable commissural support are laterally spaced from each other and define a gap therebetween.

14. The prosthetic valve of claim 13, wherein two tabs of adjacent leaflets of the leaflet assembly are attached to the adjustable commissure support, such that each tab is wrapped over one of the adjustable arms.

15. The prosthetic valve of claim 14, wherein both tabs are wrapped over the respective adjustable arms in opposite directions with respect to each other.

16. A prosthetic valve comprising:
a frame movable between a radially compressed configuration and a radially expanded configuration;
a leaflet assembly mounted within the frame and comprising a plurality of leaflets configured to regulate flow through the prosthetic valve, wherein each leaflet comprises a rounded cusp end portion defining a cusp edge, a free edge opposite to the cusp edge, and a pair of oppositely-directed tabs separating the cusp edge and the free edge; and
at least one adjustable commissure support, comprising:
a clamp coupled to the frame at a first location, the clamp comprising a clamp mid-portion and a couple of opposing side arms continuously extending from the clamp mid-portion; and
a couple of adjustable arms, each adjustable arm extending proximally from one of the side arms, wherein each adjustable arm comprises an engagement portion;
at least one expansion and locking mechanism, comprising:
an outer member comprising an outer member coupling recess, wherein the clamp of the adjustable commissure support is clamped over the outer member coupling recess; and
an inner member, coupled to the frame at a second location spaced apart from the first location, the inner member extending at least partially into the outer member;
wherein movement of the inner member in a first direction, relative to the outer member, causes the frame to foreshorten axially and expand radially;
wherein the side arms are resiliently expandable away from each other, and are inwardly biased toward each other in the absence of an expanding force applied thereto;
wherein the tabs are attached to the adjustable arms;
wherein the adjustable arms are configured to rotate or twist when rotational force is applied to the engagement portions; and
wherein rotation or twisting of the adjustable arms causes the tabs attached thereto, to wind around the adjustable arms.

17. The prosthetic valve of claim 16, wherein the clamp mid-portion comprises an opening, and wherein the outer member comprises an outer member fastening extension extending radially outward through the opening, and coupled to the frame at the first location.

18. The prosthetic valve of claim 16, wherein the clamp mid-portion comprises a commissure support fastening extension, extending radially outward from the clamp mid-portion, and coupled to the frame at the first location.

19. The prosthetic valve of claim 16, wherein the tabs are wrapped over the adjustable arms.

20. The prosthetic valve of claim 16, wherein each adjustable arm comprises a plurality of apertures.
